(19) United States
(12) Patent Application Publication
(10) Pub. No.: US 2026/0263428 A1
(43) Pub. Date: Sep. 10, 2026

(54) INHIBITORS OF SHORT-CHAIN DEHYDROGENASE ACTIVITY FOR REDUCING GLIAL FIBRILLARY ACIDIC PROTEIN LEVELS

(71) Applicants: CASE WESTERN RESERVE UNIVERSITY, Cleveland, OH (US); The United States Government as Represented by The Department Of Veteran Affairs, Washington, DC (US)

(72) Inventors: Sanford Markowitz, Cleveland, OH (US); Andrew Pieper, Washington, DC (US)

(21) Appl. No.: 18/994,641

(22) PCT Filed: Jul. 19, 2023
(86) PCT No.: PCT/US2023/028134

§ 371 Date: Jan. 15, 2025

Related U.S. Application Data
(60) Provisional application No. 63/390,487, filed on Jul. 19, 2022.

(51) Int. Cl. A61K 31/4365; A61K 31/444; A61K 31/4545; A61K 31/496; A61K 31/506; A61K 31/519; A61K 31/5377; A61P 25/28

(52) U.S. Cl. CPC A61K 31/4365; A61K 31/444; A61K 31/4545; A61K 31/496; A61K 31/506; A61K 31/519; A61K 31/5377; A61P 25/28

(57) ABSTRACT

A method of decreasing blood glial fibrillary acidic protein (GFAP) levels in a subject in need thereof includes administering to the subject a therapeutically effective amount of a 15-PGDH inhibitor. Astroglia cells are the major and perhaps most abundant cell types in the brain. In healthy brain, astrocytes help with providing structural and network support for neurons and interface with the brain vasculature, including the blood-brain-barrier. Functionally, astrocytes are involved with providing neurotrophic factors (such as glial derived neurotrophic factor (GDNF)), and cytokine/chemokine release that influences the global and local inflammatory response environments, as well as working closely with neurons involved in the glutamate-glutamine synthesis/recycling pathway.

### RELATED APPLICATION

This application claims priority from U.S. Provisional Application No. 63/390,847, filed Jul. 19, 2022, the subject matter of which is incorporated herein by reference in its entirety.

### GOVERNMENT FUNDING

This invention was made with government support under CA197442 and GM142002 awarded by the National Institutes of Health. The government has certain rights in the invention.

### BACKGROUND

Astroglia cells are the major and perhaps most abundant cell types in the brain. In healthy brain, astrocytes help with providing structural and network support for neurons and interface with the brain vasculature, including the blood-brain-barrier. Functionally, astrocytes are involved with providing neurotrophic factors (such as glial derived neurotrophic factor (GDNF)), and cytokine/chemokine release that influences the global and local inflammatory response environments, as well as working closely with neurons involved in the glutamate-glutamine synthesis/recycling pathway.

It has been well documented that following neuropertrubation, neuroinjury or when the brain is undergoing neurodegenerative conditions, there is a robust activation of astrocytes (astrogliosis or gliosis). Gliosis can occur in two forms: astrocyte hypertrophy (activation of astroglia with larger, thicker and longer processes) and astrocyte proliferation. Importantly, the astroglia-specific intermediate filament protein called glial fibrillary acidic protein (GFAP) is a critical protein essential for both glial hypertrophy as well as for glial proliferation/cell growth and maturation. Gliosis occurring in a controlled manner might be beneficial following CNS perturbation, but overactivation of the gliosis process is known to have negative impacts on brain recovery or to contribute actively to the neurodegenerative process. In addition to traumatic brain injury, astroglial activation or astrogliosis and GFAP induction might also be involved in the neuro-injury or neuro-repair processes, such as spinal cord injury (SCI), frontal temporal dementia (FTD) and other forms of tauopathies or dementia, multiple sclerosis (MS), stroke (ischemic and hemorrhagic), glioblastoma, vanishing white matter disease, and brain hemorrhage (intracerebral hemorrhage, subarachnoid hemorrhage), Parkinson's disease (PD), Alzheimer's disease (AD), chronic traumatic encephalopathy (CTE), epilepsy, Huntington's disease (HD), Alexander disease, amyotrophic lateral sclerosis (ALS), hypoxic ischemia encephalopathy (HIE), neural damage due to drug or alcohol use or abuse (e.g., from amphetamines, ecstasy/MDMA, or ethanol), prion-related disease, peripheral neuropathy, diabetic neuropathy, and chemotherapy-induced neuropathy and neuropathic pain.

For example, in injured brain activated astroglia in conjunction with fibroblast overgrowth can form a “glial scar” that prevents neuron synaptic reconnection and hinders functional recovery. Similarly, hyperactivated astrogliosis alone or in conjunction with microglia and infiltrating microphages and T cells can evoke an overactivated and sustained neuroinflammatory response that can cause neuronal or oligodendrocyte injury, death or damage to the extracellular matrix.

Under neuroinjury or neurodegenerative and neuro-repair conditions, astroglia cells also can be injured or die. Under these conditions, GFAP (50 kDa; a-isoform) is processed by cellular proteases, such as calpain and caspase-3, and -6, forming C- and N-terminal truncated forms of GFAP with apparent molecular weights of about 44 kDa, 42 kDa, 40 kDa and 38 kDa. The 38 kDa GFAP breakdown product (GBDP-38K) appears to be the major form truncated form. It has been shown that GFAP and GBDPs include GBDP38K are released into extracellular space, including the extracellular fluid and cerebrospinal fluid (CSF), eventually reaching the circulation. Indeed, full length GFAP protein, as well as GFAP fragments (GBDPs) might be cytotoxic or neurotoxic in cell culture conditions and/or in vivo. Furthermore, GFAP under specific conditions and with posttranslational modifications can form protein oligomeric aggregates, which can be cytotoxic as well as trigger neurodegeneration. Taken together, these GFAP and GFAP-BDP (GBDP) can be neurotoxic and a contributor of neurodegeneration.

### SUMMARY

Embodiments described herein relate generally to compositions and methods of decreasing and/or reducing glial fibrillary acidic protein (GFAP) levels in a subject in need thereof, and particularly relates to treating cognitive decline, neural injuries, and/or neurodegeneration, and/or neurodegenerative and/or neuropsychiatric conditions, diseases and/or disorders associated with aberrant and/or elevated GFAP levels and/or astrogliosis.

In some embodiments, the GFAP associated neural injuries, neurodegeneration, and/or neurodegenerative and/or neuropsychiatric conditions, diseases and/or disorders can be associated with enhanced or aberrant 15-PGDH activity in a subject in need thereof. It was found that 15-PDGH activity and GFAP levels are increased in certain neural injuries, neurodegeneration, and/or neurodegenerative and/or neuropsychiatric conditions, diseases, and/or disorders and that 15-PGDH activity could be inhibited, such as with a 15-PGDH inhibitor described herein, to decrease GFAP levels and provide or promote neuroprotection in a subject from astrogliosis, astrocytopathy, axonal degeneration, neuronal cell death, and/or glia cell damage, augment neuronal signaling underlying learning and memory, ameliorate memory loss or cognitive decline, stimulate neuronal regeneration, attenuate or decrease blood brain barrier permeability and/or treat neural injuries, neurodegeneration, and/or neurodegenerative and/or neuropsychiatric conditions, diseases, and/or disorders.

In some embodiments, a method of treating cognitive decline, neural injuries, and/or neurodegeneration, and/or neurodegenerative and/or neuropsychiatric conditions, diseases and/or disorders caused by and/or associated with elevated and/or aberrant GFAP levels and/or 15-PGDH activity in a subject in need thereof includes administering to the subject a therapeutically effective amount of a 15-PGDH inhibitor.

In some embodiments, a therapeutically effective amount of a 15-PGDH inhibitor administered to the subject is an amount effective to decrease blood GFAP levels by at least about 5%, at least about 10%, at least about 15%, at least about 20%, at least about 25%, at least about 30% at least about 35%, at least about 40%, at least about 45%, at least about 50%, at least about 55%, at least about 60%, at least about 65%, at least about 70%, at least about 75% or more.

Other embodiments described herein relate to a method of treating cognitive decline, neural injuries, neurodegeneration, and/or neurodegenerative and/or neuropsychiatric conditions, diseases, and/or disorders associated with aberrant and/or elevated GFAP levels in a subject in need thereof. The method includes measuring GFAP levels in blood of a subject and administering to the subject an amount of a 15-PGDH inhibitor effective to decrease GFAP levels in blood of the subject.

In some embodiments, the method includes measuring GFAP levels in blood of subject following administration of the 15-PGDH inhibitor and comparing the measured GFAP levels to a control, wherein a decrease in blood GFAP levels is indicative of efficacy of the 15-PGDH inhibitor in treating neural injuries, neurodegeneration, and/or neurodegenerative and/or neuropsychiatric conditions, diseases, and/or disorders in the subject. The control can be, for example, a level of GFAP in blood of a subject prior to administration of the 15-PGDH inhibitor.

In some embodiments, the cognitive decline, neural injuries, neurodegeneration, and/or neurodegenerative and/or neuropsychiatric conditions, diseases, and/or disorders associated with aberrant or enhanced GFAP levels and/or astrogliosis can include at least one of subarachnoid hemorrhage, schizophrenia, depression, bipolar disorder, normal aging, epilepsy, traumatic brain injury and/or a visual symptom associated therewith, post-traumatic stress disorder, Parkinson's disease, Parkinson Plus syndromes, Lewy Body Dementia, multiple system atrophy, corticobasal neurodegeneration, progressive supranuclear palsy, Alexander disease, Alzheimer's disease, Alzheimer's disease related dementias, Down syndrome, spinocerebellar ataxia, amyotrophic lateral sclerosis, Huntington's disease, stroke, brain radiation therapy, chronic stress, abuse or cellular toxicity of a neuro-active drug, retinal degeneration, spinal cord injury, peripheral nerve injury, idiopathic peripheral neuropathy, cognitive decline and/or general frailty associated with normal aging and/or chemotherapy, chemotherapy induced neuropathy, concussive injury, peripheral nerve crush injury, peripheral neuropathy, diabetic neuropathy, post-traumatic headache, multiple sclerosis, retinal degeneration and dystrophy, Leber congenital amaurosis, retinitis pigmentosa, cone-rod dystrophy, microphthalmia, anophthalmia, myopia, and hyperopia, spinal cord injury, traumatic spinal cord injury, peripheral nerve injury, retinal neuronal death related diseases, retinal trauma, Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy, Hereditary spastic paraparesis, Friedreich ataxia, Non-Friedreich ataxia, Spinocerebellar atrophies, Amyloidoses, Metabolic-related neurodegenerative disorders, Toxin-related neurodegenerative disorders, Multiple sclerosis, Charcot Marie Tooth, Diabetic neuropathy, Metabolic neuropathies, Endocrine neuropathies, Creutzfeldt-Jacob Disease, Primary progressive aphasia, Frontotemporal Lobar Degeneration, Cortical blindness, Shy-Drager Syndrome, Diffuse cerebral cortical atrophy of non-Alzheimer type, Lewy-body dementia, Pick disease, Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, Tourette syndrome, or viral infection.

In other embodiments, the cognitive decline, neural injuries, and/or neurodegeneration, and/or neurodegenerative and/or neuropsychiatric conditions, diseases and/or disorders associated with aberrant and/or elevated GFAP levels and/or astrogliosisis are also associated with an aberrant level (e.g., decrease or increase in the level) of at least one eicosanoid selected from PGE2, 15-keto-PGE2, PGF2a, 6-keto-PGFla, PGD2, PGJ2, TN-E, TXB2, LTB4, 15-HETE, 12-HETE, 8-HETE, 5-HETE, 17-HDA, 12, 13-DiHOME, 9,10-DiHOME, 14,15-DHET, or 11,12-DHET in neurotissue of the subject. The 15-PGDH inhibitor can be administered to the subject to decrease the GFAP levels and modulate the level of the aberrant eicosanoid to a normal or healthy level in the neurotissue. For example, the neurodegeneration and/or neurodegenerative condition, disease, or disorder can be associated with an increase in GFAP level and an increase in the level of at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in neurotissue (e.g., brain tissue) of the subject, and the 15-PGDH inhibitor can be administered to the subject at amount effective to decrease the GFAP levels and the level of the at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in neurotissue of the subject. In another example, the neurodegeneration and/or neurodegenerative condition, disease, or disorder can be associated with an increase in GFAP levels and a decrease in the level of at least one of PGJ2, TNE, 15-HETE, or 9,10-DiHOME in neurotissue (e.g., brain tissue) of the subject, and the 15-PGDH inhibitor can be administered to the subject at amount effective to decrease GFAP levels and increase the level of the at least one of PGJ2, TNE, 15-HETE, or 9,10-DiHOME in neurotissue (e.g., brain tissue) of the subject.

In some embodiments, the neurotissue can include brain tissue of the subject, such as the hippocampus. The 15-PGDH inhibitor can be administered at an amount effective to stimulate hippocampal neurogenesis.

In other embodiments, the 15-PGDH inhibitor can inhibit the enzymatic activity of recombinant 15-PGDH at an IC₅₀ of less than 1 μM, or preferably at an IC₅₀ of less than 250 nM, or more preferably at an IC₅₀ of less than 50 nM, or more preferably at an IC₅₀ of less than 10 nM, or more preferably at an IC₅₀ of less than 5 nM at a recombinant 15-PGDH concentration of about 5 nM to about 10 nM.

In some of the embodiments described herein, the 15-PGDH inhibitor has the following formula (V):

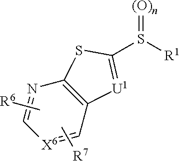

or a pharmaceutically acceptable salt, tautomer, or solvate thereof;
wherein n is 0-2
X⁶ independently is N or CR^{c}
R¹, R⁶, R⁷, and R^{c} are the same or different each independently hydrogen or a substituted or unsubstituted group selected from C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms, C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₅-C₂₀ aryl amino, C₂-C₂₄ alkylamido, C₂-C₂₄ alkylamido substituted with a hydroxyl, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkylethers, phosphates, and phosphate esters, groups incoporating amino acids or other moieties expected to bear positive or negative charge at physiological pH, and combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl; and
U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X═H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X═H, F, Cl, Br, or I), CN, (C═O)—R′, (C═O)N(R′)₂, COOR′ (wherein R′ is H or a lower alkyl group), and R³ or R⁴ may be absent.

### BRIEF DESCRIPTION OF THE DRAWINGS

FIG. 1 shows that 15-PGDH activity is highly enriched in CD11b+ cells (microglial/macrophages) in the brain.

FIG. 2 shows inhibition of 15-PGDH does not suppress pro-inflammatory cytokine production in 5×FAD mice.

FIG. 3 shows that inhibition of 15-PGDH suppresses reactive astrocyte marker, GFAP expression in 5×FAD mice and that such suppression of GFAP expression can be monitored by measuring GFAP levels in blood of 5×FAD mice.

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this application belongs.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.,”, as used herein are non-limiting and are for illustrative purposes only. “Including” and “including but not limited to” are used interchangeably.

The verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

The term “pharmaceutically acceptable salt” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

Additionally, the salts of the compounds described herein, can exist in either hydrated or unhydrated (the anhydrous) form or as solvates with other solvent molecules. Non-limiting examples of hydrates include monohydrates, dihydrates, etc. Nonlimiting examples of solvates include ethanol solvates, acetone solvates, etc.

The term “solvates” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The compounds and salts described herein can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Tautomerizations can he catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

The terms below, as used herein, have the following meanings, unless indicated otherwise:
“Amino” refers to the —NH₂ radical.
“Cyano” refers to the —CN radical.
“Halo” or “halogen” refers to bromo, chloro, fluoro or iodo radical.
“Hydroxy” or “hydroxyl” refers to the —OH radical.
“Imino” refers to the ═NH substituent.
“Nitro” refers to the —NO₂ radical.
“Oxo” refers to the ═O substituent.
“Thioxo” refers to the ═S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl. t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —OR, where Rₐ is an alkyl, alkenyl or alknyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “C_{w}-C_{z} acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₂-C₁₀ alkenyl, or C₂-C₁₀ alkynyl radical as defined above. Unless stated otherwise specifically in the specification, an alkyl carbonyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from phenyl (benzene), aceanthrylene, acenaphthylene, acephenanthrylene, anthracene, azulene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}—R_{c} where R_{b} is an alkylene group as defined above and R_{c} is one or more aryl radicals as defined above. Aralkyl radicals include, but are not limited to, benzyl, diphenylmethyl and the like. Unless stated otherwise specifically in the specification, an aralkyl group can be optionally substituted.

“Aralkenyl” or “arylalkenyl” refers to a radical of the formula —R_{b}—R_{c} where R_{b} is an alkenylene group as defined above and R_{c} is one or more aryl radicals as defined above. Unless stated otherwise specifically in the specification, an aralkenyl group can be optionally substituted.

“Aralkynyl” or “arylalkynyl” refers to a radical of the formula —R_{b}—R_{c} where Rₕ is an alkynylene group as defined above and R_{c} is one or more aryl radicals as defined above. Unless stated otherwise specifically in the specification, an aralkynyl group can be optionally substituted.

“Carbocyclyl,” “carbocyclic ring” or “carbocycle” refers to a ring structure, wherein the atoms which form the ring are each carbon. Carbocyclic rings can comprise from 3 to 20 carbon atoms in the ring. Carbocyclic rings include aryls and cycloalkyl. Cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused, bridged, or spiral ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused, bridged, or spiral ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused, bridged, or spiral ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkynyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused, bridged, and spiral ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, aziridinyl, oextanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, pyridine-one, and the like. The point of attachment of the heterocyclyl, heterocyclic ring, or heterocycle to the rest of the molecule by a single bond is through a ring member atom, which can be carbon or nitrogen. Unless stated otherwise specifically in the specification, a heterocyclyl group can be optionally substituted.

“Heterocyclylalkyl” refers to a radical of the formula —R_{b}—R_{c} where R_{b} is an alkylene group as defined above and Rₑ is a heterocyclyl radical as defined above. Unless stated otherwise specifically in the specification, a heterocyclylalkyl group can be optionally substituted.

“Heterocyclylalkenyl” refers to a radical of the formula —R_{b}—Rₑ where R_{b} is an alkenylene group as defined above and Rₑ is a heterocyclyl radical as defined above. Unless stated otherwise specifically in the specification, a heterocyclylalkenyl group can be optionally substituted.

“Heterocyclylalkynyl” refers to a radical of the formula —R_{b}—Rₑ where R_{b} is an alkynylene group as defined above and Rₑ is a heterocyclyl radical as defined above. Unless stated otherwise specifically in the specification, a heterocyclylalkynyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical one to thirteen carbon atoms and one to six heteroatoms selected from the group consisting of nitrogen, oxygen and sulfur, as the ring member. For purposes of this invention, the heteroaryl radical can be a monocyclic, bicyclic, tricyclic or tetracyclic ring system, which can include fused or bridged ring systems, wherein at least one ring containing a heteroatom ring member is aromatic. The nitrogen, carbon or sulfur atoms in the heteroaryl radical can be optionally oxidized and the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrazolopyridine, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}—R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}—R_{f} where R_{b} is an alkynylene chain as defined above and R_{f} is a heteroaryl radical as defined above. Unless stated otherwise specifically in the specification, a heteroarylalkynyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (e.g., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl, heteroarylalkyl, heteroarylalkenyl, heteroarylalkynyl, etc.) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

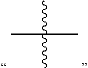

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

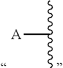

indicates that the chemical entity “A” is bonded to another chemical entity via the point of attachment bond. Furthermore, the specific point of attachment to the non-depicted chemical entity can be specified by inference. For example, the compound

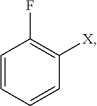

wherein X is

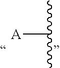

infers that the point of attachment bond is the bond by which X is depicted as being attached to the phenyl ring at the ortho position relative to fluorine.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The term “treating” is art-recognized and includes inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “preventing” is art-recognized and includes stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

A “patient,” “subject,” or “host” to be treated by the subject method may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The terms “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

The term “ED50” is art-recognized. In certain embodiments, ED50 means the dose of a drug, which produces 50% of its maximum response or effect, or alternatively, the dose, which produces a pre-determined response in 50% of test subjects or preparations. The term “LD50” is art-recognized. In certain embodiments, LD50 means the dose of a drug, which is lethal in 50% of test subjects. The term “therapeutic index” is an art-recognized term, which refers to the therapeutic index of a drug, defined as LD50/ED50.

The terms “IC₅₀,” or “half maximal inhibitory concentration” is intended to refer to the concentration of a substance (e.g., a compound or a drug) that is required for 50% inhibition of a biological process, or component of a process, including a protein, subunit, organelle, ribonucleoprotein, etc.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The term “or” as used herein should be understood to mean “and/or”, unless the context clearly indicates otherwise.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

All percentages and ratios used herein, unless otherwise indicated, are by weight.

The terms “healthy” and “normal” are used interchangeably herein to refer to a subject or particular cell or tissue that is devoid (at least to the limit of detection) of a disease condition.

The term “brain injury” includes traumatic injuries and injuries as a result of disease, in particular neurodegenerative diseases and dementias. Thus, “brain injury” includes, but is not limited to mild, moderate, or severe trauma to the brain such as that received in military conflict, sports injury, accidents and falls, and the like, and also includes but is not limited to injury to the brain as a result of any tauopathy or dementia. In a specific embodiment, the brain injury is accompanied by, associated with, or mediated by astrogliosis or astroglial activation.

The term “astrogliosis,” also referred to as “astrocytosis,” “astroglial activation,” or “reactive astrocytosis,” refers to an increase in the number of astrocytes after destruction of neurons due to trauma, infection, ischemia, stroke, immune responses, neurodegenerative disease, or any cause. Astrogliosis also is accompanied by changes in astrocyte morphology and function.

The term “GFAP” refers to intact glial fibrillary acidic protein, an intermediate filament protein encoded by the GFAP gene in humans and expressed in the central nervous system, primarily in astrocytes. All isoforms of the GFAP protein are included in this definition. As used herein, the term also refers to breakdown products of GFAP, including natural and synthetic peptides derived from the sequence of GFAP. Therefore, “GFAP or a fragment thereof” refers to full length GFAP isoforms or any breakdown product, for example, the central core breakdown product GFAP-38K (with residue range about 79-383 in GFAP-α), the N-terminal head region with residue range about 1-72 in GFAP-α, and the C-terminal tail region with residue range about 378-432 in GFAP-α, i.e., the truncated forms of GFAP with apparent molecular weights of about 44 kDa, 42 kDa, 40 kDa and 38 kDa.

“GFAP status” can mean either the level or amount of GFAP at a point in time (such as with a single measure of GFAP), the level or amount of GFAP associated with monitoring (such as with a repeat test on a subject to identify an increase or decrease in GFAP amount), the level or amount of GFAP associated with treatment for traumatic brain injury (whether a primary brain injury and/or a secondary brain injury) or combinations thereof.

“Glasgow Coma Scale” or “GCS” as used herein refers to a 15 point scale for estimating and categorizing the outcomes of brain injury on the basis of overall social capability or dependence on others. The test measures the motor response, verbal response and eye opening response with these values: 1. Motor Response (6—Obeys commands fully; 5—Localizes to noxious stimuli; 4—Withdraws from noxious stimuli; 3—Abnormal flexion, i.e., decorticate posturing; 2—Extensor response, i.e. decerebrate posturing; and 1-—No response); II. Verbal Response (5—Alert and Oriented; 4—Confused, yet coherent, speech; 3—Inappropriate words and jumbled phrases consisting of words; 2—Incomprehensible sounds; and 1-—No sounds); and III. Eye Opening (4—Spontaneous eye opening; 3—Eyes open to speech; 2—Eyes open to pain; and 1-—No eye opening). The final score is determined by adding the values of 1+II+III. The final score can be categorized into four possible levels for survival, with a lower number indicating a more severe injury and a poorer prognosis: Mild (13-15); Moderate Disability (9-12) (Loss of consciousness greater than 30 minutes; Physical or cognitive impairments which may or may resolve; and Benefit from Rehabilitation); Severe Disability (3-8) (Coma: unconscious state. No meaningful response, no voluntary activities); and Vegetative State (Less Than 3) (Sleep wake cycles; Arousal, but no interaction with environment; No localized response to pain).

Moderate brain injury is defined as a brain injury resulting in a loss of consciousness from 20 minutes to 6 hours and a Glasgow Coma Scale of 9 to 12. Severe brain injury is defined as a brain injury resulting in a loss of consciousness of greater than 6 hours and a Glasgow Coma Scale of 3 to 8.

“Glasgow Outcome Scale” as used herein refers to a global scale for functional outcome that rates patient status into one of five categories: Dead, Vegetative State, Severe Disability, Moderate Disability or Good Recovery.

Primary brain injuries occur during the initial insult and result from displacement of the physical structures of the brain. More specifically, a primary brain injury is the physical damage to parenchyma (tissue, vessels) that occurs during the traumatic event, resulting in shearing and compression of the surrounding brain tissue. Secondary brain injuries occur subsequent to the primary injury and may involve an array of cellular processes. More specifically, a secondary brain injury refers to the changes that evolve over a period of time (from hours to days) after the primary brain injury. It includes an entire cascade of cellular, chemical, tissue, or blood vessel changes in the brain that contribute to further destruction of brain tissue.

An injury to the head can be either closed or open (penetrating). A closed head injury refers to a trauma to the scalp, skull or brain where there is no penetration of the skull by a striking object. An open head injury refers a trauma to the scalp, skull or brain where there is penetration of the skull by a striking object. An injury to the head may be caused by physical shaking of a person, by blunt impact by an external mechanical or other force that results in a closed or open head trauma (e.g., vehicle accident, such as with an automobile, plane, train, etc.; blow to the head, such as with a baseball bat, or from a firearm), a cerebral vascular accident (e.g., stroke), one or more falls (e.g., as in sports or other activities), explosions or blasts (collectively, “blast injuries”) and by other types of blunt force trauma. Alternatively, an injury to the head may be caused by the ingestion and/or exposure to a chemical, toxin or a combination of a chemical and toxin. Examples of such chemicals and/or toxins include fires, molds, asbestos, pesticides and insecticides, organic solvents, paints, glues, gases (such as carbon monoxide, hydrogen sulfide, and cyanide), organic metals (such as methyl mercury, tetraethyl lead and organic tin) and/or one or more drugs of abuse. Alternatively, an injury to the head may be caused as a result of a subject suffering from an autoimmune disease, a metabolic disorder, a brain tumor, one or more viruses, meningitis, hydrocephalus, hypoxia or any combinations thereof. In some cases, it is not possible to be certain whether any such event or injury has occurred or taken place. For example, there may be no history on a patient or subject, the subject may be unable to speak, the subject may not be aware of or have full information on what events they were exposed to, etc. Such circumstances are described herein as the subject “may have sustained an injury to the head.” In certain embodiments herein, the closed head injury does not include and specifically excludes a cerebral vascular accident, such as stroke.

“Traumatic Brain Injury” or “TBI” as used interchangeably herein refers to a complex injury with a broad spectrum of symptoms and disabilities. TBI is most often an acute event similar to other injuries. TBI can be classified as “mild,” “moderate,” or “severe.” The causes of TBI are diverse and include, for example, physical shaking by a person, a car accident, injuries from firearms, cerebral vascular accidents (e.g., strokes), falls, explosions or blasts and other types of blunt force trauma. Other causes of TBI include the ingestion and/or exposure to one or more chemicals or toxins (such as fires, molds, asbestos, pesticides and insecticides, organic solvents, paints, glues, gases (such as carbon monoxide, hydrogen sulfide, and cyanide), organic metals (such as methyl mercury, tetraethyl lead and organic tin), one or more drugs of abuse or combinations thereof). Alternatively, TBI can occur in subjects suffering from an autoimmune disease, a metabolic disorder, a brain tumor, hypoxia, one or more viruses, meningitis, hydrocephalus or combinations thereof. Young adults and the elderly are the age groups at highest risk for TBI. In certain embodiments herein, traumatic brain injury or TBI does not include and specifically excludes cerebral vascular accidents such as strokes.

“Mild TBI” as used herein refers to a brain injury where loss of consciousness is brief and usually a few seconds or minutes and/or confusion and disorientation is shorter than 1 hour. Mild TBI is also referred to as a concussion, minor head trauma, minor TBI, minor brain injury, and minor head injury. While Mill and CT scans are often normal, the individual with mild TBI may have cognitive problems, such as headache, difficulty thinking, memory problems, attention deficits, mood swings and frustration.

Mild TBI is the most prevalent TBI and is often missed at time of initial injury. Typically, a subject has a Glasgow Coma scale number of between 13-15 (such as 13-15 or 14-15). Fifteen percent (15%) of people with mild TBI have symptoms that last 3 months or more. Mild TBI is defined as the result of the forceful motion of the head or impact causing a brief change in mental status (confusion, disorientation or loss of memory) or loss of consciousness for less than 30 minutes. Common symptoms of mild TBI include fatigue, headaches, visual disturbances, memory loss, poor attention/concentration, sleep disturbances, dizziness/loss of balance, irritability-emotional disturbances, feelings of depression, and seizures. Other symptoms associated with mild TBI include nausea, loss of smell, sensitivity to light and sounds, mood changes, getting lost or confused, and/or slowness in thinking.

“Moderate TBI” as used herein refers to a brain injury where loss of consciousness and/or confusion and disorientation is between 1 and 24 hours and the subject has a Glasgow Coma scale number of between 9-12. The individual with moderate TBI have abnormal brain imaging results. “Severe TBI” as used herein refers to a brain injury where loss of consciousness is more than 24 hours and memory loss after the injury or penetrating skull injury longer than 24 hours and the subject has a Glasgow Coma scale number between 3-8. The deficits range from impairment of higher level cognitive functions to comatose states. Survivors may have limited function of arms or legs, abnormal speech or language, loss of thinking ability or emotional problems. Individuals with severe injuries can be left in long-term unresponsive states. For many people with severe TBI, long-term rehabilitation is often necessary to maximize function and independence.

Common symptoms of moderate to severe TBI include cognitive deficits including difficulties with attention, concentration, distractibility, memory, speed of processing, confusion, perseveration, impulsiveness, language processing, and/or “executive functions”, not understanding the spoken word (receptive aphasia), difficulty speaking and being understood (expressive aphasia), slurred speech, speaking very fast or very slow, problems reading, problems writing, difficulties with interpretation of touch, temperature, movement, limb position and fine, discrimination, the integration or patterning of sensory impressions into psychologically meaningful data, partial or total loss of vision, weakness of eye muscles and double vision (diplopia), blurred vision, problems judging distance, involuntary eye movements (nystagmus), intolerance of light (photophobia), hearing, such as decrease or loss of hearing, ringing in the ears (tinnitus), increased sensitivity to sounds, loss or diminished sense of smell (anosmia), loss or diminished sense of taste, the convulsions associated with epilepsy that can be several types and can involve disruption in consciousness, sensory perception, or motor movements, control of bowel and bladder, sleep disorders, loss of stamina, appetite changes, regulation of body temperature, menstrual difficulties, dependent behaviors, emotional ability, lack of motivation, irritability, aggression, depression, disinhibition, or denial/lack of awareness.

The terms “neurodegenerative disease”, “neurodegenerative disorder”, or “neurodegenerative condition” are used interchangeably herein to refer to a varied assortment of central nervous system diseases, disorders, and conditions characterised by gradual and progressive loss of neural tissue and/or neural tissue function. A neurodegenerative disease is a class of neurological disorder or disease, and where the neurological disease is characterized by a gradual and progressive loss of neural tissue, and/or altered neurological function, typically reduced neurological function as a result of a gradual and progressive loss of neural tissue.

The term “vascular dementia” is also referred to as “multi-infarct dementia” in the art refers to a group of syndromes caused by different mechanisms all resulting in vascular lesions in the brain. The main subtypes of vascular dementia are, for example vascular mild cognitive impairment, multi-infarct dementia, vascular dementia due to a strategic single infarct (affecting the thalamus, the anterior cerebral artery, the parietal lobes or the cingulate gyrus), vascular dementia due to hemorrhagic lesions, small vessel disease (including, e.g., vascular dementia due to lacunar lesions and Binswanger disease), and mixed Alzheimer's Disease with vascular dementia.

The term “disease”, “disorder”, or “condition” is used interchangeably herein, and refers to any alteration in state of the body or of some of the organs, interrupting or disturbing the performance of the functions and/or causing symptoms such as discomfort, dysfunction, distress, or even death to the person afflicted or those in contact with a person. A disease or disorder can also relate to a distemper, ailing, ailment, malady, disorder, sickness, illness, complaint, inderdisposion or affectation.

The terms “blood-brain barrier” or “BBB” are used interchangeably herein, and are used to refer to the permeability barrier that exists in blood vessels as they travel through the brain tissue that severely restricts and closely regulates what is exchanged between the blood and the brain tissue. The blood brain barrier components include the endothelial cells that form the innermost lining of all blood vessels, the tight junctions between adjacent endothelial cells that are the structural correlate of the BBB, the basement membrane of endothelial cells and the expanded foot processes of nearby astrocytes which cover nearly all of the exposed outer surface of the blood vessel. The BBB prevents most substances in the blood from entering brain tissue, including most large molecules such as Ig, antibodies, complement, albumin and drugs and small molecules.

The term “abnormal BBB” is used to refer to a dysfunctional BBB, for example, where the BBB does not allow transit of molecules that normally transit a functional BBB, for example nutrients and sugars such as glucose. An abnormal BBB can also refer to when the BBB is permeable to molecules that a normally functioning BBB would typically exclude, which is typically referred to “BBB permeability” herein.

The terms “BBB permeability” or “permeable BBB” are commonly referred to by persons in the art as “leaky BBB”. The terms are used interchangeably herein to refer to impaired BBB integrity and increased vascular permeability. For example, a permeable BBB allows transit of molecules through the BBB that an intact BBB would normally exclude from the brain tissue, for example, Ig molecules, complement proteins, serum albumin and numerous other proteins. An assay to determine the presence of a permeable BBB can be, for example, to assess the presence of extravascular Ig in the brain tissue which is normally be restricted to the lumen of blood vessels when the BBB is functioning normally (i.e., when the BBB is not permeable).

Embodiments described herein relate generally to compositions and methods of decreasing and/or reducing glial fibrillary acidic protein (GFAP) levels in a subject in need thereof, and particularly relates to treating cognitive decline, neural injuries, neurodegeneration, and/or neurodegenerative and/or neuropsychiatric conditions, diseases and/or disorders associated with aberrant and/or elevated GFAP levels and/or astrogliosis.

Glial fibrillary acidic protein (GFAP) is a structural protein unique to astrocytes. GFAP is a component in the cytoskeletal structure of astroglial cells and operates in maintaining their mechanical strength, as well as supporting neighboring neurons and the blood-brain barrier (BBB). Because GFAP is enriched in astroglial cells in the CNS, it can be used as a biomarker for diagnosis or prognosis of brain injury or neurodegeneartion. Shortly following brain injury, there is a release of high concentration of GFAP (intact protein, 50 kDa) and its fragments (peptides, also known as breakdown products (BDPs), 38 kDa-44 kDa) from astrocytes into the extracellular fluid and cerebrospinal fluid and blood.

Therefore, GFAP is a pathological hallmark of astrogliosis in brain injury pathology. An increase in GFAP is believed to be an indicator of the astroglial activation and hypertrophy observed following brain injury. Activated astrocytes are known to mediate the neuroinflammation process, including the release for proinflammatory cytokines (e.g., IL-6, TNF-α). Activated astroglia cells also form the so-called glial scar that can further inhibit neuroregeneration. After brain injury and rupture of the blood brain barrier (BBB), GFAP is released from damaged astrocytes, enters the bloodstream where it can trigger an immune response in a subset of brain injury patients.

A brain injury can cause chronic effects, including CNS and systemic sequelae such as cognitive impairment (memory and executive dysfunction), neurological symptoms (headache, sleep disturbance, and pain), neuro-endocrine dysfunction, and mental health impairment (depression, anxiety, apathy, and suicidality).

In some embodiments, the cognitive decline, neural injuries, neurodegeneration, and/or neurodegenerative and/or neuropsychiatric conditions, diseases and/or disorders associated with aberrant and/or elevated GFAP levels and/or astrogliosis can also be associated with enhanced or aberrant 15-PGDH activity in a subject in need thereof. It was found that 15-PDGH activity and GFAP levels are increased in certain neural injuries, neurodegeneration, and/or neurodegenerative and/or neuropsychiatric conditions, diseases, and/or disorders and that 15-PGDH activity could be inhibited, such as with a 15-PGDH inhibitor described herein, to decrease GFAP levels and provide or promote neuroprotection in a subject from astrogliosis, astrocytopathy, axonal degeneration, neuronal cell death, and/or glia cell damage, augment neuronal signaling underlying learning and memory, ameliorate memory loss or cognitive decline, stimulate neuronal regeneration, attenuate or decrease blood brain barrier permeability and/or treat the cognitive decline, neural injuries, neurodegeneration, and/or neurodegenerative and/or neuropsychiatric conditions, diseases and/or disorders associated with aberrant and/or elevated GFAP levels and/or astrogliosis.

In some embodiments, a method of treating cognitive decline, neural injuries, neurodegeneration, and/or neurodegenerative and/or neuropsychiatric conditions, diseases and/or disorders associated with aberrant and/or elevated GFAP levels and/or astrogliosis and/or 15-PGDH activity in a subject in need thereof includes administering to the subject a therapeutically effective amount of a 15-PGDH inhibitor.

In some embodiments, a therapeutically effective amount of a 15-PGDH inhibitor administered to the subject is an amount effective to decrease blood GFAP levels by at least about 5%, at least about 10%, at least about 15%, at least about 20%, at least about 25%, at least about 30% at least about 35%, at least about 40%, at least about 45%, at least about 50%, at least about 55%, at least about 60%, at least about 65%, at least about 70%, at least about 75% or more.

Other embodiments described herein relate to a method of treating cognitive decline, neural injuries, neurodegeneration, and/or neurodegenerative and/or neuropsychiatric conditions, diseases, and/or disorders associated with aberrant and/or elevated GFAP levels in a subject in need thereof. The method includes measuring GFAP levels in blood of a subject and administering to the subject an amount of a 15-PGDH inhibitor effective to decrease GFAP levels in blood of the subject. Methods of measuring GFAP levels in blood of a subject are described, for example, in Abdelhak et al., Blood GFAP as an emerging biomarker in brain and spinal corder disorders; Nature Reviews, Neurology, March 2022, Vol. 19, pgs. 158-172. For example, GFAP levels can be measured in the blood of a subject with a Simoa GFAP Discovery Ket (Quanterix, USA) according to manufacturer's instructions.

In some embodiments, the method includes measuring GFAP levels in blood of subject following administration of the 15-PGDH inhibitor and comparing the measured GFAP levels to a control, wherein a decrease in blood GFAP levels is indicative of efficacy of the 15-PGDH inhibitor in treating cognitive decline, neural injuries, neurodegeneration, and/or neurodegenerative and/or neuropsychiatric conditions, diseases and/or disorders associated with aberrant and/or elevated GFAP levels and/or astrogliosis in a subject in need thereof. The control can be, for example, control is a level of GFAP in blood of a subject prior to administration of the 15-PGDH inhibitor.

In some embodiments, the cognitive decline, neural injuries, neurodegeneration, and/or neurodegenerative and/or neuropsychiatric conditions, diseases and/or disorders are associated with an increase in GFAP levels in neurotissue, such as brain tissue, of at least about 5%, about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 250%, about 300%, about 350%, about 400%, about 450%, about 500%, about 600%, about 700%, about 800%, about 900%, or about 1000% relative to normal or healthy neurotissue. In certain embodiments, administration of a 15-PGDH inhibitor can be used to decrease GFAP in neurotissue of the subject from about 5% to about 200%, about 5% to about 180%, about 5% to about 160%, about 5% to about 140%, about 5% to about 120%, about 5% to about 100%, about 5% to about 80%, about 5% to about 60%, about 5% to about 40%, about 10% to about 200%, about 10% to about 180%, about 10% to about 160%, about 10% to about 140%, about 10% to about 120%, about 10% to about 100%, about 10% to about 80%, about 10% to about 60%, about 30% to about 200%, about 30% to about 180%, about 30% to about 160%, about 30% to about 140%, about 30% to about 120%, about 30% to about 100%, about 30% to about 80%, about 40% to about 200%, about 40% to about 180%, about 40% to about 160%, about 40% to about 140%, about 40% to about 120%, about 40% to about 100%, about 50% to about 200%, about 50% to about 180%, about 50% to about 160%, about 50% to about 140%, about 50% to about 120%, about 60% to about 200%, about 60% to about 180%, about 60% to about 160%, about 60% to about 140%, about 70% to about 200%, about 70% to about 180%, about 70% to about 160%, about 80% to about 200%, about 80% to about 180%, or about 80% to about 200%.

In some embodiments, the cognitive decline, neural injuries, neurodegeneration, and/or neurodegenerative and/or neuropsychiatric conditions, diseases and/or disorders associated with aberrant and/or elevated GFAP levels and/or astrogliosis includes brain injuries. The brain injuries can include any disease or condition involving damage to the brain in which astroglial activation, astrogliosis, or both are involved in the pathologic processes or as biomarkers of the condition. Astrogliosis is a pathologic abnormal increase in the number of astrocytes after destruction of nearby neurons due to trauma, infection, ischemia, autoimmune responses, or neurodegenerative disease such as Alzheimer's disease. Astroglial activation (reactive astrocytes) is a related phenomenon where the astrocytes in the area of an injury undergo changes in molecular expression and morphology as a response to physical or metabolic insult such as infection, ischemia, immune responses, inflammation, hemorrhage, trauma and the like. These cells can protect neurons by taking up toxins from the area and repairing the blood brain barrier, but also can have negative effects that prevent axon regeneration and produce scar tissue.

Brain injuries that can be treated according to the invention include any brain injury that is mediated by astrogliosis/astroglial activation, or that is accompanies by astrogliosis/astroglial activation. These injuries include but are not limited to traumatic brain injury (TBI), stroke (ischemic and hemorrhagic), spinal cord injury (SCI), brain hemorrhage (for example intracerebral hemorrhage and subarachnoid hemorrhage), chronic traumatic encephalopathy (CTE), Alzheimer's disease (AD), frontotemperoral dementia (FTD), Parkinson's disease (PD), multiple sclerosis (MS), and amyloid lateral sclerosis (ALS).

TBI occurs due to physical trauma to the brain, including closed head injury and penetrating head injury. Typically, TBI occurs due to a fall, vehicle collision, work injury, sports injury, violence, and the like. TBI can result in various physical, cognitive and behavioral symptoms, depending on the area of the brain affected and its severity, symptoms which may be permanent. It is a major cause of death and disability. Current treatment focusses on minimizing the damage caused, and prevention.

CTE, also referred to as traumatic encephalopathy syndrome or dementia pugilisitca, is a neurodegenerative condition caused by repeated head injuries, and tends to get worse over time, resulting in dementia. The cause frequently is repeated injury in contact sports, the military, domestic violence, or repeated banging of the head. Firm diagnosis often is made only at autopsy, and no treatment is available and focusses on maintenance and support only.

Neurodegenerative diseases are those which involve progressive loss of neurons or their function, including death of neurons, and which result in a progressive loss of brain function. Neurodegenerative diseases associated with a tauopathy (a pathological aggregation of Tau protein in neurofibrillary or gliofibrillary tangles in the brain) include Alzheimer's disease, FTD, and the like. CTE also sometimes also is classified as this type of neurodegenerative disease.

In some embodiments, the cognitive decline, neural injuries, neurodegeneration, and/or neurodegenerative and/or neuropsychiatric conditions, diseases, and/or disorders associated with aberrant or enhanced GFAP levels and/or astrogliosis can include at least one of subarachnoid hemorrhage, schizophrenia, depression, bipolar disorder, normal aging, epilepsy, traumatic brain injury and/or a visual symptom associated therewith, post-traumatic stress disorder, Parkinson's disease, Parkinson Plus syndromes, Lewy Body Dementia, multiple system atrophy, corticobasal neurodegeneration, progressive supranuclear palsy, Alexander's disease, Alzheimer's disease, Alzheimer's disease related dementias, Down syndrome, spinocerebellar ataxia, amyotrophic lateral sclerosis, Huntington's disease, stroke, brain radiation therapy, chronic stress, abuse or cellular toxicity of a neuro-active drug, retinal degeneration, spinal cord injury, peripheral nerve injury, idiopathic peripheral neuropathy, cognitive decline and/or general frailty associated with normal aging and/or chemotherapy, chemotherapy induced neuropathy, concussive injury, peripheral nerve crush injury, peripheral neuropathy, diabetic neuropathy, post-traumatic headache, multiple sclerosis, retinal degeneration and dystrophy, Leber congenital amaurosis, retinitis pigmentosa, cone-rod dystrophy, microphthalmia, anophthalmia, myopia, and hyperopia, spinal cord injury, traumatic spinal cord injury, peripheral nerve injury, retinal neuronal death related diseases, retinal trauma, Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy, Hereditary spastic paraparesis, Friedreich ataxia, Non-Friedreich ataxia, Spinocerebellar atrophies, Amyloidoses, Metabolic-related neurodegenerative disorders, Toxin-related neurodegenerative disorders, Multiple sclerosis, Charcot Marie Tooth, Diabetic neuropathy, Metabolic neuropathies, Endocrine neuropathies, Creutzfeldt-Jacob Disease, Primary progressive aphasia, Frontotemporal Lobar Degeneration, Cortical blindness, Shy-Drager Syndrome, Diffuse cerebral cortical atrophy of non-Alzheimer type, Lewy-body dementia, Pick disease, Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, Tourette syndrome, or viral infection.

In some embodiments, the cognitive decline, neural injuries, neurodegeneration, and/or neurodegenerative and/or neuropsychiatric conditions, diseases and/or disorders associated with aberrant and/or elevated GFAP levels and/or astrogliosis is also associated with an aberrant level (e.g., decrease or increase in the level) of at least one eicosanoid selected from PGE2, 15-keto-PGE2, PGF2α, 6-keto-PGF1α, PGD2, PGJ2, TN-E, TXB2, LTB4, 15-HETE, 12-HETE, 8-HETE, 5-HETE, 17-HDA, 12, 13-DiHOME, 9,10-DiHOME, 14,15-DHET, or 11,12-DHET in neurotissue of the subject. For example, the neurodegenerative condition, disease, or disorder can be associated with an increase in GFAP levels and an increase or decrease in the level of at least one eicosanoid selected from PGE2, 15-keto-PGE2, PGF2α, 6-keto-PGF1α, PGD2, PGJ2, TN-E, TXB2, LTB4, 15-HETE, 12-HETE, 8-HETE, 5-HETE, 17-HDA, 12, 13-DiHOME, 9,10-DiHOME, 14,15-DHET, or 11,12-DHET of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more in neurotissue of the subject relative to a normal or healthy subject.

In some embodiments, the 15-PGDH inhibitor can be administered to the subject to decrease GFAP levels and modulate the level of the aberrant eicosanoid to a normal or healthy level in the neurotissue. For example, the neurodegeneration and/or neurodegenerative condition, disease, or disorder can be associated with an increase in GFAP levels and an increase in the level of at least one of 15-keto-PGE2, TN-E. TXB2, LTB4, 15-HETE, 12-HETE, 8-HETE, 5-HETE, 17-HDA, 12, 13-DiHOME, 9,10-DiHOME, or 14,15-DHET in neurotissue (e.g., brain tissue) of the subject, and the 15-PGDH inhibitor can be administered to the subject at amount effective to decrease the GFAP levels and the levels of the at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in neurotissue of the subject. In another example, the neurodegeneration and/or neurodegenerative condition, disease, or disorder can be associated with an increase in GFAP levels and a decrease in the levels of at least one of PGJ2, TNE, 15-HETE, or 9,10-DiHOME in neurotissue (e.g., brain tissue) of the subject, and the 15-PGDH inhibitor can be administered to the subject at amount effective to decrease the GFAP levels and increase the levels of the at least one of PGJ2, TNE, 15-HETE, or 9,10-DiHOME in neurotissue (e.g., brain tissue) of the subject.

In some embodiments, the neurotissue can include brain tissue of the subject, such as the hippocampus of the subject. The 15-PGDH inhibitor can be administered at an amount effective to stimulate hippocampal neurogenesis.

Other embodiments described herein relate to a method of treating and/or inhibiting memory loss and/or cognitive decline associated with aberrant or enhanced GFAP levels and/or astrogliosis in a subject in need thereof by administering to the subject a therapeutically effective amount of a 15-PGDH inhibitor. In some embodiments, the memory loss and/or cognitive decline associated with aberrant or enhanced GFAP levels and/or astrogliosis in a subject in need thereof can be caused by and/or associated with neural injuries, neurodegeneration, neurodegenerative and/or neuropsychiatric conditions, diseases, and/or disorders. The therapeutically effective amount of the administered 15-PGDH can be an amount effective to lower GFAP levels and ameliorate memory loss and/or cognitive decline and/or improve memory and/or cognition.

In some embodiments, neurodegenerative condition, disease, or disorder can include at least one of Alzheimer's disease, Lewy body dementia, Vascular dementia, Age-related dementia, Frontotemporal dementia, mixed dementia, or traumatic brain injury.

In still other embodiments, the memory loss and/or cognitive decline associated with aberrant or enhanced GFAP levels and/or astrogliosis is also associated with an aberrant level (e.g., decrease or increase in the level) of at least one eicosanoid selected from PGE2, 15-keto-PGE2, PGF2α, 6-keto-PGF1α, PGD2, PGJ2, TN-E, TXB2, LTB4, 15-HETE, 12-HETE, 8-HETE, 5-HETE, 17-HDA, 12, 13-DiHOME, 9,10-DiHOME, 14,15-DHET, or 11,12-DHET in brain tissue (e.g., hippocampus) of the subject. The 15-PGDH inhibitor can be administered to the subject to lower GFAP levels and modulate the level of the aberrant eicosanoid to a normal or healthy level in the brain tissue. For example, the memory loss and/or cognitive decline can be associated with elevated GFAP levels and an increase in the level of at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in brain tissue of the subject, and the 15-PGDH inhibitor can be administered to the subject at amount effective to decrease the level of GFAP and the level of the at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in brain tissue of the subject. In another example, the memory loss and/or cognitive decline can be associated with an increase in GFAP level and a decrease in the level of at least one of PGJ2, TNE, 15-HETE, or 9,10-DiHOME in brain tissue of the subject, and the 15-PGDH inhibitor can be administered to the subject at amount effective to decrease the GFAP level and increase the levels of the at least one of PGJ2, TNE, 15-HETE, or 9,10-DiHOME in e.g., brain tissue of the subject.

Still other embodiments described herein relate to methods of reducing blood brain barrier permeability in a subject with aberrant or enhanced GFAP levels and/or astrogliosis. The method includes administering to the subject a therapeutically effective amount of a 15-PGDH inhibitor.

In some embodiments, the subject with aberrant or enhanced GFAP levels and/or astrogliosis and blood brain permeability has or is at risk of neurodegenerative condition, disorder, or disease. For example, the subject can have or be at risk of mild cognitive impairment, Alzheimer's disease, Lewy body dementia, Vascular dementia, Age-related dementia, Frontotemporal dementia, mixed dementia, Parkinson's disease, Huntington's disease, multiple sclerosis, diabetic retinopathy, prion disorders, or amyotrophic lateral sclerosis.

In some embodiments, the subject has an aberrant or enhanced GFAP levels and/or astrogliosis and an aberrant level (e.g., decrease or increase in the level) of at least one eicosanoid selected from PGE2, 15-keto-PGE2, PGF2α, 6-keto-PGF1α, PGD2, PGJ2, TN-E, TXB2, LTB4, 15-HETE, 12-HETE, 8-HETE, 5-HETE, 17-HDA, 12, 13-DiHOME, 9,10-DiHOME, 14,15-DHET, or 11,12-DHET in brain tissue. The 15-PGDH inhibitor can be administered to the subject to decrease the GFAP level and modulate the level of the aberrant eicosanoid to a normal or healthy level in the brain tissue. For example, the subject can have an increase in GFAP level and an increase in the levels of at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in neurotissue (e.g., brain tissue) of the subject, and the 15-PGDH inhibitor can be administered to the subject at amount effective to decrease the GFAP levela and the level of the at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in the brain tissue. In another example, the subject can have an increase in GFAP level and a decrease in the level of at least one of PGJ2, TNE, 15-HETE, or 9,10-DiHOME in brain tissue, and the 15-PGDH inhibitor can be administered to the subject at amount effective to decrease the GFAP level and increase the levels of the at least one of PGJ2, TNE, 15-HETE, or 9,10-DiHOME in brain tissue of the subject.

In some embodiments, 15-PGDH inhibitors used to decrease GFAP levels and/or astrogliosis can be identified using assays in which putative inhibitor compounds are applied to cells expressing 15-PGDH and then the functional effects on 15-PGDH activity are determined. Samples or assays comprising 15-PGDH that are treated with a potential inhibitor are compared to control samples without the inhibitor to examine the extent of effect. Control samples (untreated with modulators) are assigned a relative 15-PGDH activity value of 100%. Inhibition of 15-PGDH is achieved when the 15-PGDH activity value relative to the control is about 80%, optionally 50% or 25%, 10%, 5% or 1%.

Agents tested as 15-PGDH inhibitors can be any small chemical molecule or compound. Typically, test compounds will be small chemical molecules, natural products, or peptides. The assays are designed to screen large chemical libraries by automating the assay steps and providing compounds from any convenient source to assays, which are typically run in parallel (e.g., in microtiter formats on microtiter plates in robotic assays).

In some embodiments, the 15-PGDH inhibitor can include a compound having the following formula (I):

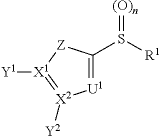

or a pharmaceutically acceptable salt, tautomer, or solvate thereof;
wherein n is 0-2;
Y¹, Y², and R¹ are the same or different and are independently hydrogen or a substituted or unsubstituted group selected from C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms, C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₅-C₂₀ aryl amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkylethers, phosphates, and phosphate esters, groups incorporating amino acids or other moieties expected to bear positive or negative charge at physiological pH, and combinations thereof, and wherein Y¹ and Y² may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl;
U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X═H, F, Cl, Br, or I), CN, (C═O)—R¹, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X═H, F, Cl, Br, or I), CN, (C═O)—R′, (C═O)N(R′)₂, COOR′ (wherein R′ is H or a lower alkyl group), and R³ or R⁴ may be absent;
X¹ and X² are independently N or C, and wherein when X¹ and/or X² are N, Y¹ and/or Y², respectively, are absent; and
Z¹ is O, S, CR^{a}R^{b} or NR^{a}, wherein R^{a} and R^{b} are independently H or a C₁₋₈ alkyl, which is linear, branched, or cyclic, and which is unsubstituted or substituted.

In other embodiments, the 15-PGDH inhibitor can include a compound having the following formula (II):

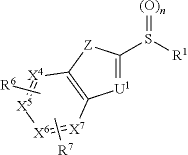

or a pharmaceutically acceptable salt, tautomer, or solvate thereof;
wherein n is 0-2
X⁴, X⁵, X⁶, and X⁷ are independently N or CR^{c};
R¹, R⁶, R⁷, and R^{c} are the same or different and independently hydrogen or a substituted or unsubstituted group selected from C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms, C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₅-C₂₀ aryl amino, C₂-C₂₄ alkylamido, C₂-C₂₄ alkylamido substituted with a hydroxyl, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkylethers, phosphates, and phosphate esters, groups incorporating amino acids or other moieties expected to bear positive or negative charge at physiological pH, and combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl;
U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X═H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X═H, F, Cl, Br, or I), CN, (C═O)—R′, (C═O)N(R′)₂, COOR′ (wherein R′ is H or a lower alkyl group), and R³ or R⁴ may be absent; and
Z¹ is O, S, CR^{a}R^{b} or NR^{a}, wherein R^{a} and R^{b} are independently H or a C₁-s alkyl, which is linear, branched, or cyclic, and which is unsubstituted or substituted.

In yet other embodiments, the 15-PGDH inhibitor can include a compound having the following formula (III) or (IV):

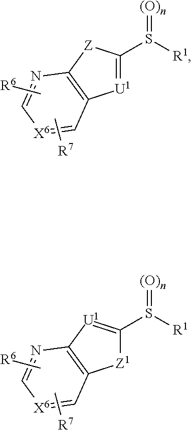

or a pharmaceutically acceptable salt, tautomer, or solvate thereof;
wherein n is 0-2
X⁶ is independently N or CR^{c};
R¹, R⁶, R⁷, and R^{c} are the same or different and independently hydrogen or a substituted or unsubstituted group selected from C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms, C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₂-C₂₄ alkylamido substituted with a hydroxyl, C—C₂₀ aryl amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkylethers, phosphates, and phosphate esters, groups incorporating amino acids or other moieties expected to bear positive or negative charge at physiological pH, and combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl;
U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X═H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X═H, F, Cl, Br, or I), CN, (C═O)—R′, (C═O)N(R′)₂, COOR′ (wherein R′ is H or a lower alkyl group), and R³ or R⁴ may be absent;
Z¹ is O, S, CR^{a}R^{b} or NR^{a}, wherein R^{a} and R^{b} are independently H or a C₁₋₈ alkyl, which is linear, branched, or cyclic, and which is unsubstituted or substituted.

In some embodiments, R¹ is selected from the group consisting of branched, linear, or cyclic alkyl,

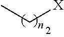

wherein n₂=0-6 and X is any of the following: CF_{y}H_{z} (y+z=3), CCl_{y}H_{z} (y+z=3), OH, OAc, OMe, R⁷¹, OR⁷², CN, N(R⁷³)₂,

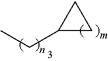

(n₃=0-5, m=1-5), and

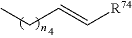

n₄=0-5).

In other embodiments, R⁶ and R⁷ can each independently be one of the following:

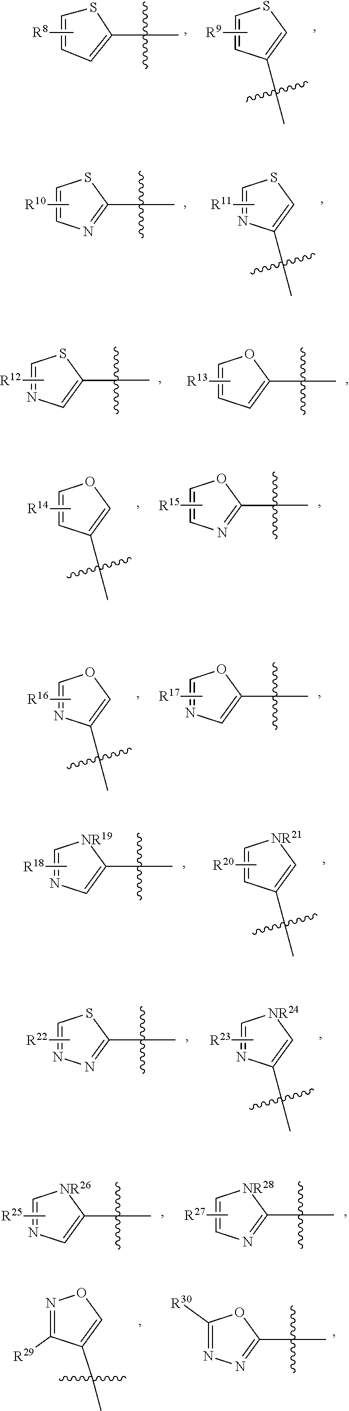

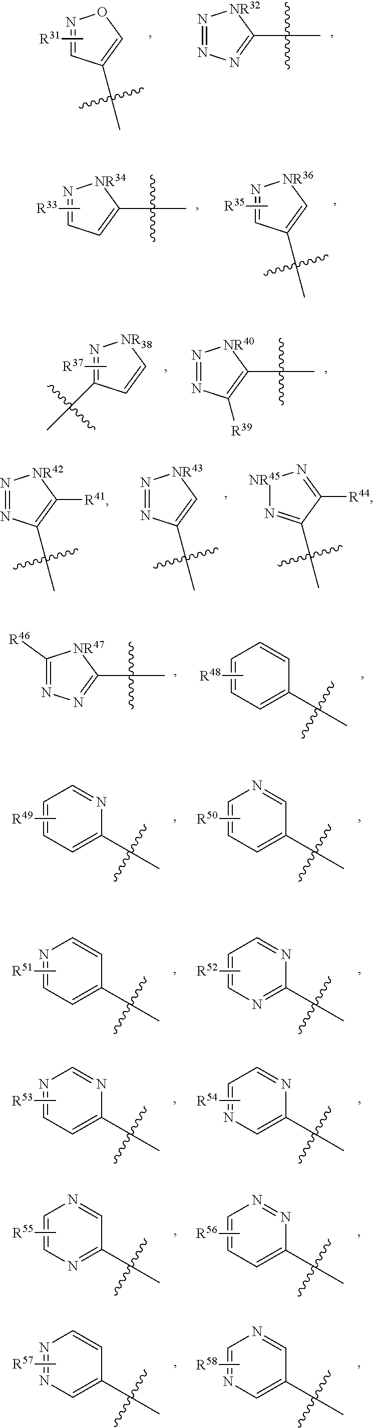

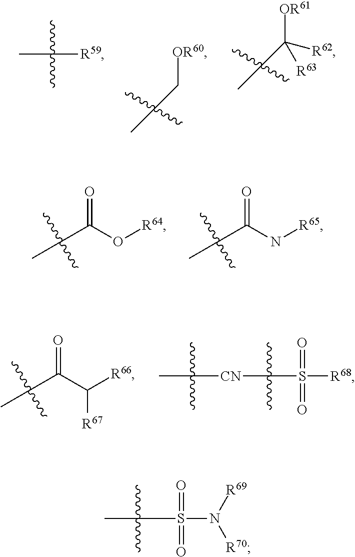

each R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, and R⁷⁴ are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-6 ring atoms, (wherein from 1-3 of the ring atoms is independently selected from N, NH, N(C₁-C₆ alkyl), NC(O)(C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO— alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano (—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₁-C₂₄ alkyl amino substituted with hydroxyl, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C—C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O—)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof, or a pharmaceutically acceptable salt, tautomer, or solvate thereof.

In still other embodiments, R⁶ and R⁷ can independently be a group that improves aqueous solubility, for example, a phosphate ester (—OPO₃H₂), a phenyl ring linked to a phosphate ester (—OPO₃H₂), a phenyl ring substituted with one or more methoxyethoxy groups, or a morpholine, or an aryl or heteroaryl ring substituted with such a group.

In other embodiments, the 15-PGDH inhibitor can include a compound having the following formula (V):

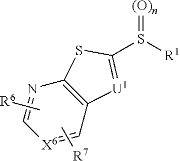

or a pharmaceutically acceptable salt, tautomer, or solvate thereof;
wherein n is 0-2
X⁶ is independently is N or CR^{c}
R¹, R⁶, R⁷, and R^{c} are the same or different each independently hydrogen or a substituted or unsubstituted group selected from C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms, C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₅-C₂₀ aryl amino, C₂-C₂₄ alkylamido, C₂-C₂₄ alkylamido substituted with a hydroxyl, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkylethers, phosphates, and phosphate esters, groups incorporating amino acids or other moieties expected to bear positive or negative charge at physiological pH, and combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl; and
U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X═H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X═H, F, Cl, Br, or I), CN, (C═O)—R′, (C═O)N(R′)₂, COOR′ (wherein R′ is H or a lower alkyl group), and R³ or R⁴ may be absent.

In some embodiments, R¹ is selected from the group consisting of branched, linear, or cyclic alkyl,

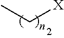

wherein n₂=0-6 and X is any of the following: CF_{y}H_{z} (y+z=3), CCl_{y}H_{z} (y+z=3), OH, OAc, OMe, R⁷¹, OR⁷², CN, N(R⁷³)₂,

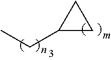

(n₃=0-5, m=1-5), and

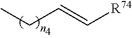

(n₄=0-5).

In other embodiments, R⁶ and R⁷ can each independently be one of the following:

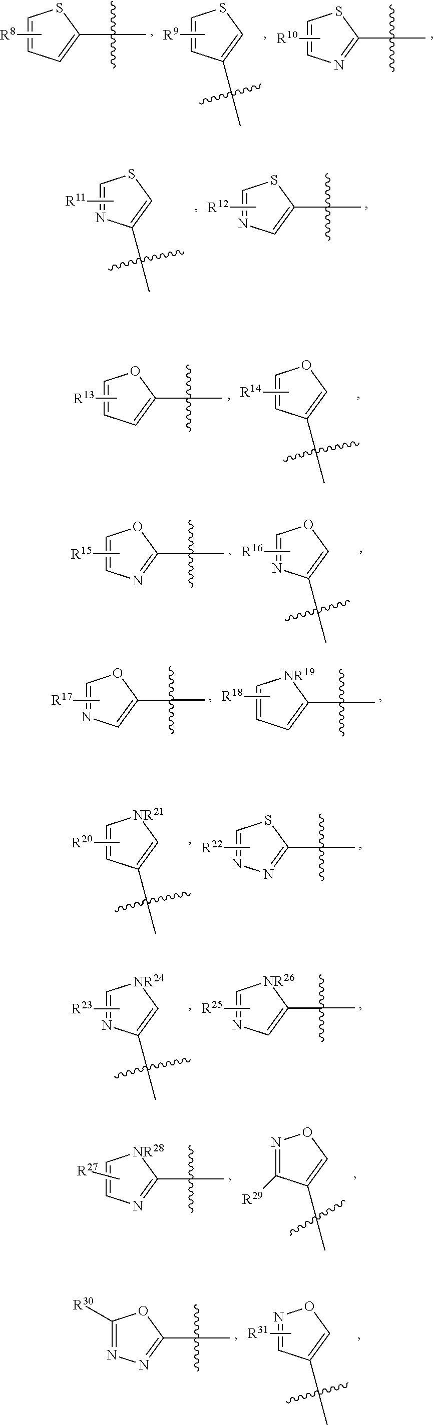

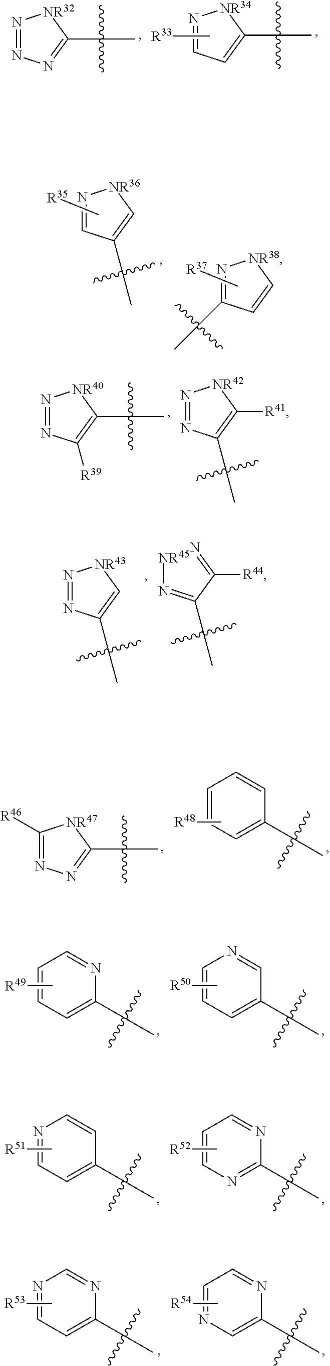

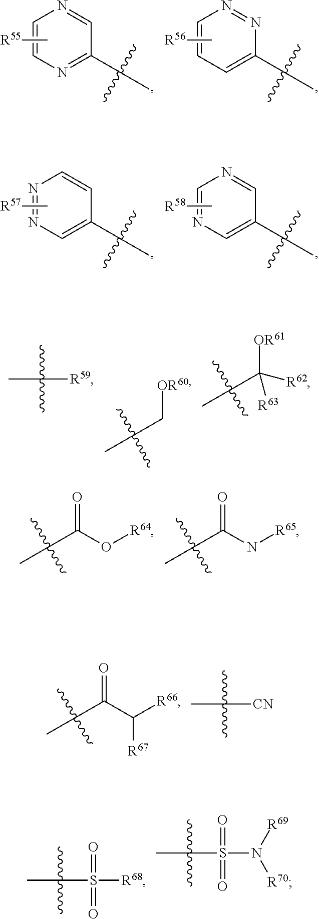

each R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, and R⁷⁴, are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-6 ring atoms, (wherein from 1-3 of the ring atoms is independently selected from N, NH, N(C₁-C₆ alkyl), NC(O)(C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C—C₂₀ aryloxy, acyl (including C₂-C₂₄ alkylcarbonyl (—CO— alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano (—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₁-C₂₄ alkyl amino substituted with hydroxyl, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof, or a pharmaceutically acceptable salt, tautomer, or solvate thereof.

In still other embodiments, R⁶ and R⁷ can independently be a group that improves aqueous solubility, for example, a phosphate ester (—OPO₃H₂), a phenyl ring linked to a phosphate ester (—OPO₃H₂), a phenyl ring substituted with one or more methoxyethoxy groups, or a morpholine, or an aryl or heteroaryl ring substituted with such a group.

In other embodiments, the 15-PGDH inhibitor can include a compound having a structure of formula (IA):

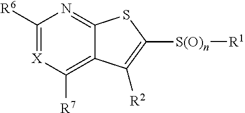

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)alkyl;
R⁶ is heterocyclyl or heteroaryl, each of which is optionally substituted with one or more R³;
R⁷ is alkyl, haloalkyl, cycloalkyl, aryl, heterocyclyl, heteroaryl, —C(O)-alkyl,
—C(O)O-alkyl, or —C(O)NR⁵-alkyl, each of which is optionally substituted with one or more R⁴;
R³ is oxo, —OH, —O-alkylene-OH, —O-alkylene-N(R⁵)₂, —N(R⁵)₂, —N(R′)(alkylene-OH), —N(R⁵)(alkylene-O-alkyl), alkyl, -alkylene-OH, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ alkyl, wherein the cycloalkyl and the heterocyclyl is each optionally substituted with R¹⁰;
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, -alkylene-O-alkyl, alkoxy, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from O, S(O)ₜ, or N;
R¹⁰ is —OH, halogen, alkyl, or alkoxy;
X is N or CH;
m is 0, 1, or 2;
n is 0, 1, or 2; and
t is 0, 1, or 2.

In some embodiment, the 15-PGDH inhibitor can include a compound having a structure of formula (IIA):

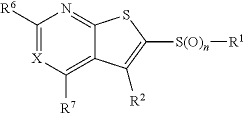

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)alkyl;
R⁶ is heterocyclycl or heteroaryl, each of which is optionally substituted with one or more R³; R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl;
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, alkoxy, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from O, S(O)ₜ, or N;
X is N or CH;
m is 0, 1, or 2;
n is 0, 1, or 2; and
t is 0, 1, or 2.

In some embodiments, R¹ is C₁-C₆ alkyl, C₁-C₆ haloalkyl, 3- to 6-membered cycloalkyl, —(C₁-C₆ alkylene)-(3- to 6-membered cycloalkyl), —(C₁-C₆ alkylene)-(C₁-C₆ alkoxy), 3- to 6-membered heterocyclyl, or —(C₁-C₆ alkylene)-(3- to 6-membered heterocyclyl).

In other embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, or —(CH₂)ₚ-cyclohexyl; wherein p is 1, 2, or 3.

In still other embodiments, R² is —NH₂.

In some embodiments, R⁶ is 5- to 6-membered heterocyclycl or 5- to 10-membered heteroaryl, each of which is optionally substituted with one or more R³.

In other embodiments, R⁶ is 5- to 6-membered heteroaryl optionally substituted with one or more R³.

In still other embodiments, R⁶ is 8- to 10-membered bicyclic heteroaryl optionally substituted with one or more R³.

In some embodiments, R⁷ is C₁-C₆ alkyl, C₁-C₆ haloalkyl, 3- to 6-membered cycloalkyl, 6- to 10-membered aryl, 3- to 6-membered heterocyclyl, 5- to 10-membered heteroaryl, —C(O)(C₁-C₆ alkyl), —C(O)O(C₁-C₆ alkyl), or —C(O)NR⁵(C₁-C₆ alkyl), each of which is optionally substituted with one or more R⁴.

In other embodiments, R⁷ is C₁-C₆ alkyl, C₁-C₆ haloalkyl, 3- to 6-membered cycloalkyl, phenyl, 3- to 6-membered heterocyclyl, or 5- to 10-membered heteroaryl, each of which is optionally substituted with one or more R⁴.

In still other embodiments, R⁷ is C₁-C₆ haloalkyl, 3- to 6-membered cycloalkyl, phenyl, 5- to 10-membered heteroaryl each of which is optionally substituted with one or more R⁴.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In other embodiments, R³ is —(C₁-C₃ alkyl)OH, —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In other embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In still other embodiments, R³ is —NHCH₂CH₂OH or —N(CH₃)CH₂CH₂OH.

In some embodiments, R⁴ is halogen, —CN, —N(R⁵)₂, —OH, —O—(C₁-C₆ alkylene)-OH, —S(O)ₘ(C₁-C₆ alkyl), —C(O)(C₁-C₆ alkyl), —C(O)-(3- to 6-membered cycloalkyl), C₁-C₆ alkyl, C₁-C₆ haloalkyl, 3- to 6-membered cycloalkyl, or 3- to 6-membered heterocyclyl.

In some embodiments, n is 1.

In other embodiments, the compound has the structure of formula (IIIA):

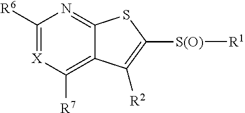

or a pharmaceutically acceptable salt, tautomer, or solvate thereof, wherein:
R¹ is 3- to 6-membered cycloalkyl, —(C₁-C₆ alkylene)-(3- to 6-membered cycloalkyl), 3- to 6-membered heterocyclyl, or —(C₁-C₆ alkylene)-(3- to 6-membered heterocyclyl);
R² is —NH₂, CN, or —NHC(O)alkyl;
R⁶ is heterocyclyl or heteroaryl, each of which is optionally substituted with one or more R³.
R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl,
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from 0, S(O)ₜ, or N;
X is N or CH;
m is 0, 1, or 2; and
t is 0, 1, or 2.

In some embodiments, R¹ is 3- to 5-membered cycloalkyl or —(C₁-C₆ alkylene)-(3- to 5-membered cycloalkyl).

In other embodiments, R¹ is cyclobutyl.

In still other embodiments, R¹ is a bicyclic 4- to 6-membered cycloalkyl.

In some embodiments, R⁷ is C₁-C₆ alkyl, C₁-C₆ haloalkyl, 3- to 6-membered cycloalkyl, 6- to 10-membered aryl, 3- to 6-membered heterocyclyl, 5- to 10-membered heteroaryl, —C(O)(C₁-C₆ alkyl), —C(O)O(C₁-C₆ alkyl), or —C(O)NR⁵(C₁-C₆ alkyl), each of which is optionally substituted with one or more R⁴.

In other embodiments, R⁷ is C₁-C₄ alkyl, C₁-C₆ haloalkyl, 3- to 6-membered cycloalkyl, phenyl, 3- to 6-membered heterocyclyl, or 5- to 10-membered heteroaryl, each of which is optionally substituted with one or more R⁴. In other embodiments, R⁷ is C₁-C₆ haloalkyl, 3- to 6-membered cycloalkyl, phenyl, 3- to 6-membered heterocyclyl, or 5- to 10-membered heteroaryl, each of which is optionally substituted with one or more R⁴.

In other embodiments, R⁷ is C₁-C₃ alkyl, C₁-C₃ haloalkyl, 3-membered cycloalkyl, phenyl, 4-membered heterocyclyl, or 5- to 6-membered heteroaryl, each of which is optionally substituted with one or more R⁴. In other embodiments, R⁷ is C₁-C₃ haloalkyl, 3-membered cycloalkyl, phenyl, 4-membered heterocyclyl, or 5- to 6-membered heteroaryl, each of which is optionally substituted with one or more R⁴.

In other embodiments, R⁷ is —CF₃, isopropyl, cyclopropyl, phenyl, pyridyl, pyrazole, or triazole, each of which is optionally substituted with one or more R⁴. In other embodiments, R⁷ is —CF₃, cyclopropyl, phenyl, pyridyl, pyrazole, or triazole, each of which is optionally substituted with one or more R⁴.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In other embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In still other embodiments, R³ is —NHCH₂CH₂OH or —N(CH₃)CH₂CH₂OH.

In some embodiments and without being limited by theory, Applicants surprisingly and unexpectedly discovered that substituents at the R⁷ position could be modified to improve hERG activity, including hERG inhibition (IC₅₀), blockade, and efflux ratio. For example, in some embodiments, certain 6- to 10-membered aryls (e.g., optionally substituted phenyl) and 5- to 10-membered heteroaryls (e.g., optionally substituted pyridyls, pyrazoles, and triazoles) were observed to have beneficial hERG properties. In some embodiments, certain C₁-C₆ haloalkyls (e.g., —CF₃) exhibited improved hERG inhibition (IC50), while also improving half-life and solubility.

In other embodiments, the compound has the structure of formula (IVA):

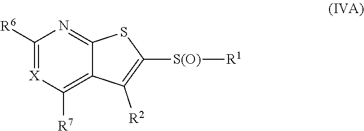

or a pharmaceutically acceptable salt, tautomer, or solvate thereof, wherein:
R¹ is cycloalkyl, alkylene-cycloalkyl, alkylene-alkoxy, heterocyclyl, or alkylene-heterocyclyl;
R² is —NH₂, CN, or —NHC(O)alkyl;
R⁶ is heterocyclyl or heteroaryl, each of which is optionally substituted with one or more R³;
R⁷ is C₁-C₆ haloalkyl, aryl or heteroaryl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, -alkylene-COOH, —C(O)O-alkyl, or —S(O)ₘ-alkyl;
or alternative, two R⁵ together with the N atom to which they are attached can form a 4- to 7-membered heterocycle, optionally containing an additional heteroatom selected from O, S, or N, and wherein the heterocycle is optionally substituted with R⁸;
R⁴ is halogen, alkyl, or alkoxy;
X is N or CH; and
m is 0, 1, or 2.

In some embodiments, R⁷ is —CF₃, pyridyl, pyrazole, phenyl, or triazole, each of which is optionally substituted with R⁴.

In other embodiments, R⁷ is —CF₃, pyridyl, fluorophenyl, or a triazole optionally substituted with halogen or methyl.

In other embodiments, R⁷ is —CF₃,

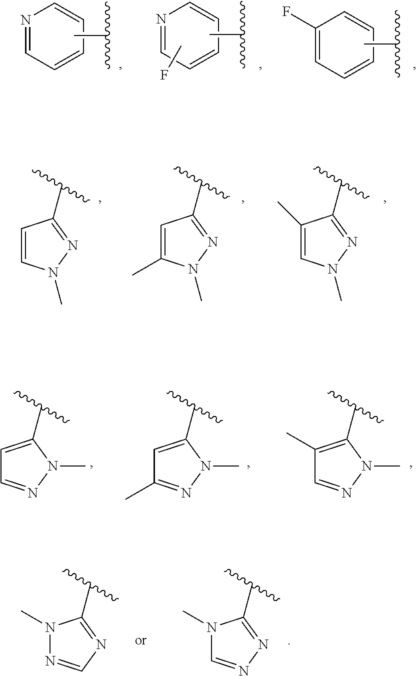

In still other embodiments, R⁷ is —CF₃.

In still other embodiments, R⁷ is

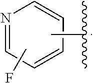

In some embodiments, R⁷ is

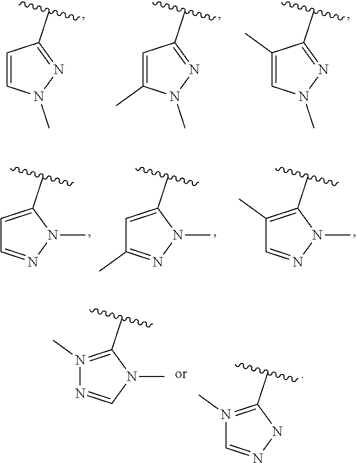

In some embodiments, R⁷ is

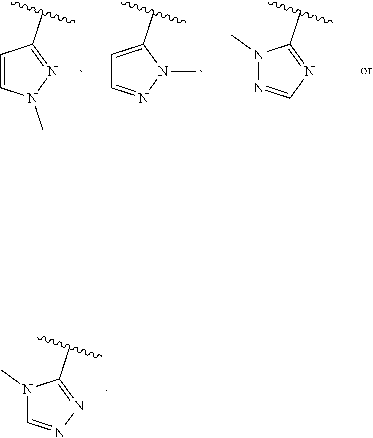

In other embodiments, R⁶ is 8- to 10-membered bicyclic heteroaryl optionally substituted with one or more R³.

In some embodiments and without being limited by theory, Applicants surprisingly and unexpectedly discovered that 3- to 6-membered cycloalkyls at the R⁷ position can improve solubility while maintaining PDGH activity.

In other embodiments, the compound has the structure of formula (VA):

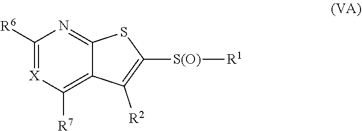

or a pharmaceutically acceptable salt, tautomer, or solvate thereof, wherein:
R¹ is cycloalkyl, -alkylene-cycloalkyl, -alkylene-alkoxy, heterocyclyl, or -alkylene-heterocyclyl;
R² is —NH₂, CN, or —NHC(O)alkyl;
R⁶ is heterocyclyl or heteroaryl, each of which is optionally substituted with one or more R³;
R⁷ is 3- to 6-membered cycloalkyl, optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl;
R⁴ is halogen, —CN, —NH₂, —OH, or C₁-C₃ alkyl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, -alkylene-COOH, —C(O)O-alkyl, or —S(O)ₘ-alkyl;
or alternative, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
X is N or CH;
m is 0, 1, or 2.

In some embodiments, R⁷ is cyclopropyl.

In other embodiments, R¹ is 3- to 6-membered cycloalkyl, —(C₁-C₆ alkylene)-(3- to 6-membered cycloalkyl), —(C₁-C₆ alkylene)-(C₁-C₆ alkoxy), 3- to 6-membered heterocyclyl, or —(C₁-C₆ alkylene)-(3- to 6-membered heterocyclyl).

In some embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, or —(CH₂)ₚ-cyclohexyl; wherein p is 1, 2, or 3.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In some embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In other embodiments, R³ is —NHCH₂CH₂OH or —N(CH₃)CH₂CH₂OH.

In some embodiments and without being limited by theory, Applicants surprisingly and unexpectedly discovered that the R⁶ position can be substituted with certain R³ groups to improve solubility and activity.

In other embodiments, the compound has the structure of formula (VIA):

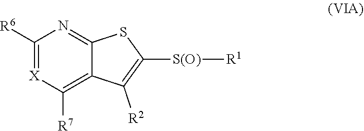

or a pharmaceutically acceptable salt, tautomer, or solvate thereof, wherein:
R¹ is cycloalkyl, -alkylene-cycloalkyl, -alkylene-alkoxy, heterocyclyl, or -alkylene-heterocyclyl;
R² is —NH₂, CN, or —NHC(O)alkyl;
R⁶ is heterocyclyl or heteroaryl, each of which is substituted with one or more R³;
R⁷ is haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl);
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, haloalkyl, cycloalkyl, heterocyclyl, or -alkylene-aryl optionally substituted with R⁸;
each R⁵ is independently H, C₁-C₆ alkyl, —(C₁-C₆ alkylene)-OH optionally substituted with —OH, -alkylene-NH₂, -alkylene-N(R⁹)₂, -alkylene-O-alkylene-OH, -alkylene-O-alkylene-NH₂, —C(O)(C₁-C₆ alkyl), —C(O)O(C₁-C₆ alkyl), alkylene-COOH, or —S(O)ₘ(C₁-C₆ alkyl);
or alternative, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
X is N or CH;
m is 0, 1, or 2.

In some embodiments, R³ is —O—(C₁-C₆ alkylene)-N(R⁵)₂, —N(R⁵)₂ or —N(R⁵)(C₁-C₆ alkylene-OH).

In other embodiments, R⁵ is H, C₁-C₆ alkyl, —(C₁-C₆ alkylene)-OH, or —S(O)₂(C₁-C₃ alkyl).

In some embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In other embodiments, R³ is —NHCH₂CH₂OH or —N(CH₃)CH₂CH₂OH.

In still other embodiments, R³ is —NHCH₂CH₂OH.

In some embodiments, R⁶ is 5- to 6-membered heterocyclyl or 5- to 10-membered heteroaryl, each of which is optionally substituted with one or more R³.

In other embodiments, R⁶ is 5- to 6-membered heteroaryl optionally substituted with one or more R³.

In some embodiments, R⁶ is furan, thiophene, pyrrole, thiazole, isothiazole, oxazole, isooxazole, pyrazole, imidazole, triazole, pyridine, pyrimidine, pyridazine, or pyrazine, each optionally substituted with one or more R³.

In other embodiments, R⁶ is thiazole, imidazole, oxazole, pyridine, or pyrimidine.

In some embodiments, R⁶ is 8- to 10-membered bicyclic heteroaryl optionally substituted with one or more R³.

In other embodiments, R⁶ is 5- to 6-membered heterocyclyl, optionally substituted with one or more R³, selected from morpholine, pyridine-one, or piperidine.

In some embodiments, R⁷ is C₁-C₃ haloalkyl, 3-membered cycloalkyl, phenyl, 4-membered heterocyclyl, or 5- to 6-membered heteroaryl, each of which is optionally substituted with one or more R⁴.

In other embodiments, R⁷ is —CF₃, cyclopropyl, phenyl, pyrazole, pyridyl, or triazole, each of which is optionally substituted with one or more R⁴.

In some embodiments, the compound has the structure of formula (VITA):

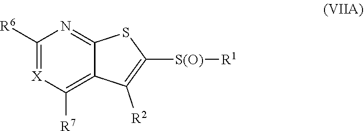

or a pharmaceutically acceptable salt, tautomer, or solvate thereof, wherein:
R¹ is cycloalkyl, alkylene-cycloalkyl, alkylene-alkoxy, heterocyclyl, or alkylene-heterocyclyl;
R² is —NH₂, CN, or —NHC(O)alkyl;
R⁶ is fused bicyclic heterocyclyl or fused bicyclic heteroaryl, each of which is optionally substituted with one or more R³;
R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)-alkyl, —C(O)O-alkyl, or —S(O)ₘ-alkyl; R⁴ is oxo, halogen, —CN, —N(R⁵)₂, —OH, —O-alkylene-OH, —S(O)ₘ-alkyl, —C(O)— alkyl, —C(O)-cycloalkyl, alkyl, haloalkyl, cycloalkyl, heterocyclyl, or -alkylene-aryl optionally substituted with R⁵;
each R⁵ is independently, H, alkyl, -alkylene-OH optionally substituted with —OH, -alkylene-O-alkylene-OH, -alkylene-O-alkylene-NH₂, —C(O)-alkyl, —C(O)O-alkyl, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
X is N or CH; and
m is 0, 1, or 2.

In some embodiments, R⁶ is 8- to 10-membered fused bicyclic heteroaryl, each of which is optionally substituted with one or more R³.

In some embodiments, R⁷ is C₁-C₆ alkyl, C₁-C₆ haloalkyl, 3- to 6-membered cycloalkyl, 6- to 10-membered aryl, 3- to 6-membered heterocyclyl, 5- to 10-membered heteroaryl, —C(O)(C₁-C₆ alkyl), —C(O)O(C₁-C₆ alkyl), or —C(O)NR⁵(C₁-C₆ alkyl), each of which is optionally substituted with one or more R⁴.

In other embodiments, R⁷ is C₁-C₄ alkyl, C₁-C₆ haloalkyl, 3- to 6-membered cycloalkyl, phenyl, 3- to 6-membered heterocyclyl, or 5- to 10-membered heteroaryl, each of which is optionally substituted with one or more R⁴. In other embodiments, R⁷ is C₁-C₆ haloalkyl, 3- to 6-membered cycloalkyl, phenyl, 3- to 6-membered heterocyclyl, or 5- to 10-membered heteroaryl, each of which is optionally substituted with one or more R⁴.

In still other embodiments, R⁷ is C₁-C₃ alkyl, C₁-C₆ haloalkyl, 3- to 6-membered cycloalkyl, phenyl, 3- to 6-membered heterocyclyl, or 5- to 6-membered heteroaryl, each of which is optionally substituted with one or more R⁴.

In other embodiments, R⁷ is C₁-C₃ alkyl, C₁-C₆ haloalkyl, 3- to 6-membered cycloalkyl, phenyl, pyrazole, pyridyl, or triazole, each of which is optionally substituted with one or more R⁴. In other embodiments, R⁷ is C₁-C₆ haloalkyl, 3- to 6-membered cycloalkyl, phenyl, pyrazole, pyridyl, or triazole, each of which is optionally substituted with one or more R⁴.

In other embodiments, R⁷ is —CF₃, isopropyl, cyclopropyl, phenyl, pyridyl, or triazole, each of which is optionally substituted with one or more R⁴. In other embodiments, R⁷ is —CF₃, cyclopropyl, phenyl, pyridyl, or triazole, each of which is optionally substituted with one or more R⁴.

In some embodiments, R¹ is C₁-C₆ alkyl, C₁-C₆ haloalkyl, 3- to 6-membered cycloalkyl, —(C₁-C₆ alkylene)-(3- to 6-membered cycloalkyl), —(C₁-C₆ alkylene)-(C₁-C₆ alkoxy), 3- to 6-membered heterocyclyl, or —(C₁-C₆ alkylene)-(3- to 6-membered heterocyclyl).

In other embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, or —(CH₂)ₚ-cyclohexyl; wherein p is 1, 2, or 3.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In other embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In some embodiments, R³ is —NHCH₂CH₂OH or —N(CH₃)CH₂CH₂OH.

In other embodiments, R⁴ is halogen, —CN, —N(R⁵)₂, —OH, —O—(C₁-C₆ alkylene)-OH, —S(O)ₘ(C₁-C₆ alkyl), —C(O)(C₁-C₆ alkyl), —C(O)-(3- to 6-membered cycloalkyl), C₁-C₆ alkyl, C₁-C₆ haloalkyl, 3- to 6-membered cycloalkyl, or 3- to 6-membered heterocyclyl.

In some embodiments, the compound has the structure of formula (VIIIA):

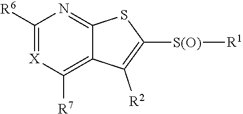

or a pharmaceutically acceptable salt, tautomer, or solvate thereof, wherein:
R¹ is cyclobutyl or —(C₁-C₄ alkylene)-(C₁-C₃ alkoxy);
R² is —NH₂, CN, or —NHC(O)alkyl;
R⁶ is heterocyclyl or heteroaryl, each of which is optionally substituted with one or more R³;
R⁷ is —CF₃, isopropyl,

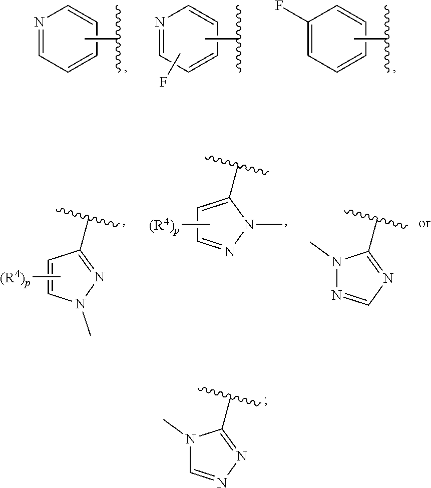

R³ is oxo, —OH, —O-alkylene-OH, —O-alkylene-N(R⁵)₂, —N(R⁵)₂, —N(R⁵)(alkylene-OH), —N(R⁵)(alkylene-O-alkyl), alkyl, -alkylene-OH, haloalkyl, cycloalkyl, heterocyclyl,
   —C(O)N(R⁵)₂, —C(O)N(R⁵)(alkylene-OH), —C(O)-alkyl, —C(O)O-alkyl, or —S(O)ₘ-alkyl, wherein the cycloalkyl and the heterocyclyl is each optionally substituted with R¹⁰
R⁴ is C₁-C₃ alkyl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from O, S(O)ₜ, or N;
R¹⁰ is —OH, halogen, alkyl, or alkoxy;
X is N or CH;
m is 0, 1, or 2;
p is 0 or 1; and
t is 0, 1, or 2.

In still other embodiments, R² is —NH₂.

In some embodiments, R⁶ is 5- to 6-membered heterocyclyl or 5- to 10-membered heteroaryl, each of which is optionally substituted with one or more R³.

In other embodiments, R⁶ is 5- to 6-membered heteroaryl optionally substituted with one or more R³.

In still other embodiments, R⁶ is 8- to 10-membered bicyclic heteroaryl optionally substituted with one or more R³.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In other embodiments, R³ is —(C₁-C₃ alkyl)OH, —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In still other embodiments, R³ is —NHCH₂CH₂OH or —N(CH₃)CH₂CH₂OH.

In some embodiments, R⁴ is halogen, —CN, —N(R⁵)₂, —OH, —O—(C₁-C₆ alkylene)-OH, —S(O)ₘ(C₁-C₆ alkyl), —C(O)(C₁-C₆ alkyl), —C(O)-(3- to 6-membered cycloalkyl), C₁-C₆ alkyl, C₁-C₆ haloalkyl, 3- to 6-membered cycloalkyl, or 3- to 6-membered heterocyclyl.

In some embodiments, n is 1.

In some embodiments, the compound has the structure of formula (IXA):

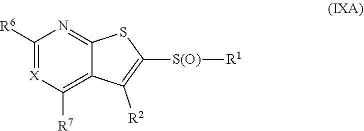

or a pharmaceutically acceptable salt, tautomer, or solvate thereof, wherein:
R¹ is cyclobutyl or —(C₁-C₄ alkylene)-(C₁-C₃ alkoxy);
R² is —NH₂, CN, or —NHC(O)alkyl;
R⁶ is

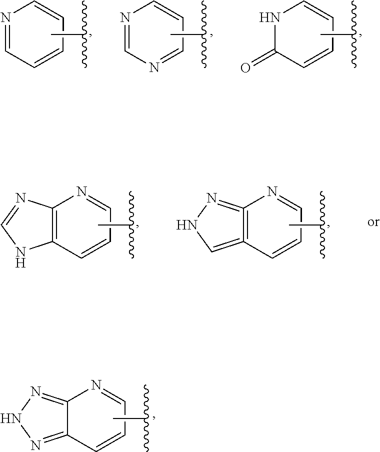

each of which is optionally substituted with one or more R³;
R⁷ is —CF₃, isopropyl, cyclopropyl, cyclobutyl,

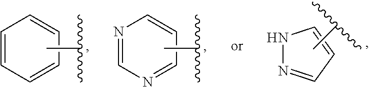

each of which is optionally substituted with one or more R⁴;
R³ is —NH₂, —NH(C₁-C₃ alkyl), —NH(C₁-C₄ alkylene)-OH, or C₁-C₃ alkyl;
R⁴ is C₁-C₃ alkyl; and
X is N or CH.

In some embodiments of Formula (IXA), R¹ is cyclobutyl. In some embodiments of Formula (IXA), R¹—(C₁-C₄ alkylene)-(C₁-C₃ alkoxy). In some embodiments of Formula (IXA), the R¹—(C₁-C₄ alkylene)-(C₁-C₃ alkoxy) is —(C₂-C₃ alkylene)-(C₁ alkoxy).

In some embodiments of Formula (IXA), R² is —NH₂.

In some embodiments of Formula (IXA), R⁶ is

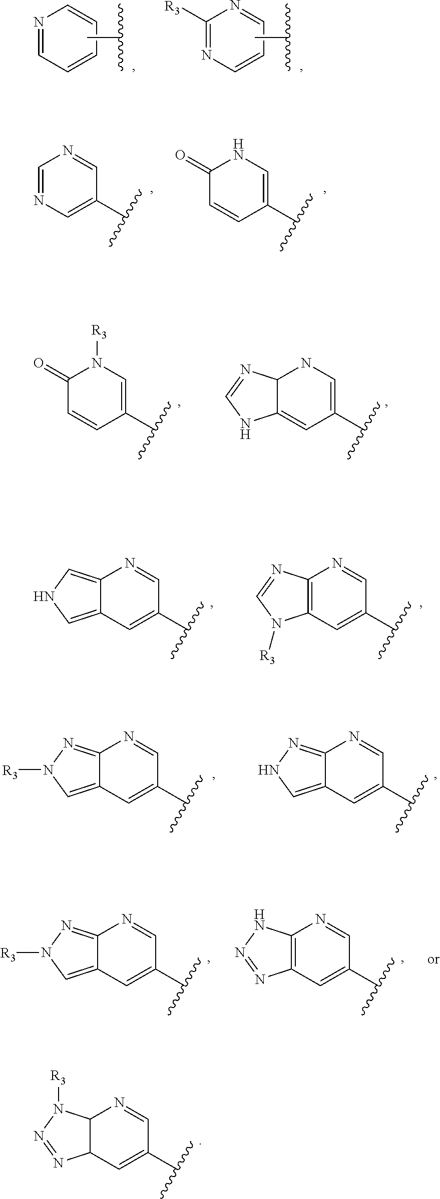

In some embodiments of Formula (IXA), R³ is —NH₂. In some embodiments of Formula (IXA), R³ is —NH(C₁-C₃ alkyl). In some embodiments of Formula (IXA), R³ is —NH(C₁-C₄ alkylene)-OH (e.g., —NH(C₂-C₄ alkylene)-OH). In some embodiments of Formula (IXA), R³ is C₁-C₃ alkyl (e.g., methyl or ethyl).

In some embodiments, of Formula (IXA), R⁷ is —CF₃, isopropyl, cyclopropyl, or cyclobutyl. In some embodiments, of Formula (IXA), R⁷ is isopropyl. In some embodiments of Formula (VII), R⁷ is

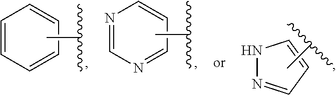

each of which is optionally substituted with one or more R⁴. In some embodiments, each R⁴ is independently selected from methyl or ethyl.

In some embodiments of Formula (IXA), X is —CH.

Examples of compounds having formulas (I), (II), (III), (IV), (V), (IA), (IIA), (IIIA), (IVA), (VA), (VIA), (VIIA), (VIIIA), and (IXA) are described in U.S. Patent Application Publication Nos. 2015/0072998, 2017/0165241, 2017/0173028, 2018/0118756, WO2018/218251, and WO2020/106998, all of which are incorporated by reference in their entirety.

For example, the 15-PGDH inhibitor can include a compound selected from the group consisting of:

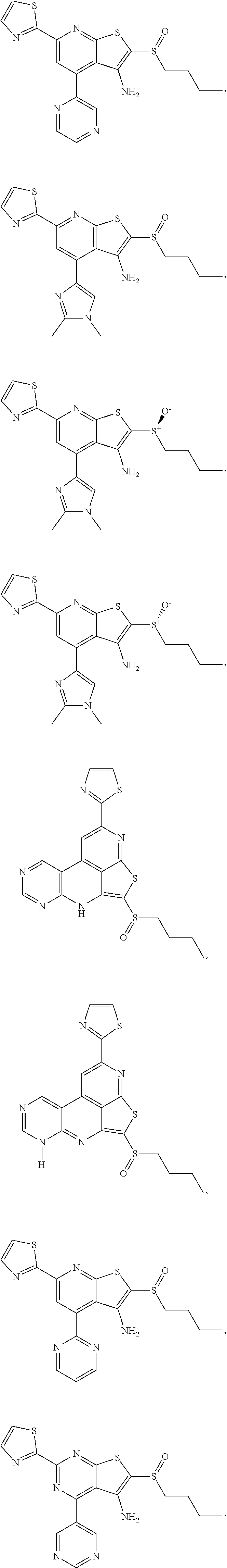

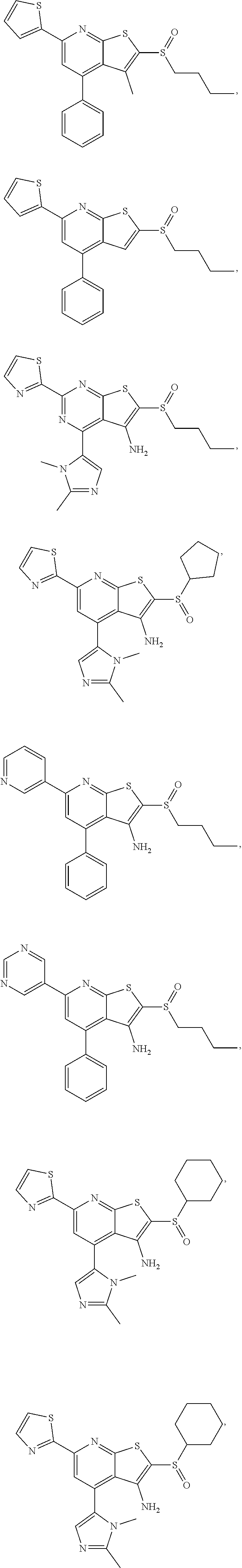

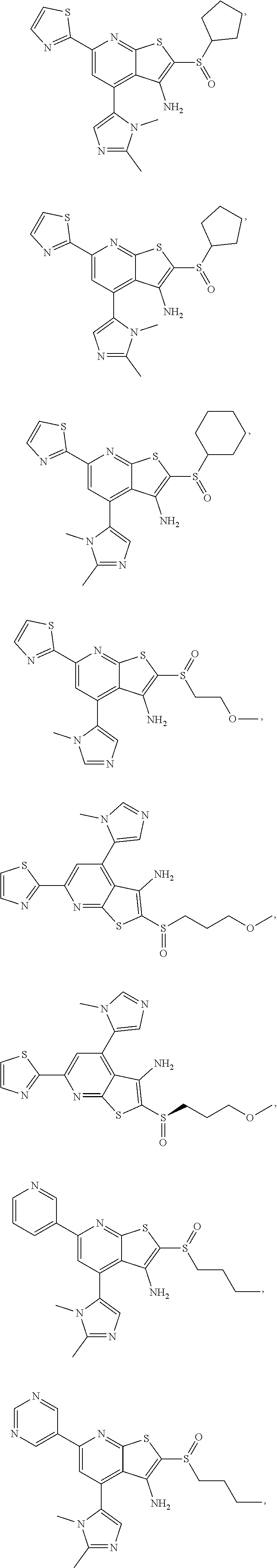

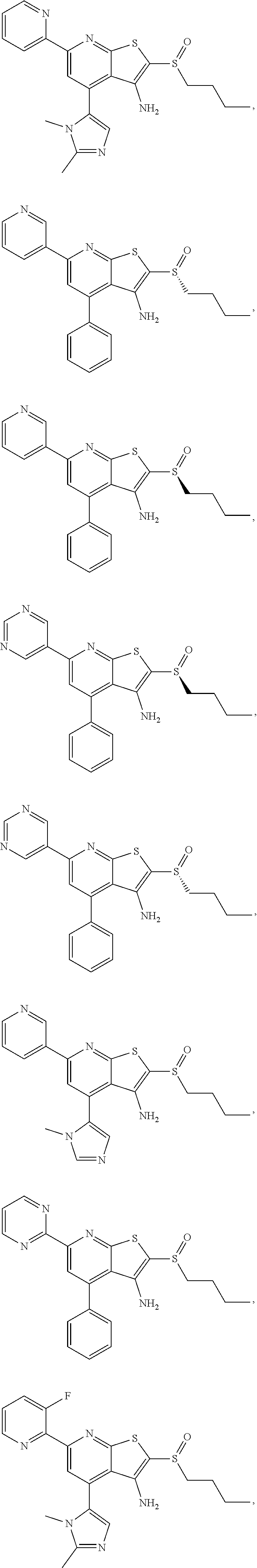

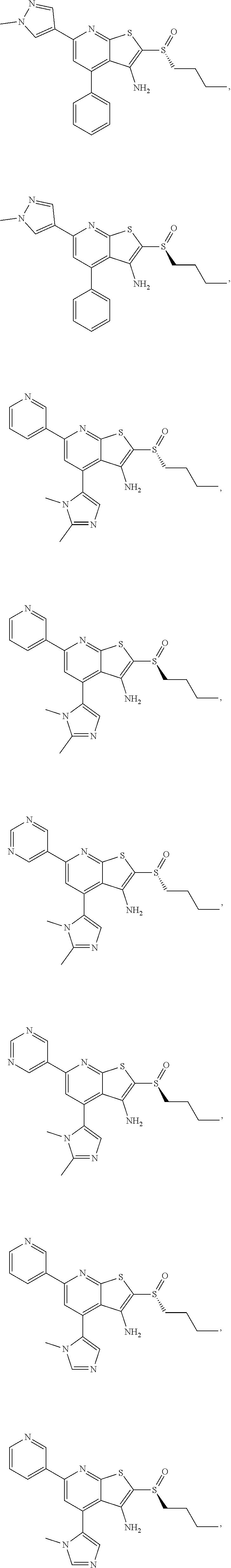

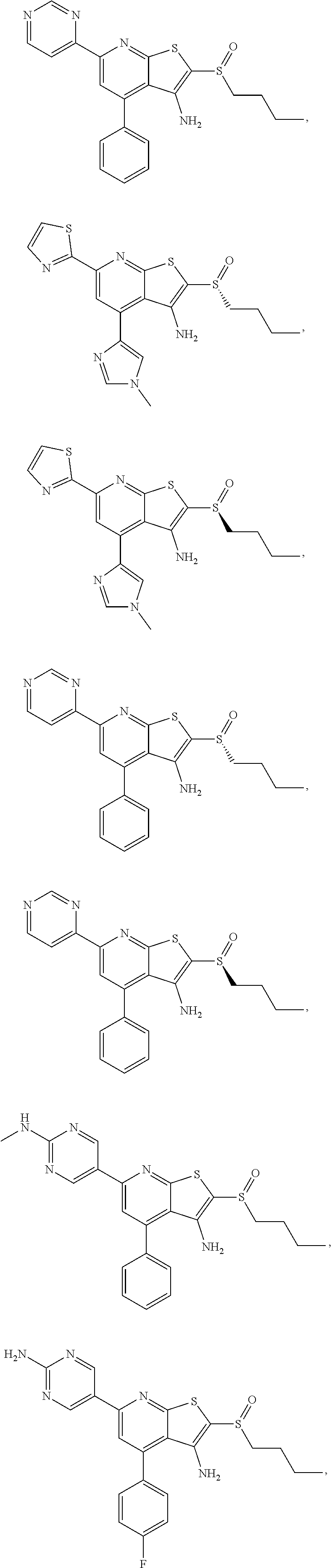

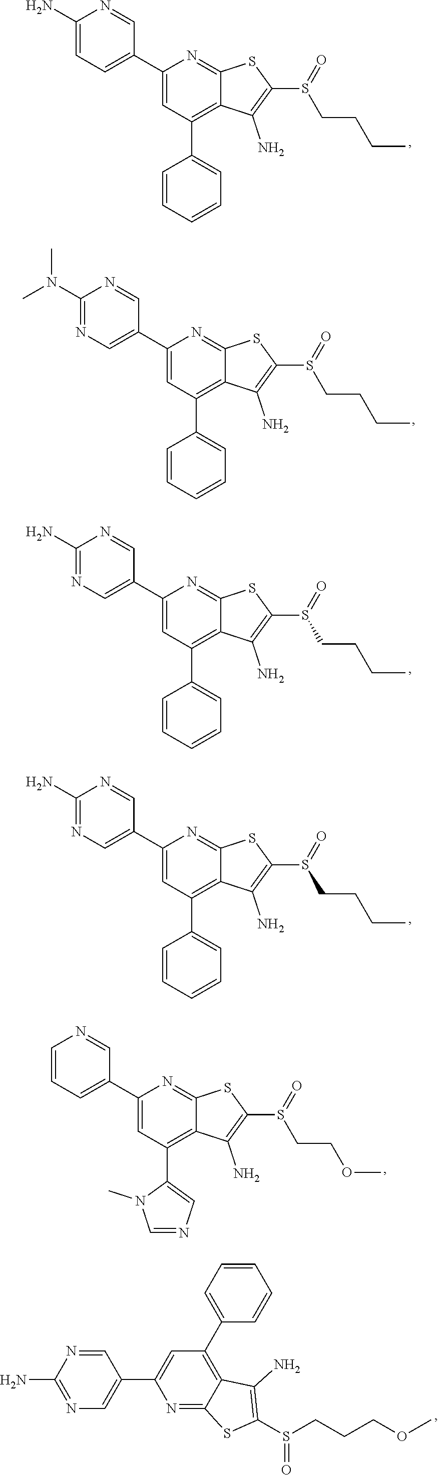

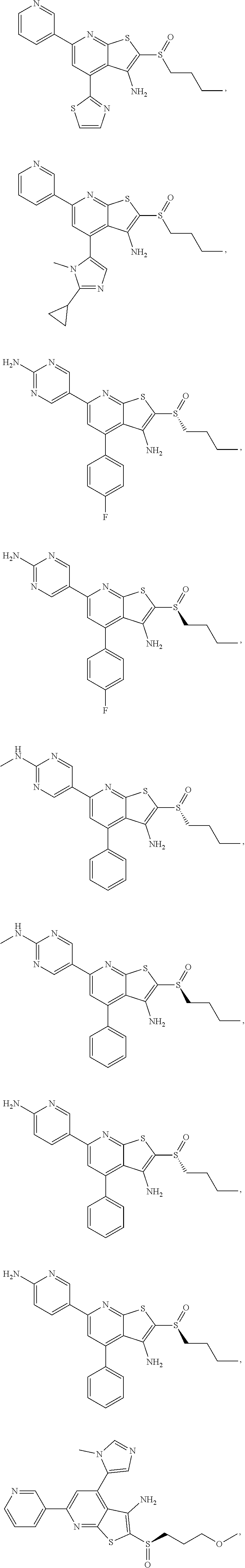

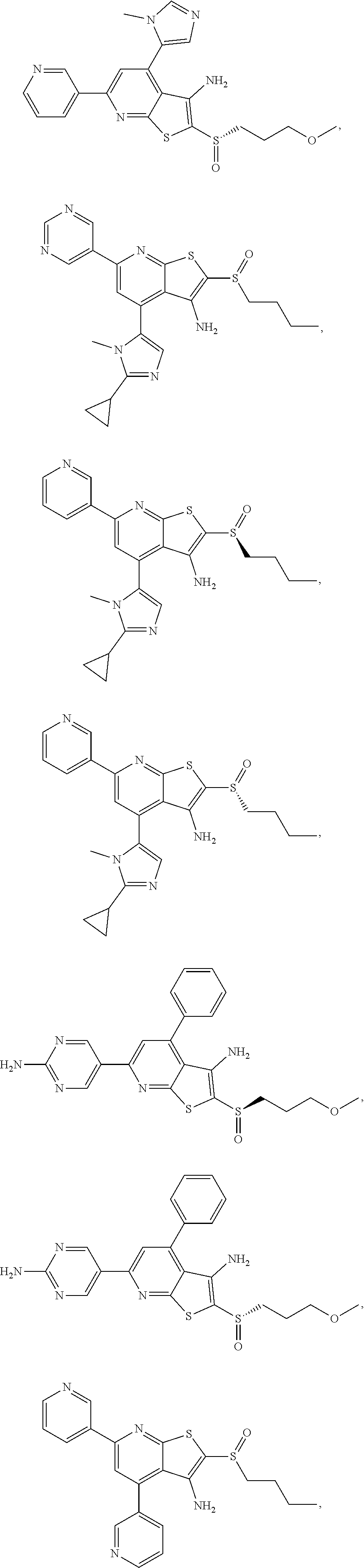

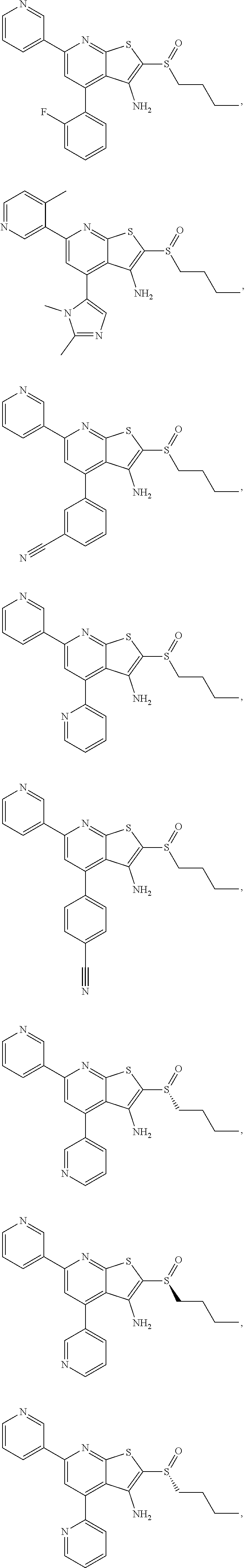

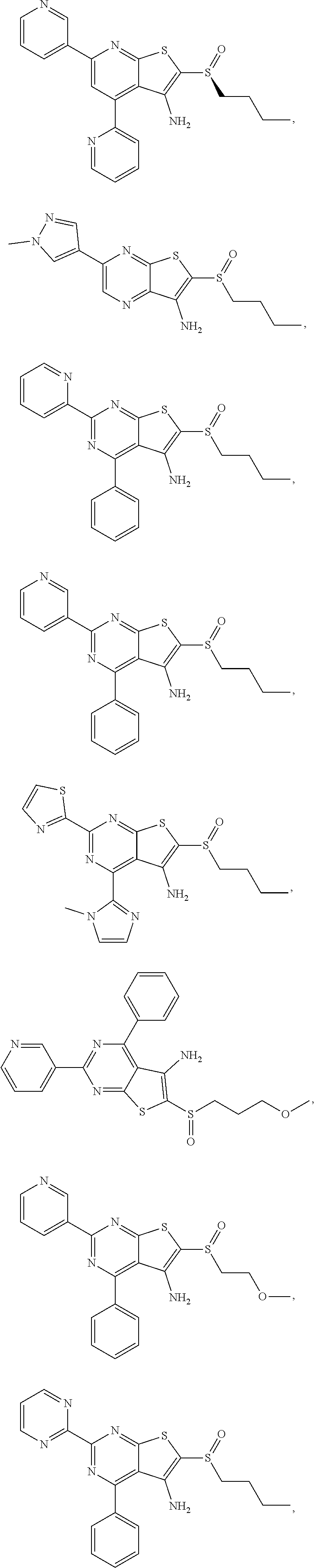

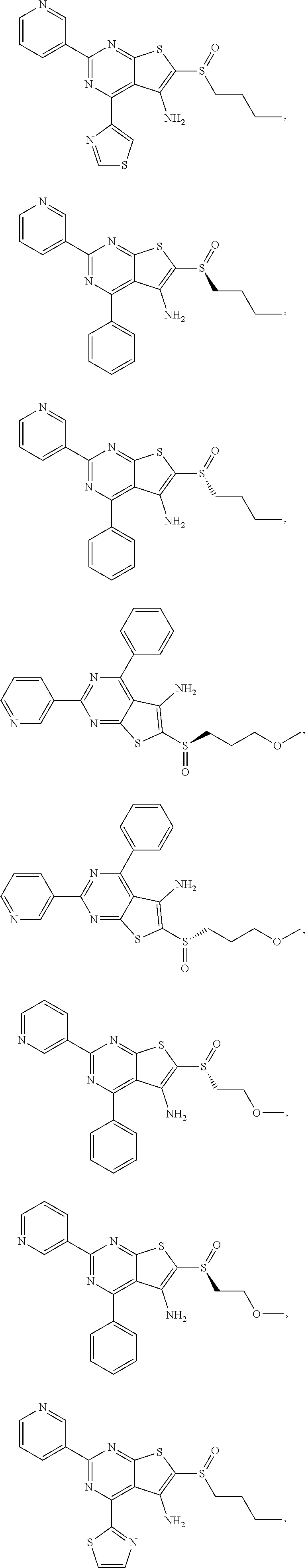

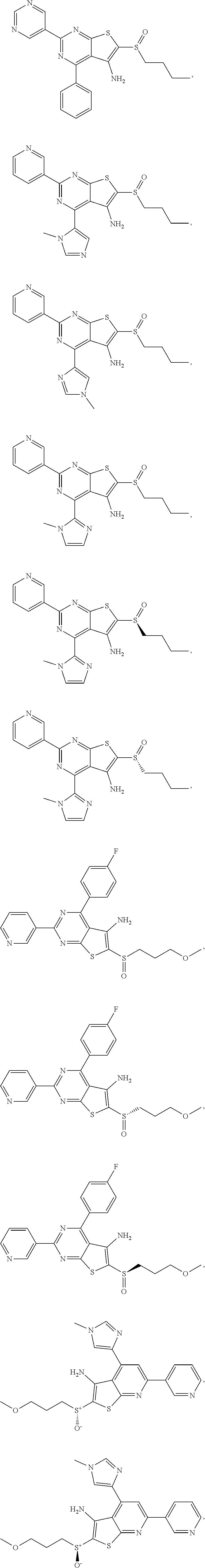

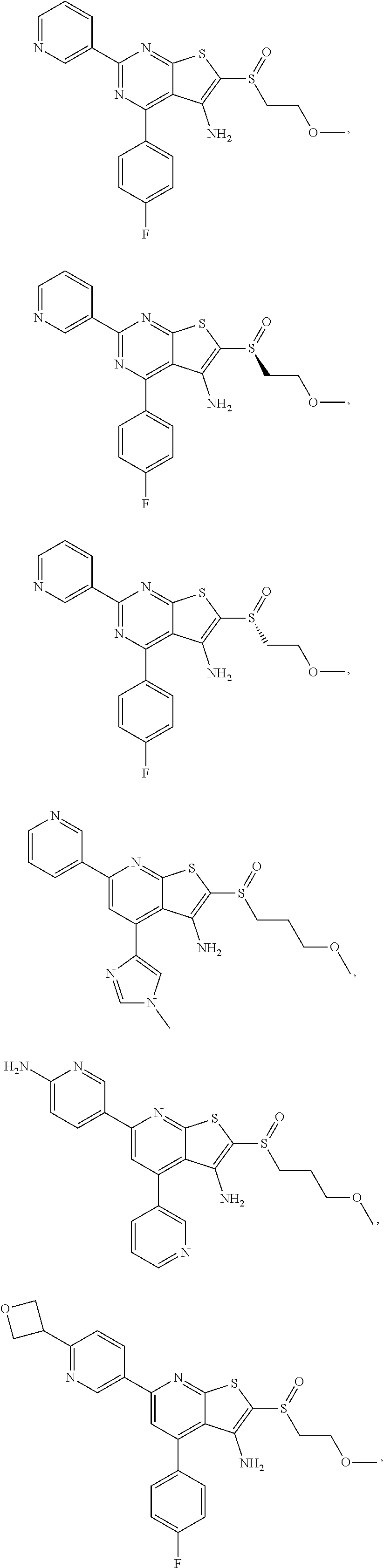 [SMILES: COCCS(=O)c1sc2nc(-c3cccnc3)nc(-c3ccc(F)cc3)c2c1N ; COCC[S@](=O)c1sc2nc(-c3cccnc3)nc(-c3ccc(F)cc3)c2c1N ; COCC[S@@](=O)c1sc2nc(-c3cccnc3)nc(-c3ccc(F)cc3)c2c1N ; COCCCS(=O)c1sc2nc(-c3cccnc3)cc(-c3cn(C)cn3)c2c1N ; COCCCS(=O)c1sc2nc(-c3ccc(N)nc3)cc(-c3cccnc3)c2c1N ; COCCS(=O)c1sc2nc(-c3ccc(C4COC4)nc3)cc(-c3ccc(F)cc3)c2c1N]

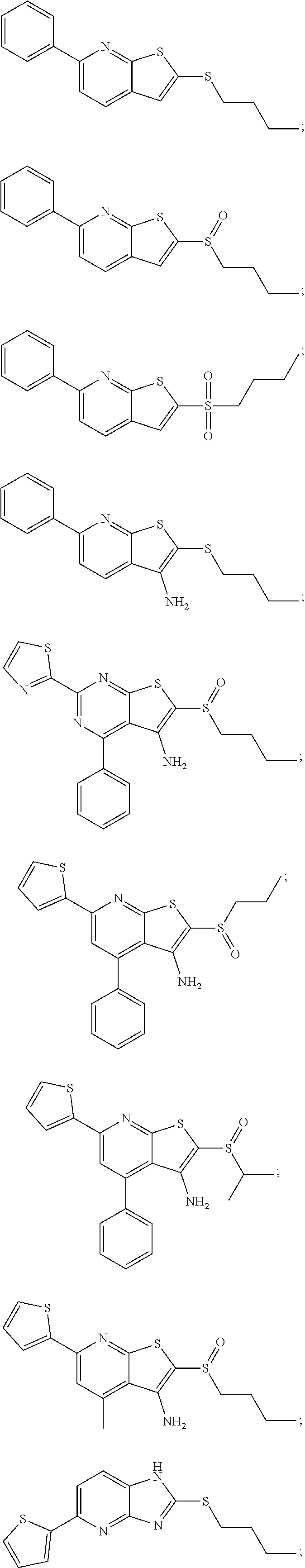

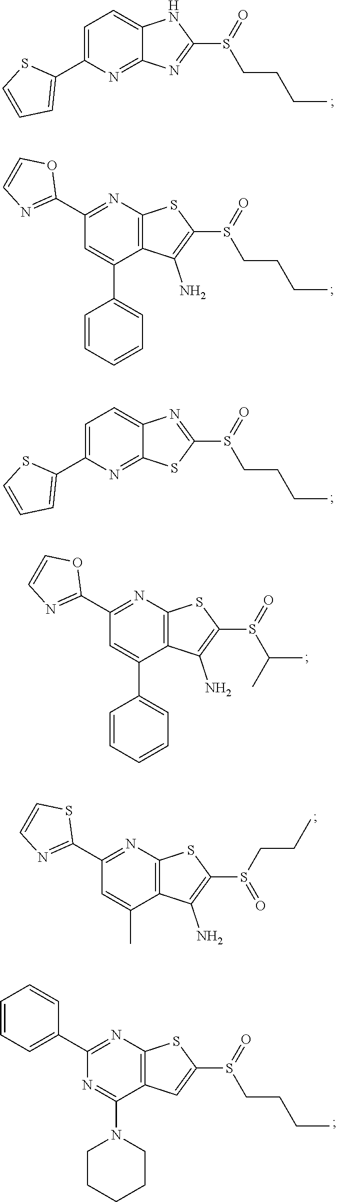

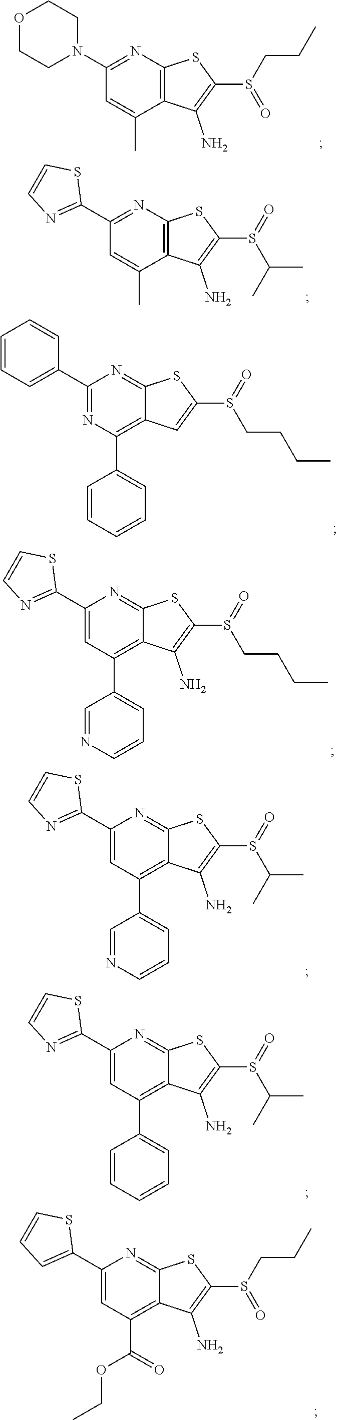

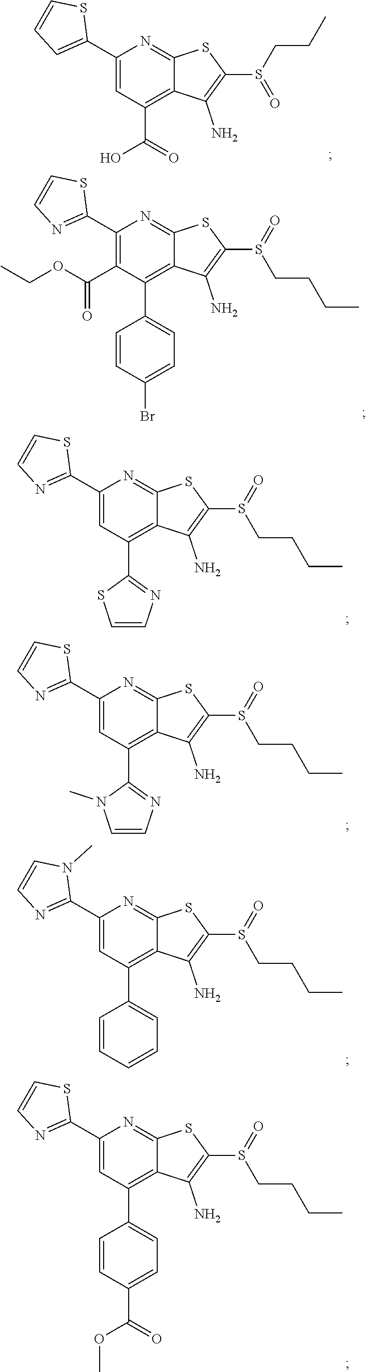

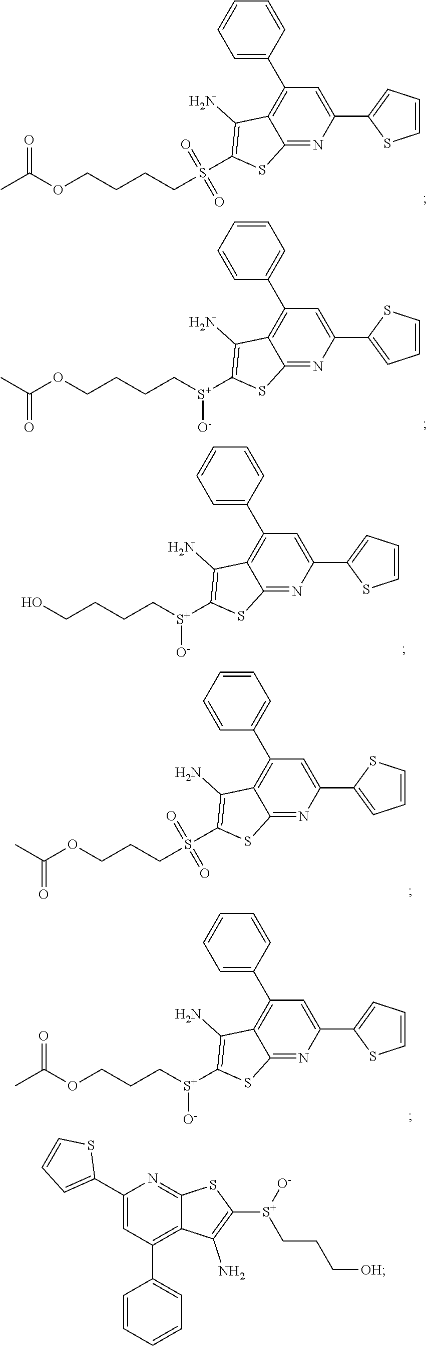

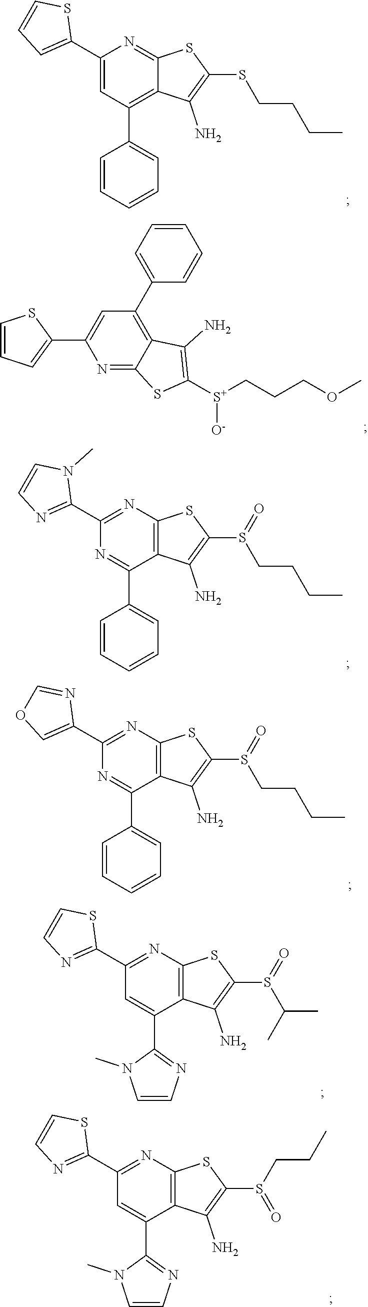

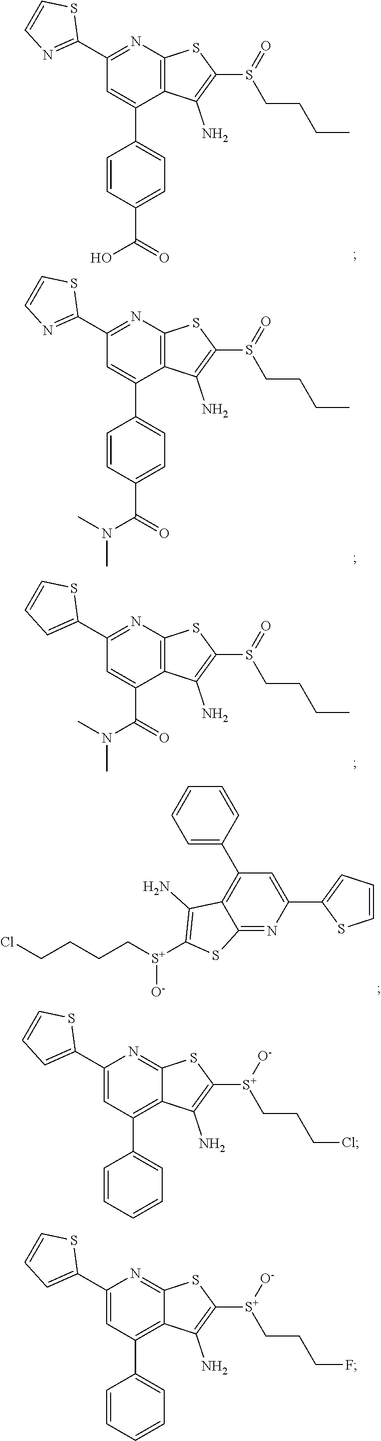

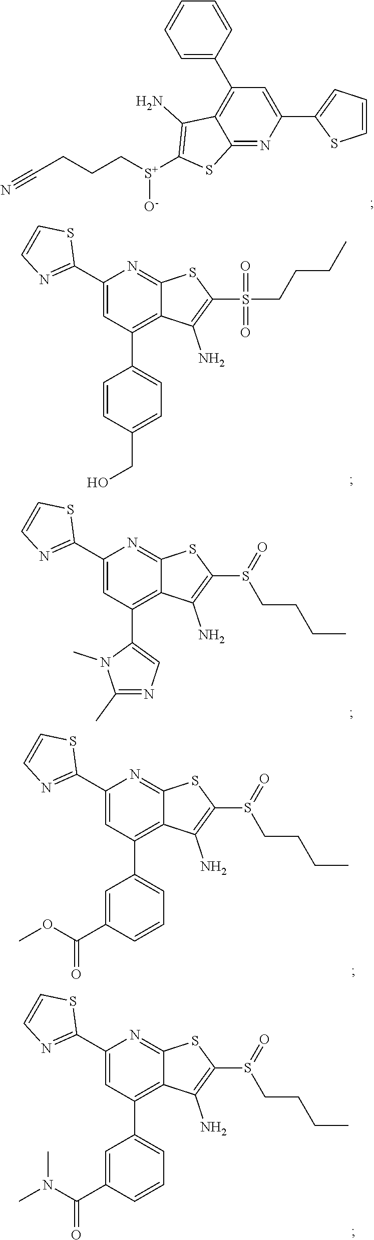

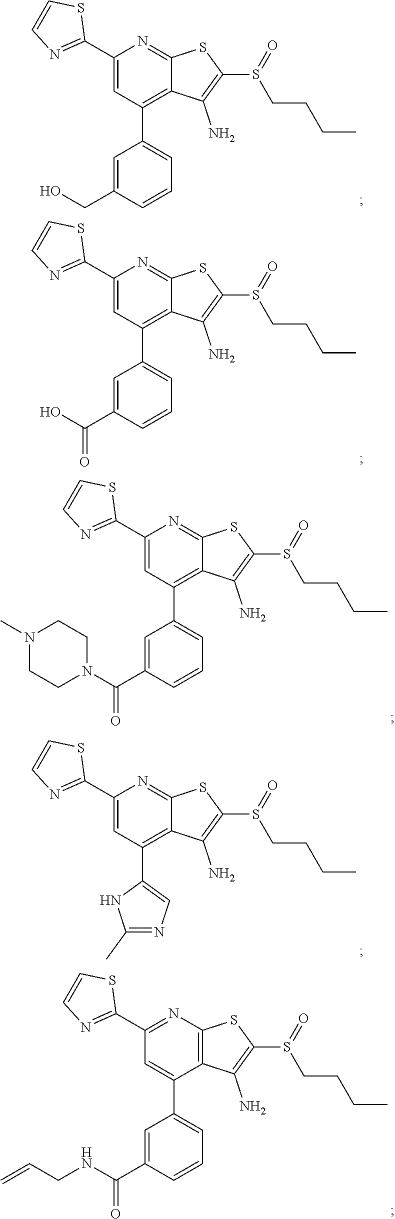

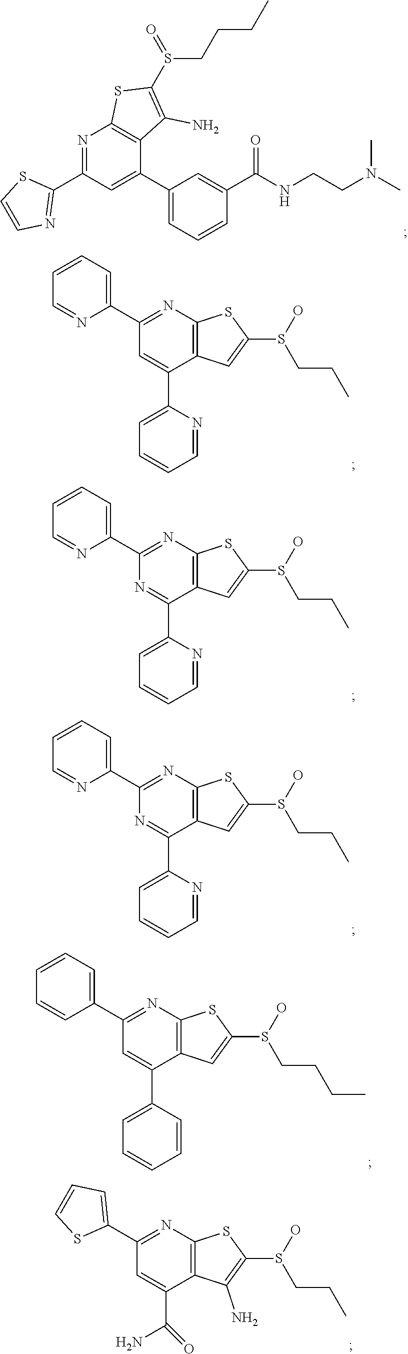

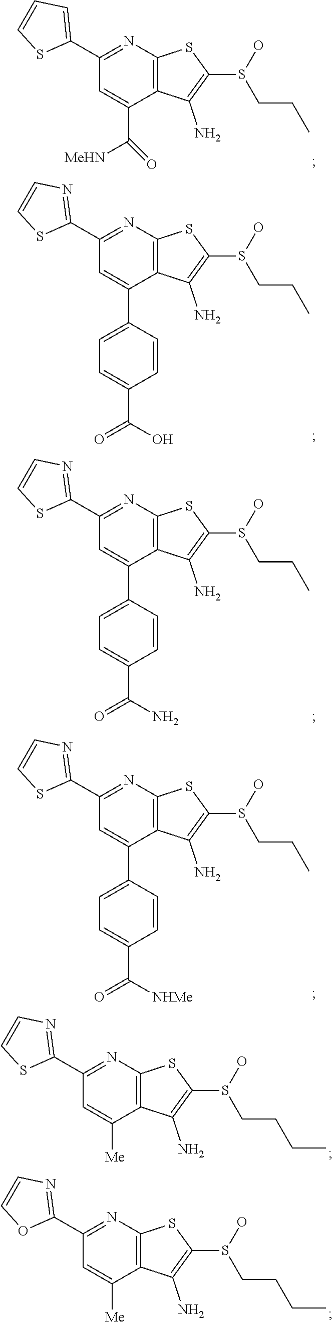

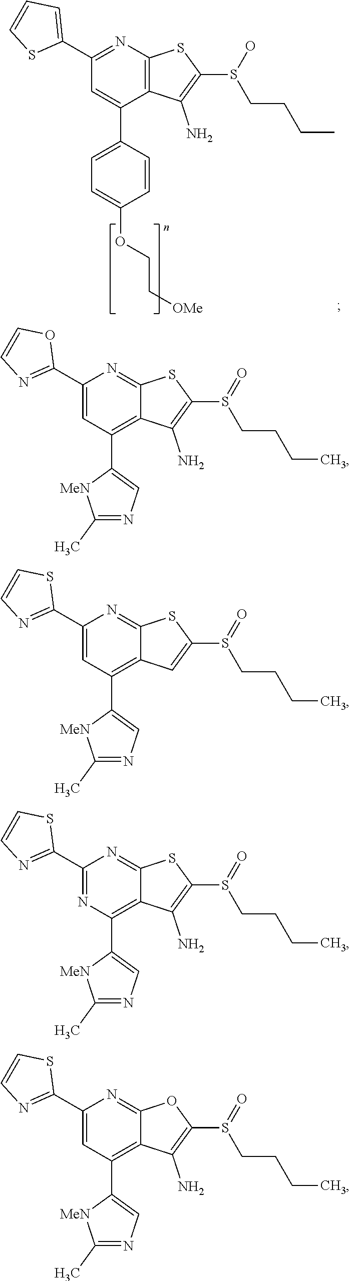

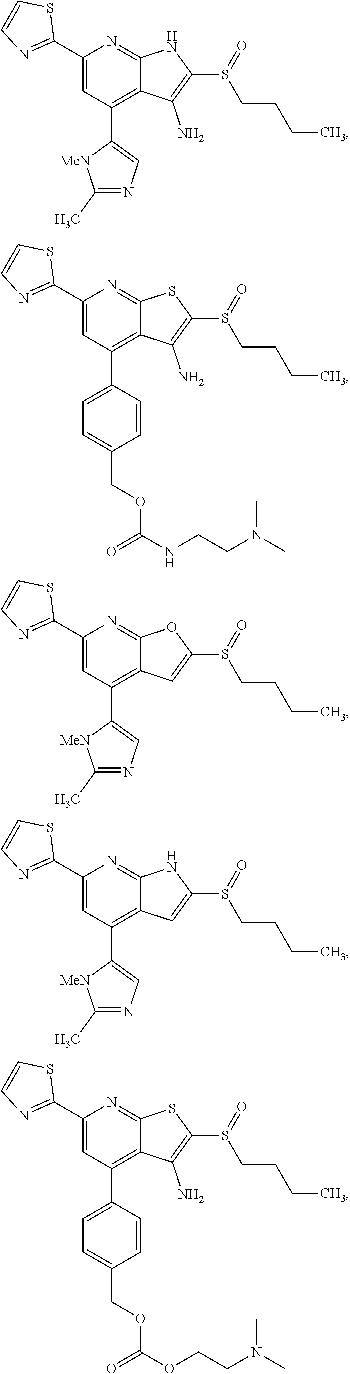

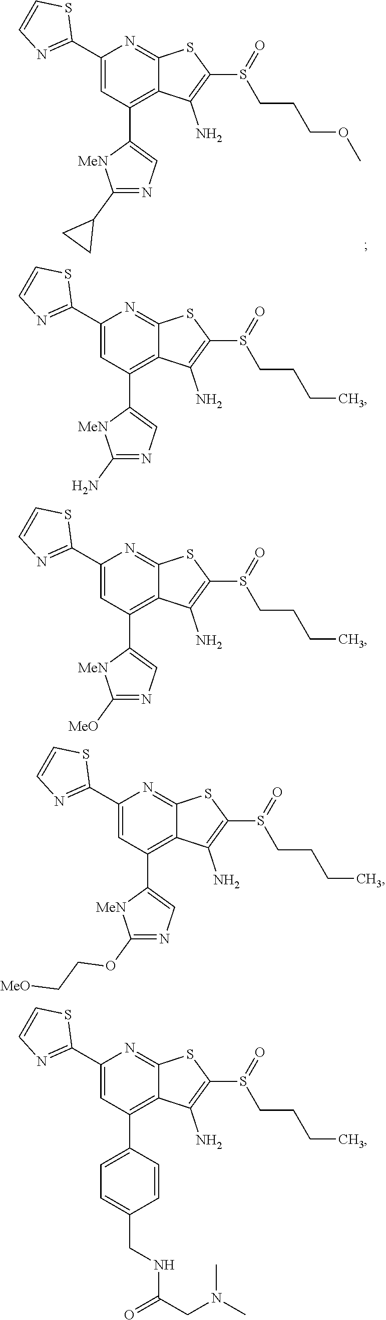

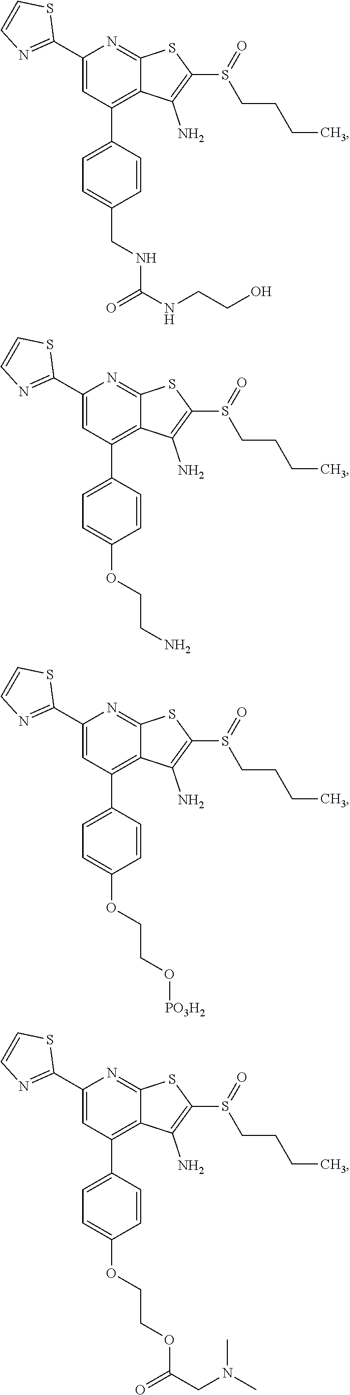

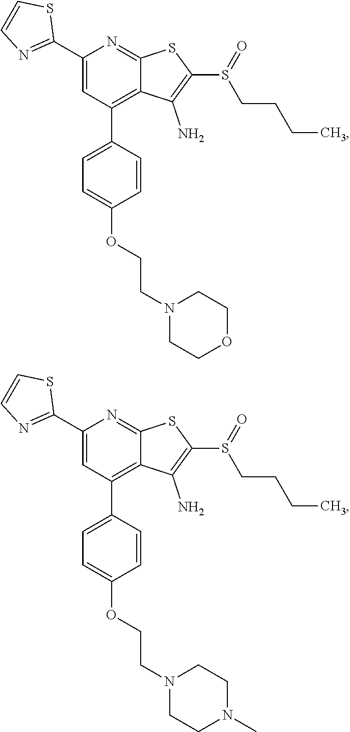 [SMILES: CCCCS(=O)c1sc2nc(-c3nccs3)cc(-c3ccc(OCCN4CCOCC4)cc3)c2c1N ; CCCCS(=O)c1sc2nc(-c3nccs3)cc(-c3ccc(OCCN4CCN(C)CC4)cc3)c2c1N]

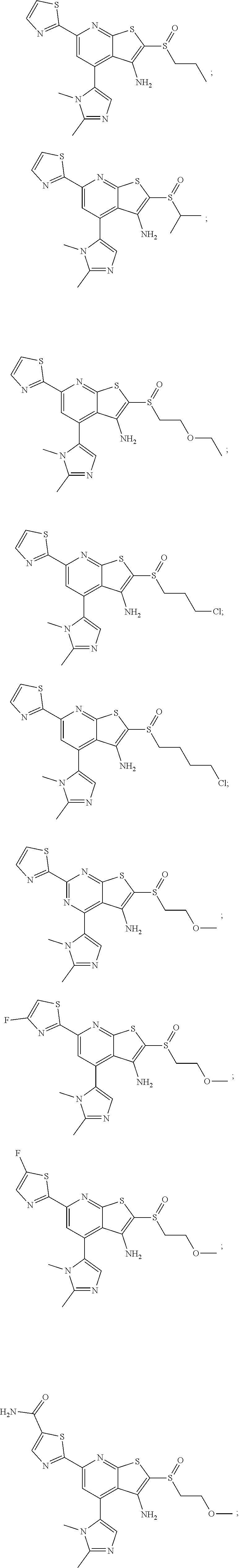

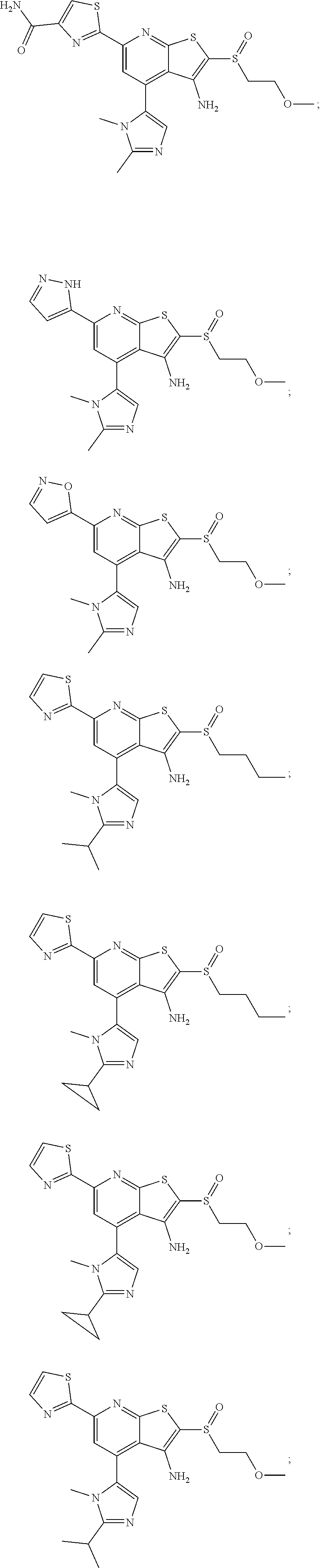

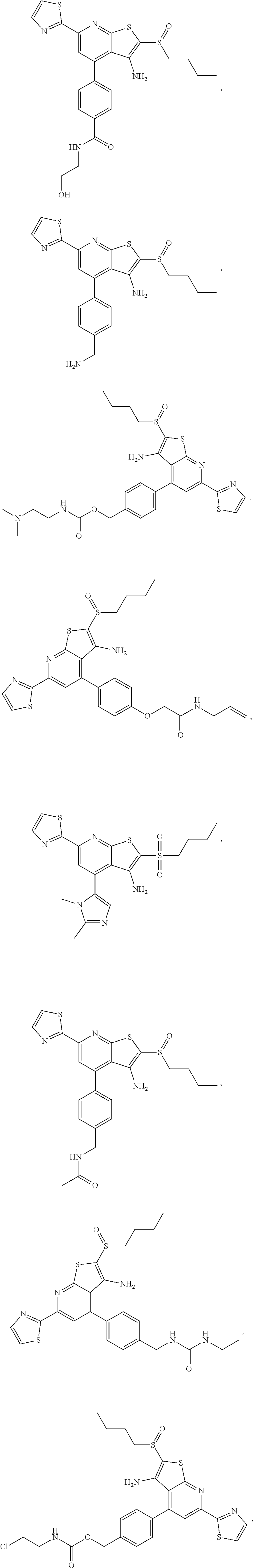

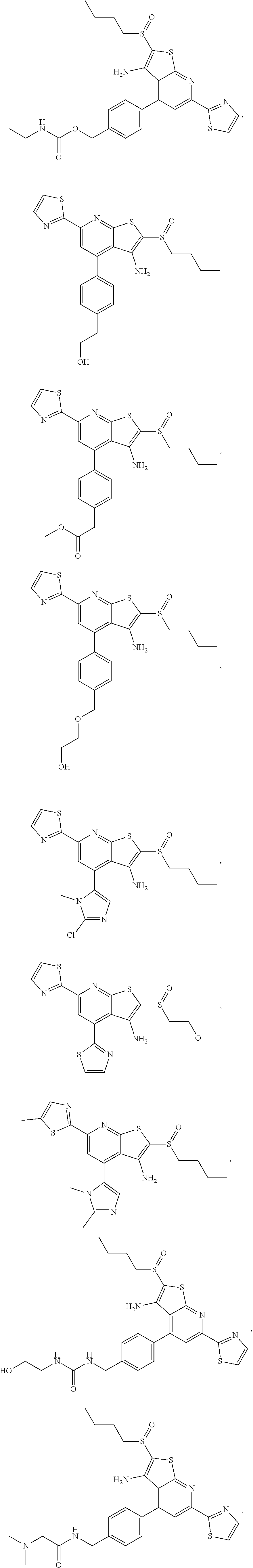

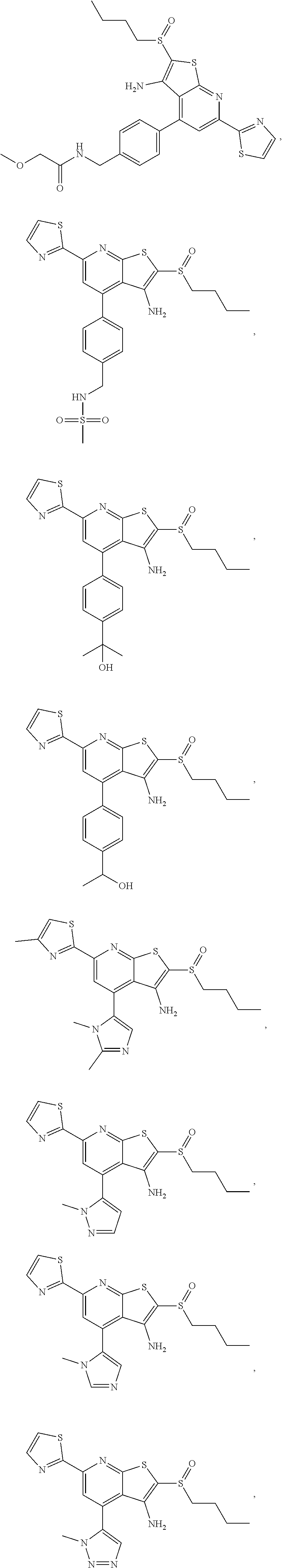

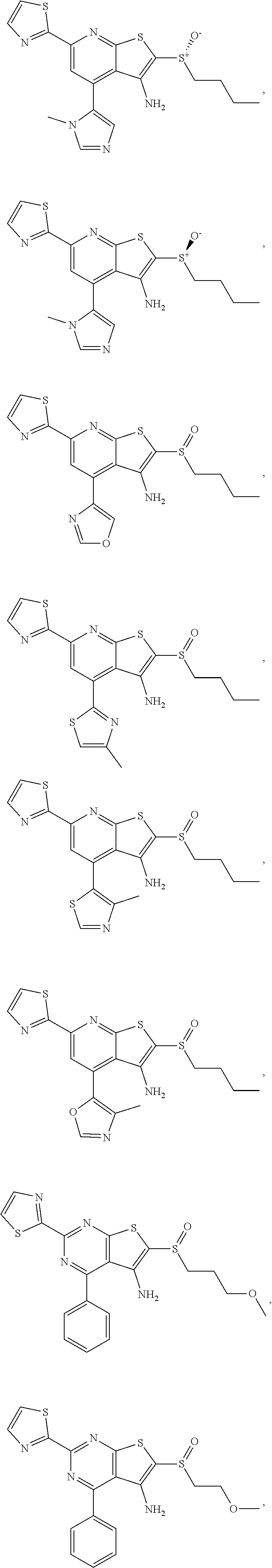

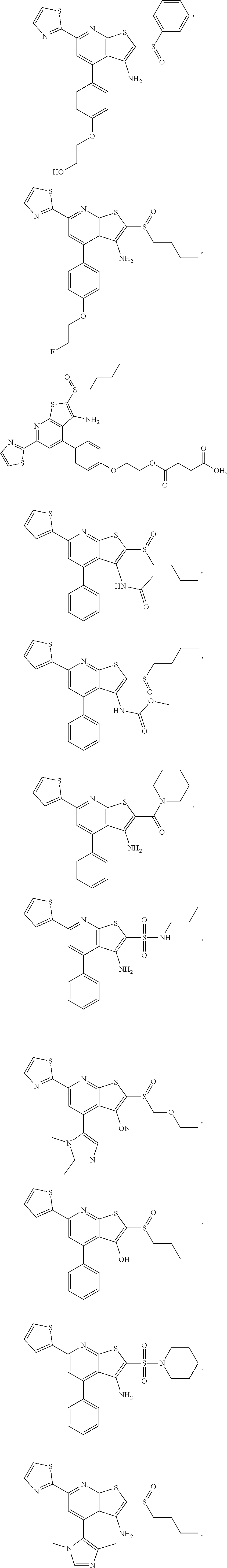

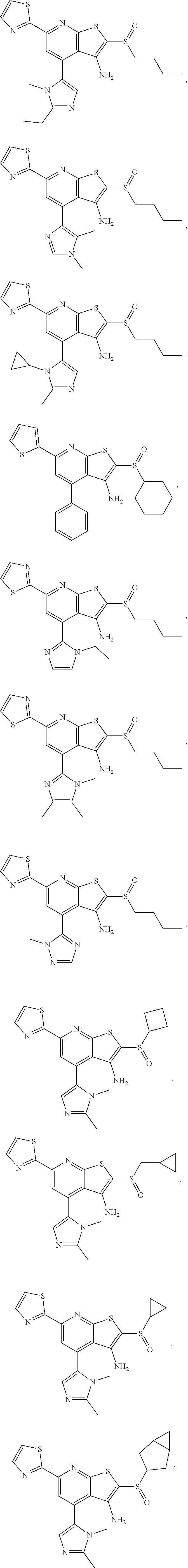

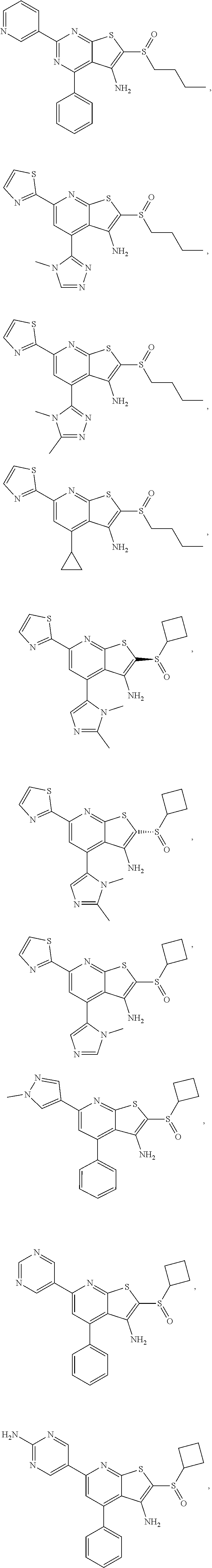

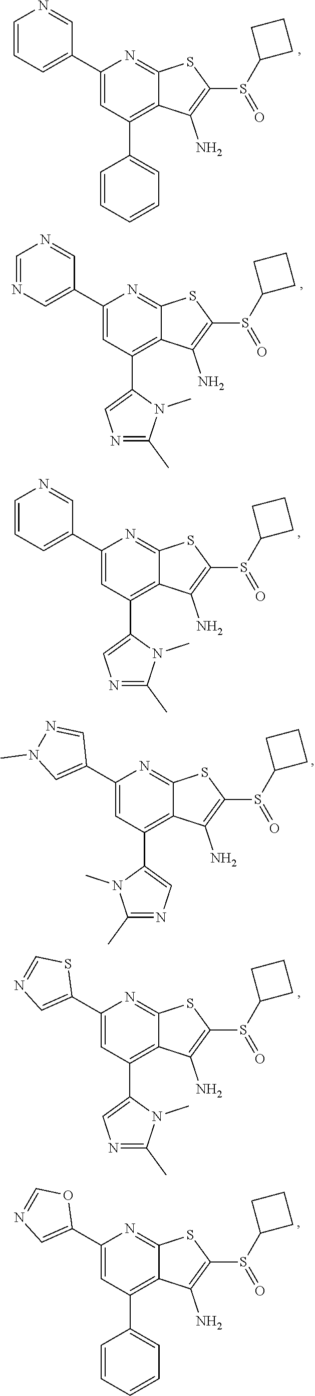

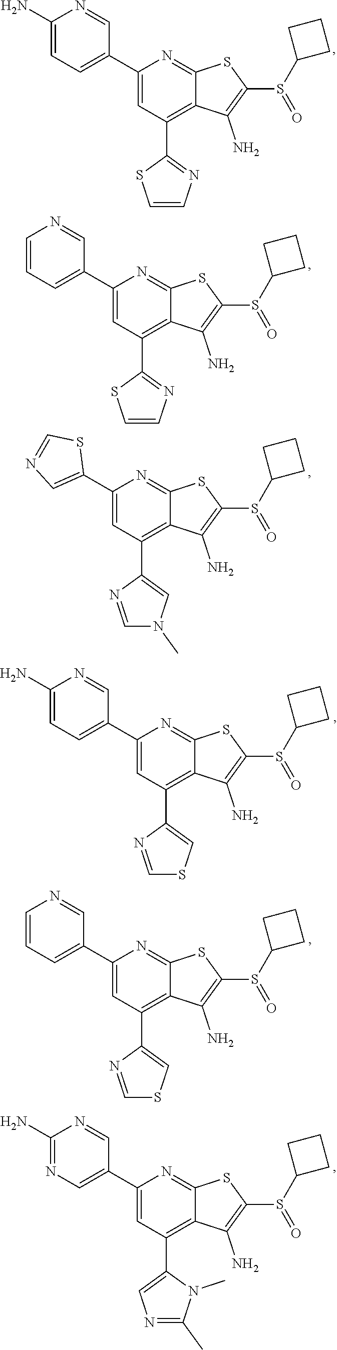

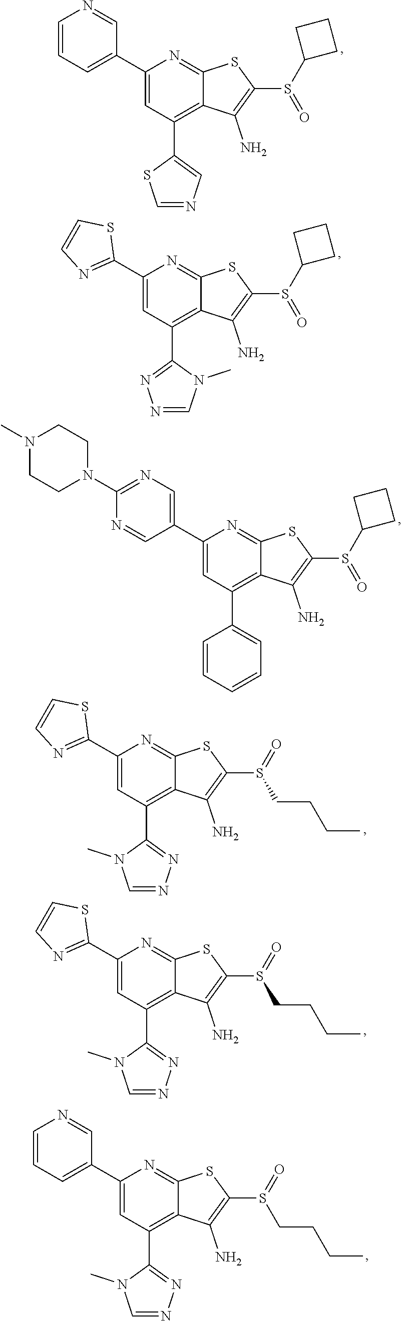 [SMILES: Nc1c(S(=O)C2CCC2)sc2nc(-c3cccnc3)cc(-c3cncs3)c12 ; Cn1cnnc1-c1cc(-c2nccs2)nc2sc(S(=O)C3CCC3)c(N)c12 ; CN1CCN(c2ncc(-c3cc(-c4ccccc4)c4c(N)c(S(=O)C5CCC5)sc4n3)cn2)CC1 ; CCCC[S@@](=O)c1sc2nc(-c3nccs3)cc(-c3nncn3C)c2c1N ; CCCC[S@](=O)c1sc2nc(-c3nccs3)cc(-c3nncn3C)c2c1N ; CCCCS(=O)c1sc2nc(-c3cccnc3)cc(-c3nncn3C)c2c1N]

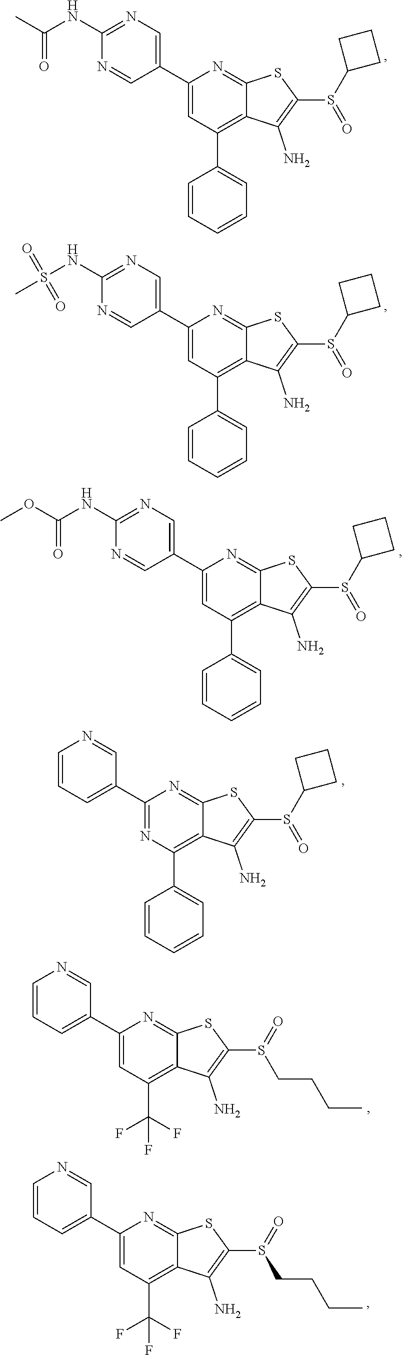 [SMILES: CC(=O)Nc1ncc(-c2cc(-c3ccccc3)c3c(N)c(S(=O)C4CCC4)sc3n2)cn1 ; CS(=O)(=O)Nc1ncc(-c2cc(-c3ccccc3)c3c(N)c(S(=O)C4CCC4)sc3n2)cn1 ; COC(=O)Nc1ncc(-c2cc(-c3ccccc3)c3c(N)c(S(=O)C4CCC4)sc3n2)cn1 ; Nc1c(S(=O)C2CCC2)sc2nc(-c3cccnc3)nc(-c3ccccc3)c12 ; CCCCS(=O)c1sc2nc(-c3cccnc3)cc(C(F)(F)F)c2c1N ; CCCC[S@](=O)c1sc2nc(-c3cccnc3)cc(C(F)(F)F)c2c1N]

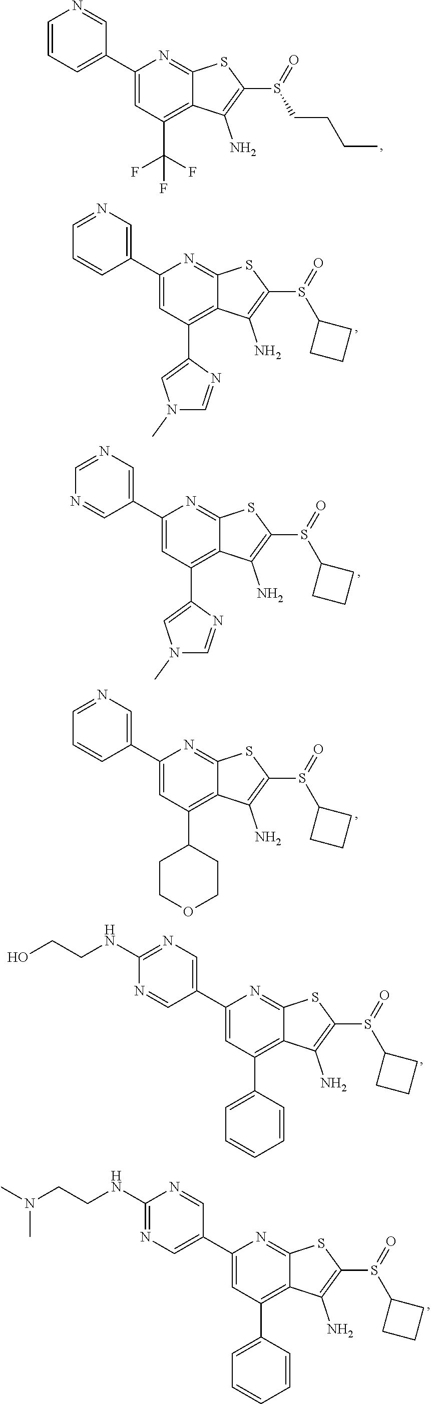

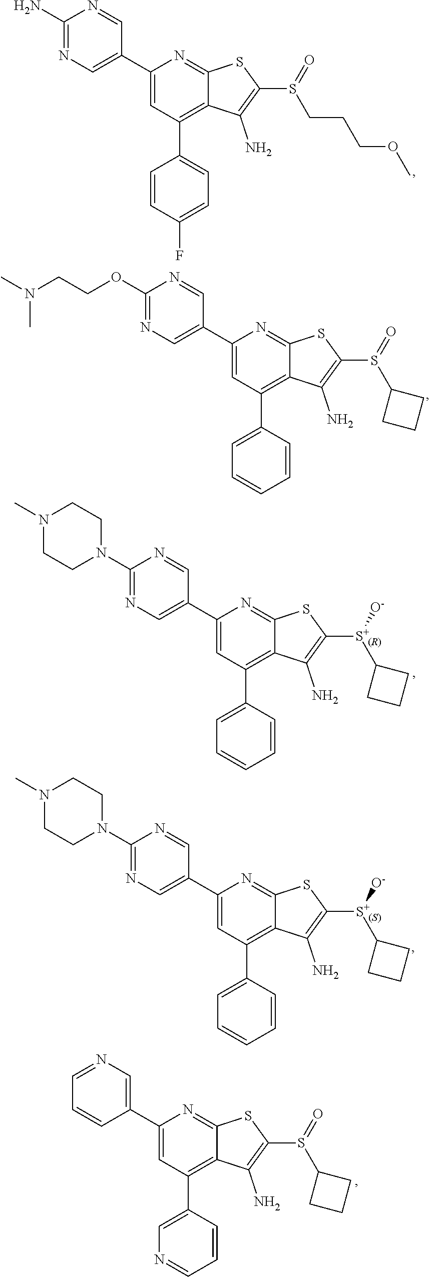

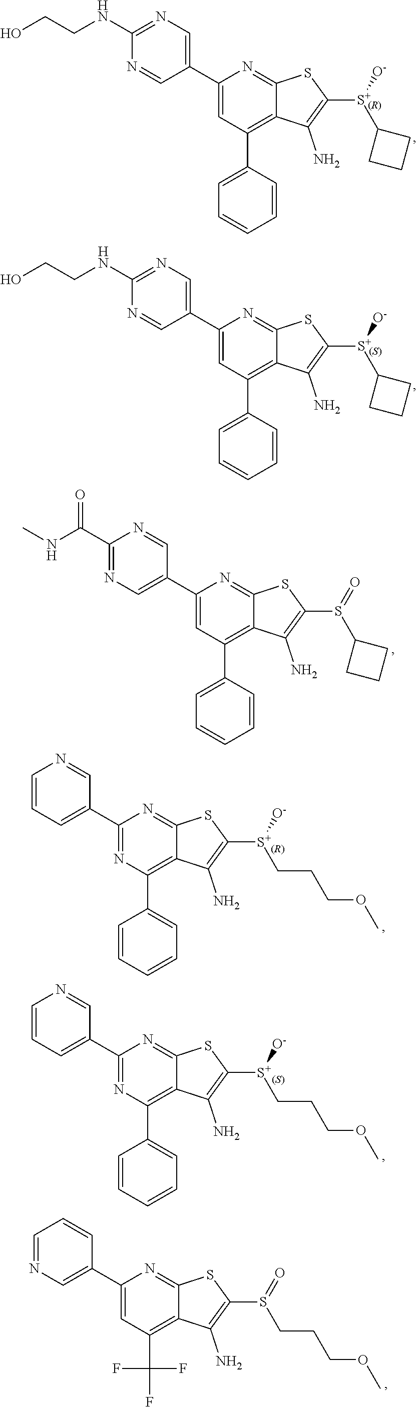

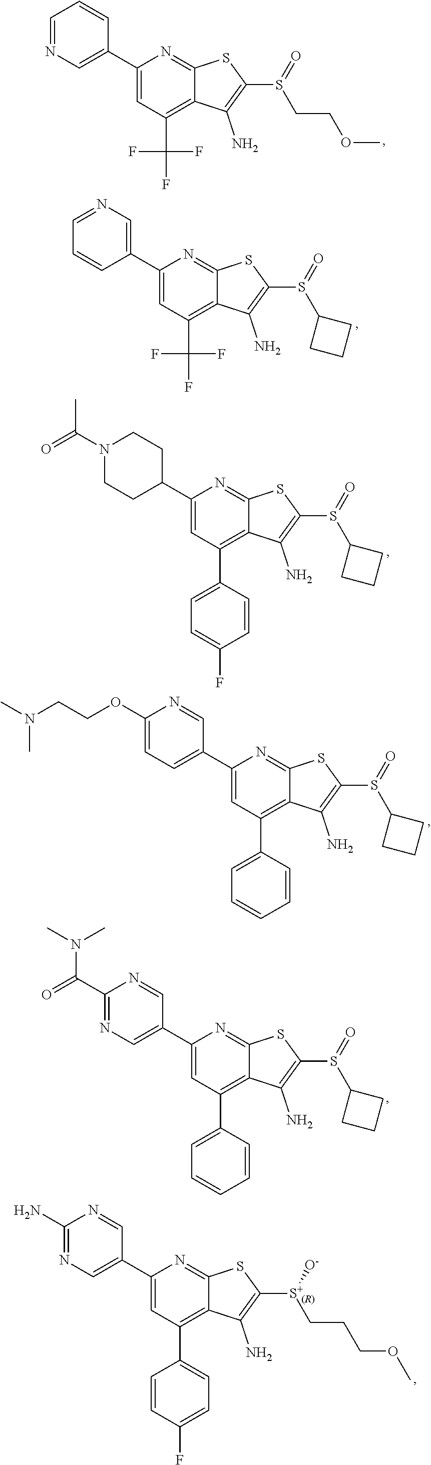

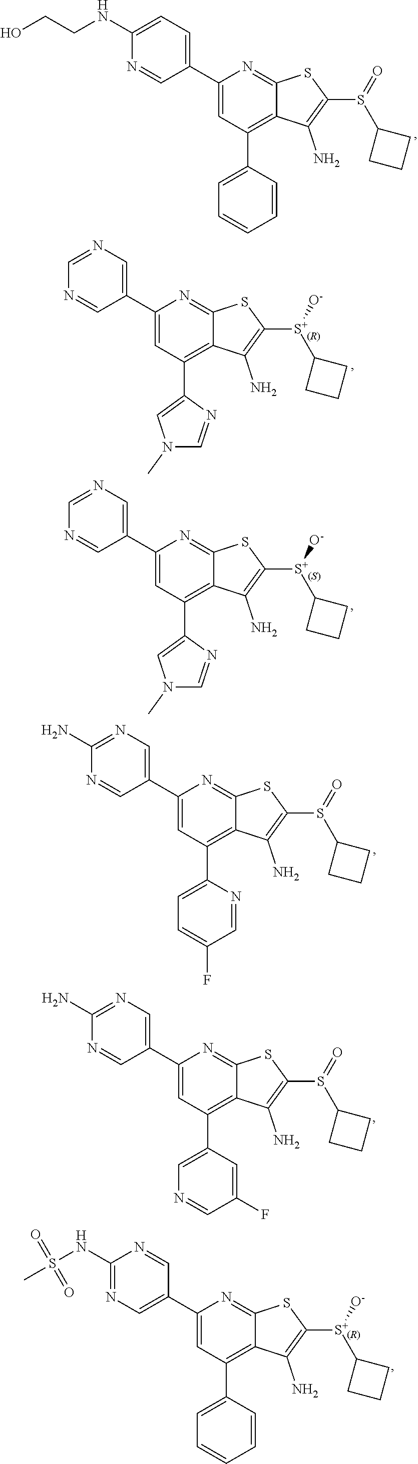

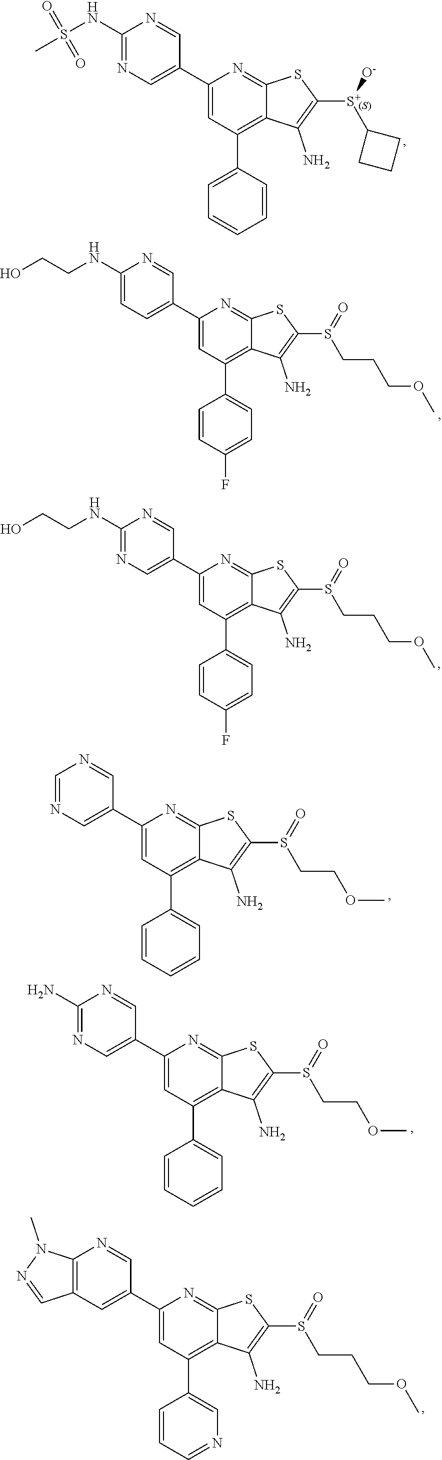

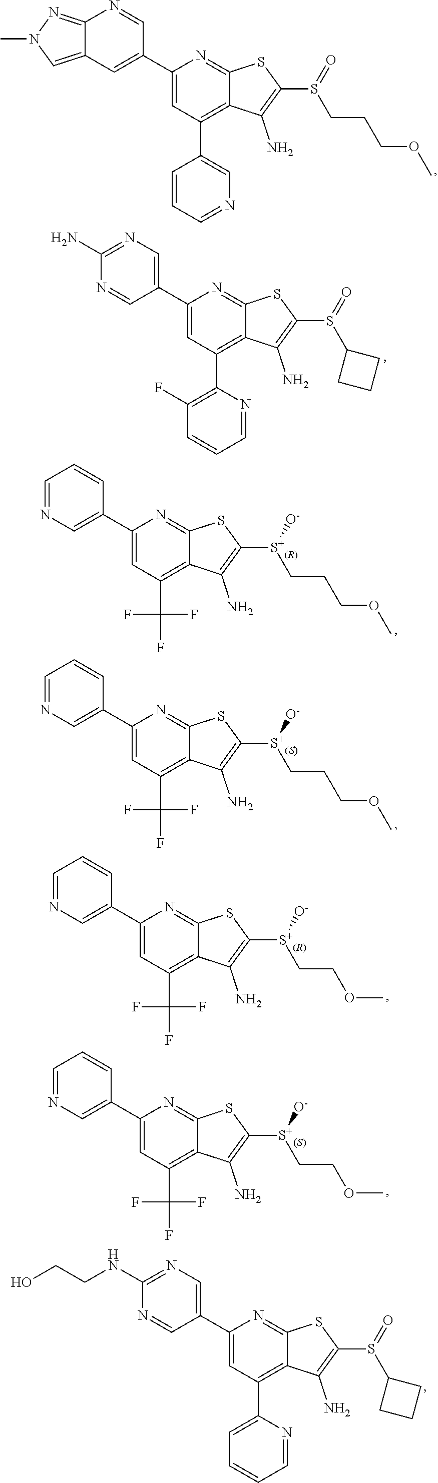

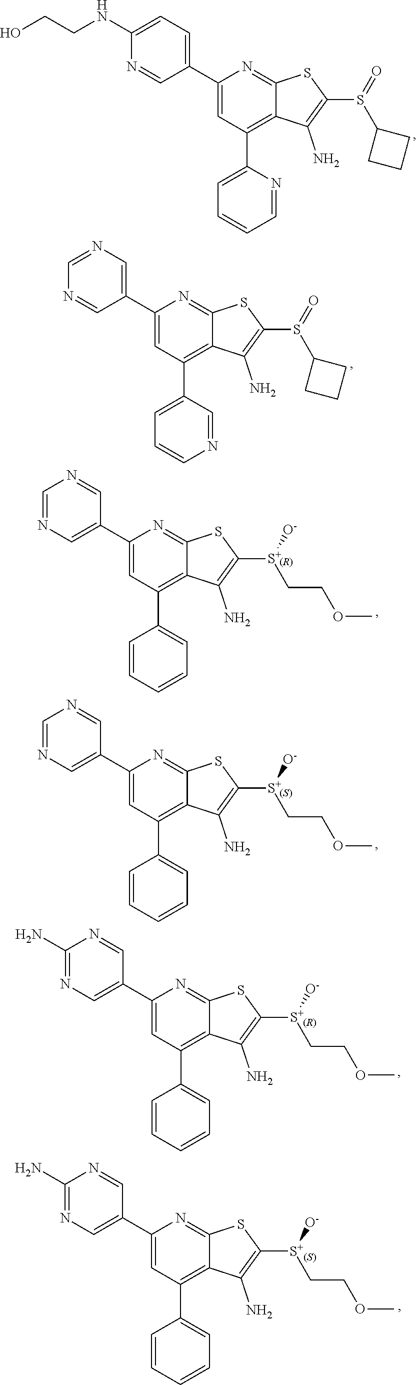

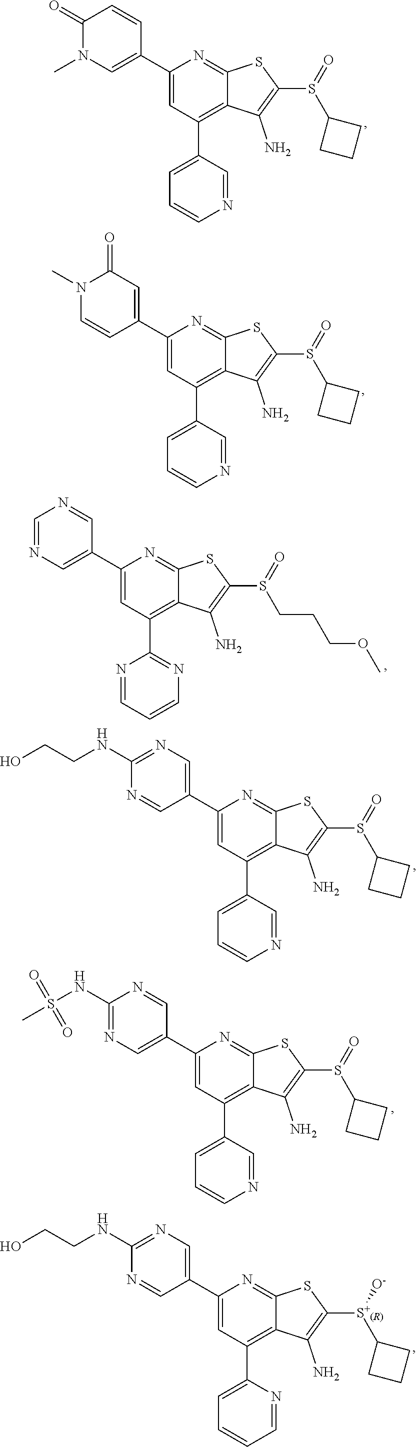

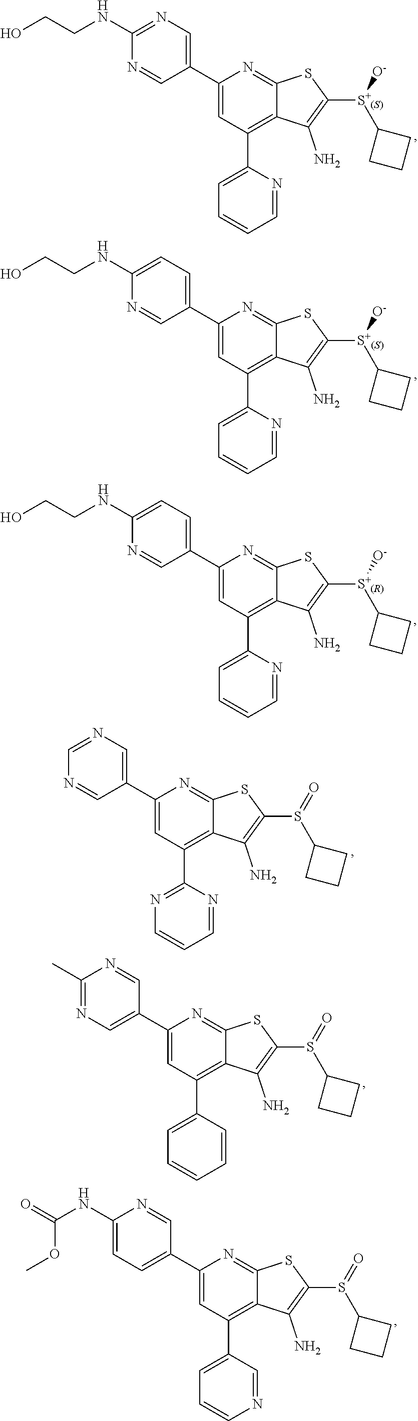

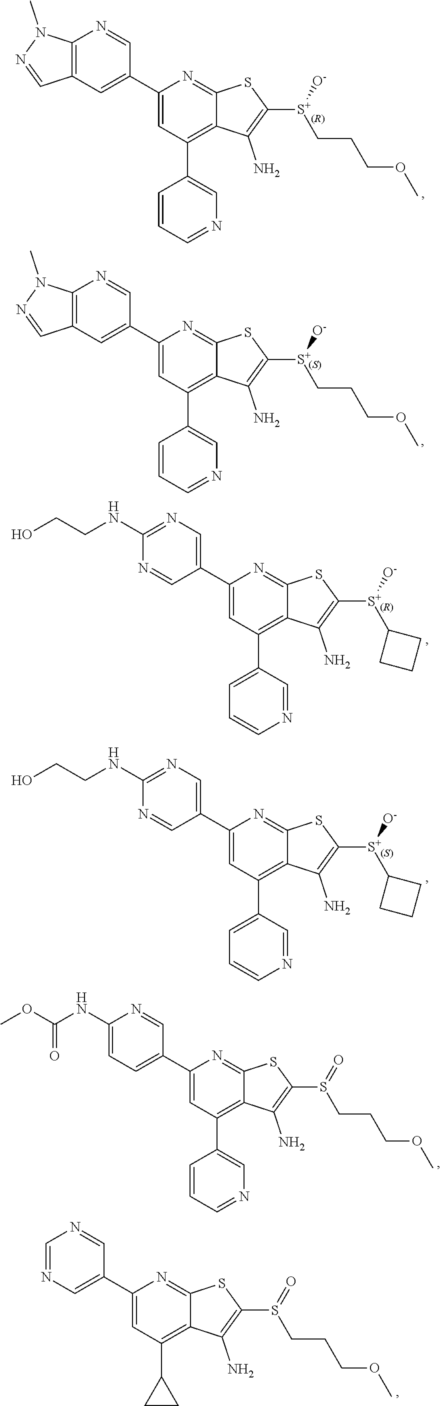

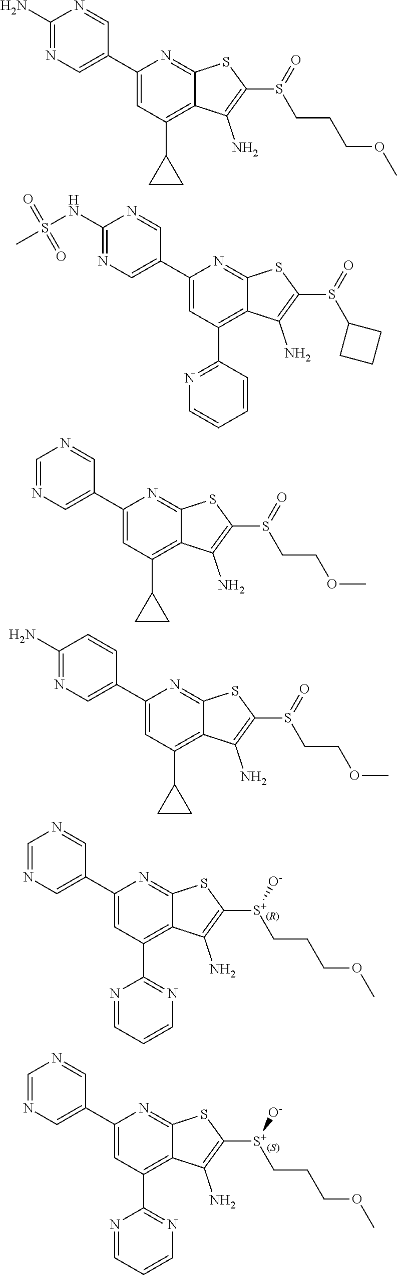

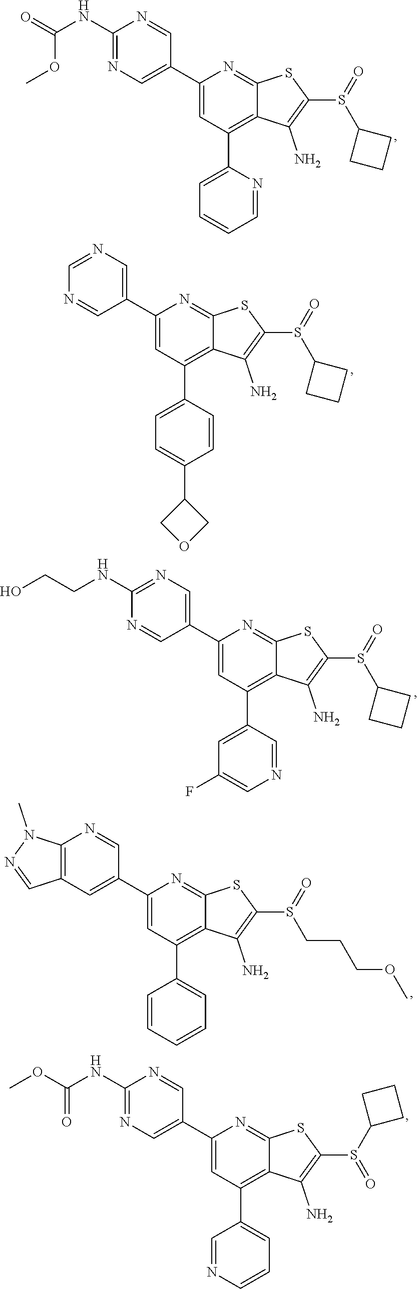

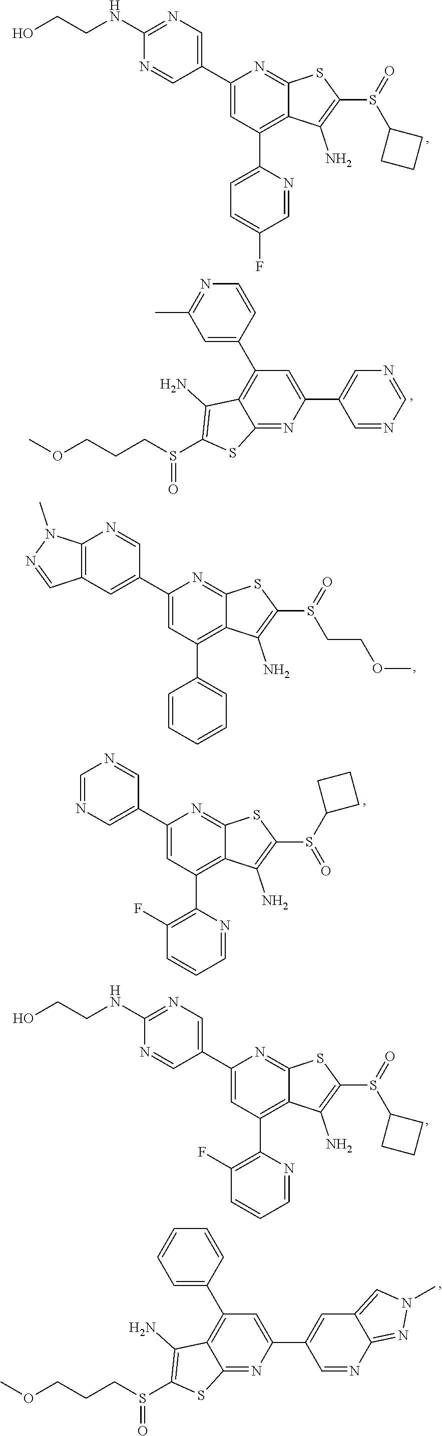

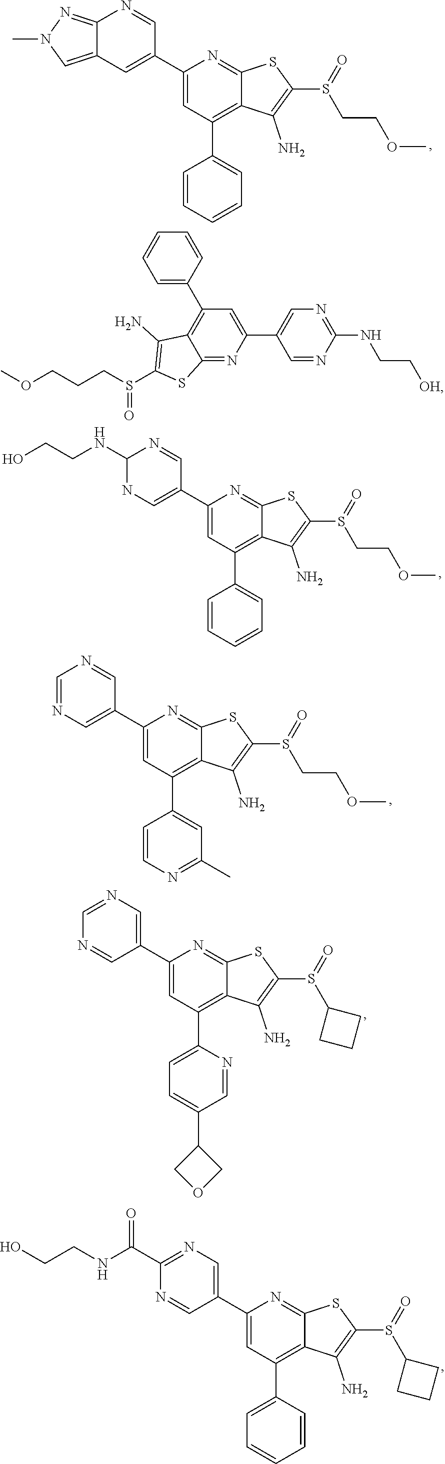

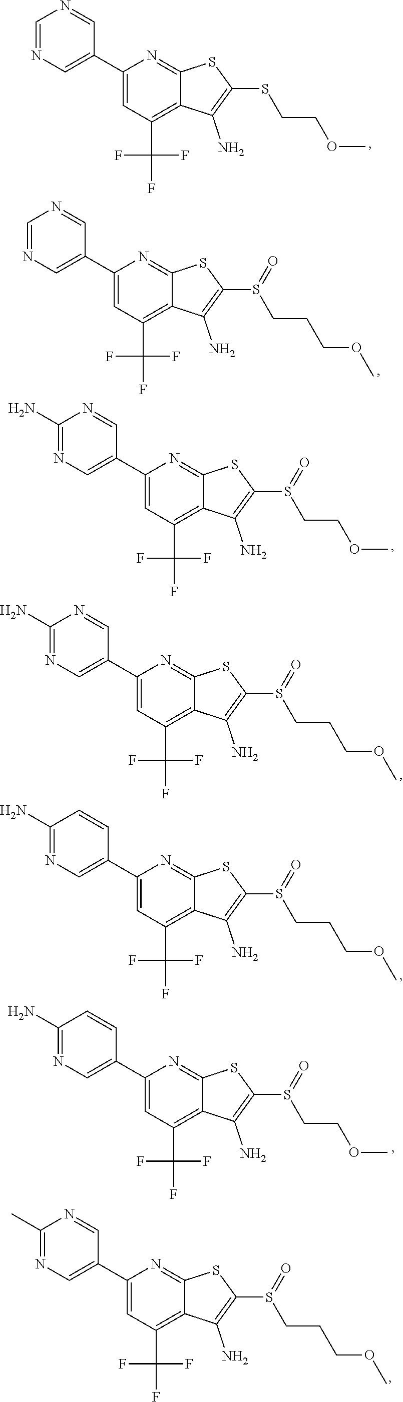

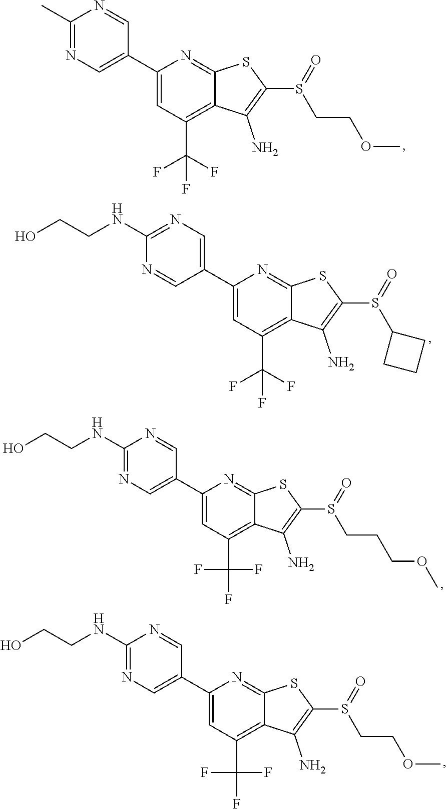

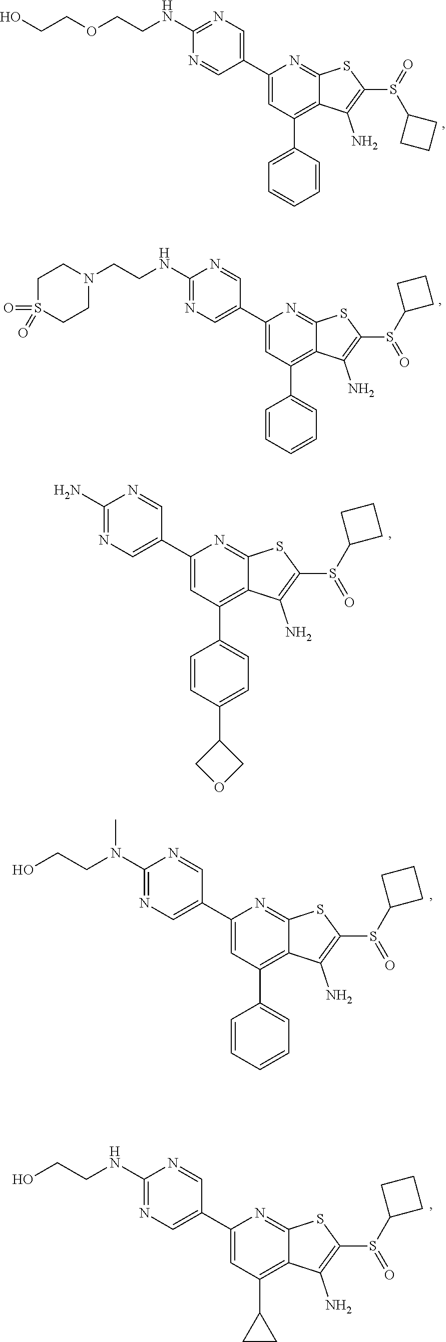 [SMILES: Nc1c(S(=O)C2CCC2)sc2nc(-c3cnc(NCCOCCO)nc3)cc(-c3ccccc3)c12 ; Nc1c(S(=O)C2CCC2)sc2nc(-c3cnc(NCCN4CCS(=O)(=O)CC4)nc3)cc(-c3ccccc3)c12 ; Nc1ncc(-c2cc(-c3ccc(C4COC4)cc3)c3c(N)c(S(=O)C4CCC4)sc3n2)cn1 ; CN(CCO)c1ncc(-c2cc(-c3ccccc3)c3c(N)c(S(=O)C4CCC4)sc3n2)cn1 ; Nc1c(S(=O)C2CCC2)sc2nc(-c3cnc(NCCO)nc3)cc(C3CC3)c12]

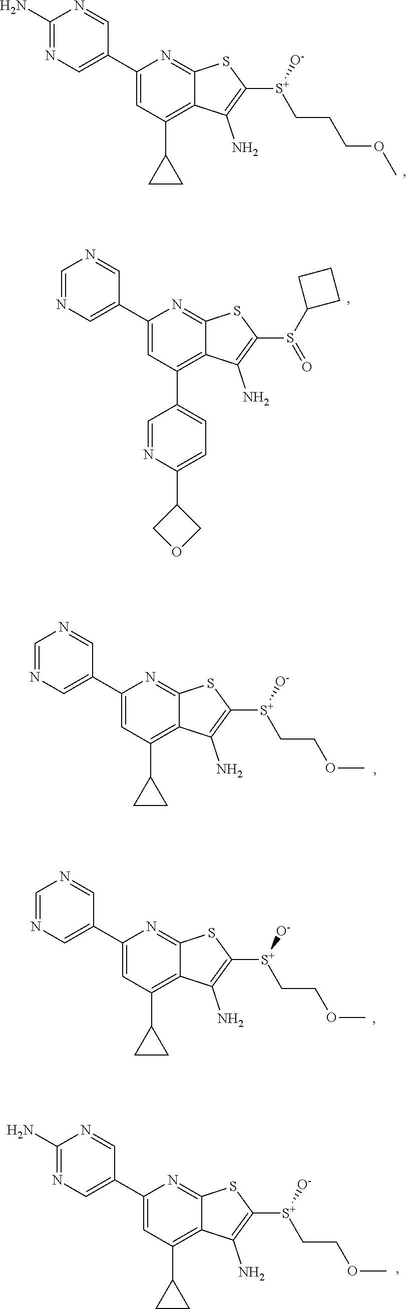

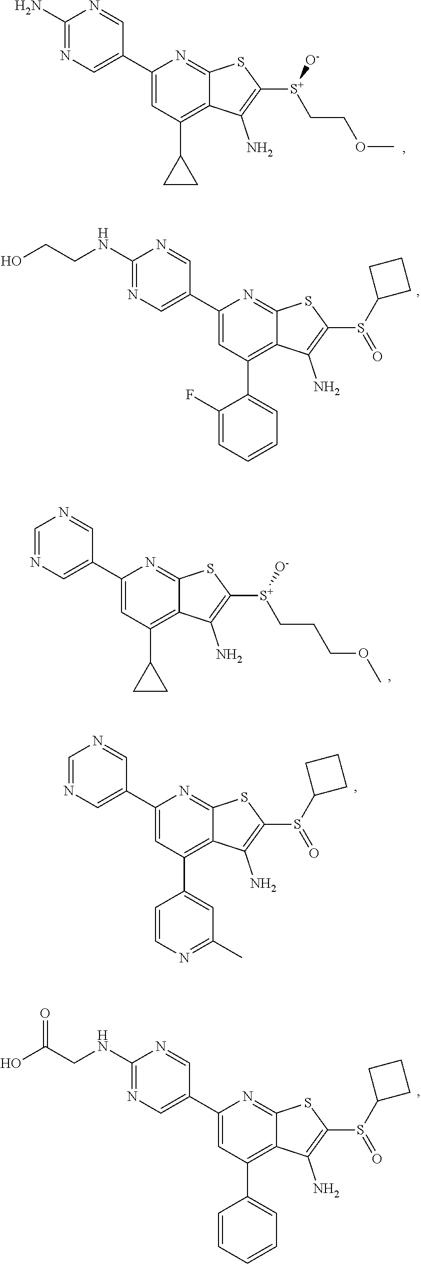

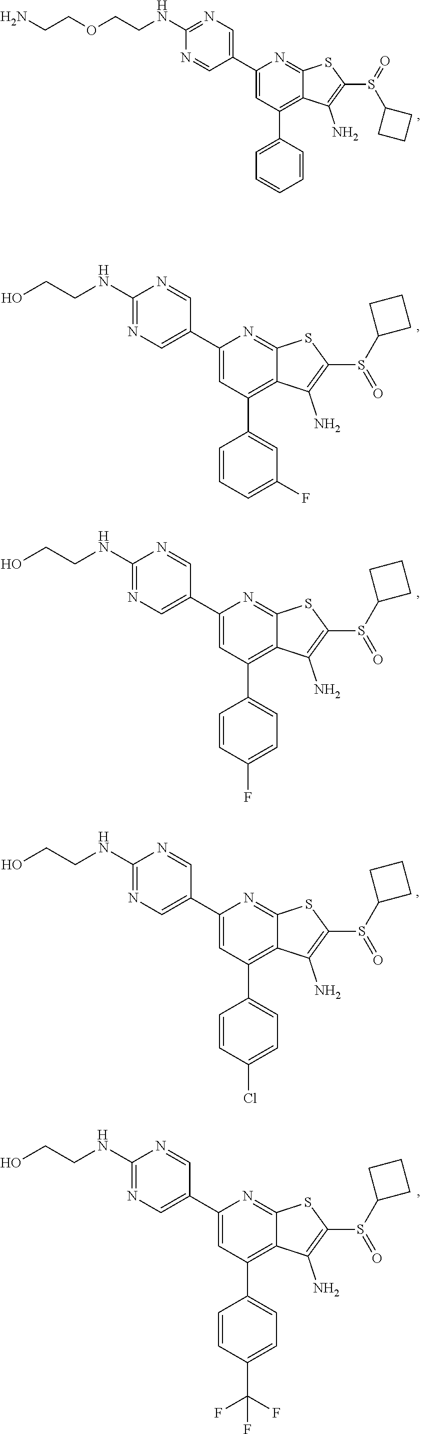 [SMILES: NCCOCCNc1ncc(-c2cc(-c3ccccc3)c3c(N)c(S(=O)C4CCC4)sc3n2)cn1 ; Nc1c(S(=O)C2CCC2)sc2nc(-c3cnc(NCCO)nc3)cc(-c3cccc(F)c3)c12 ; Nc1c(S(=O)C2CCC2)sc2nc(-c3cnc(NCCO)nc3)cc(-c3ccc(F)cc3)c12 ; Nc1c(S(=O)C2CCC2)sc2nc(-c3cnc(NCCO)nc3)cc(-c3ccc(Cl)cc3)c12 ; Nc1c(S(=O)C2CCC2)sc2nc(-c3cnc(NCCO)nc3)cc(-c3ccc(C(F)(F)F)cc3)c12]

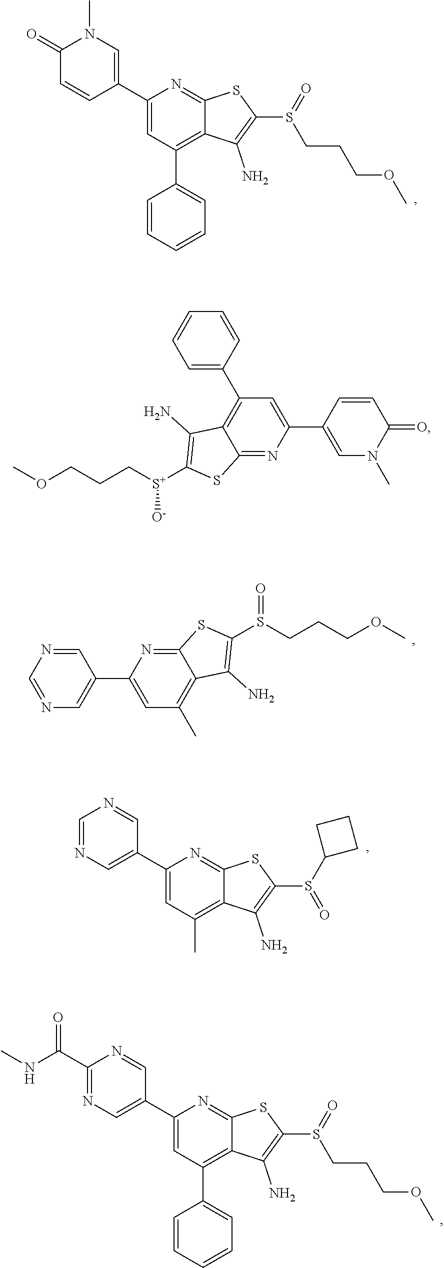

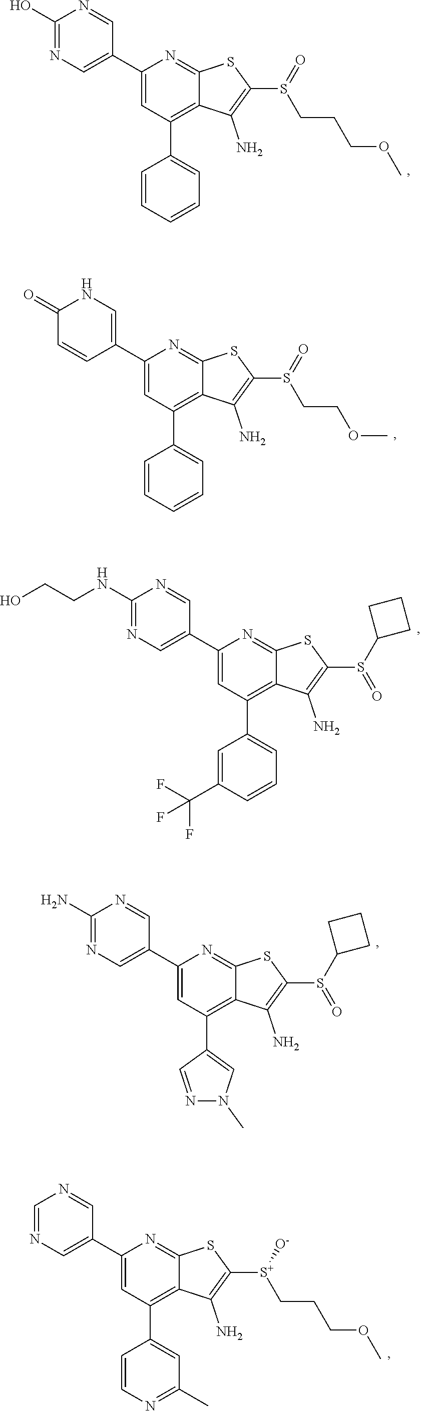 [SMILES: COCCCS(=O)c1sc2nc(-c3cnc(O)nc3)cc(-c3ccccc3)c2c1N ; COCCS(=O)c1sc2nc(-c3ccc(=O)[nH]c3)cc(-c3ccccc3)c2c1N ; Nc1c(S(=O)C2CCC2)sc2nc(-c3cnc(NCCO)nc3)cc(-c3cccc(C(F)(F)F)c3)c12 ; Cn1cc(-c2cc(-c3cnc(N)nc3)nc3sc(S(=O)C4CCC4)c(N)c23)cn1 ; COCCC[S@@+]([O-])c1sc2nc(-c3cncnc3)cc(-c3ccnc(C)c3)c2c1N]

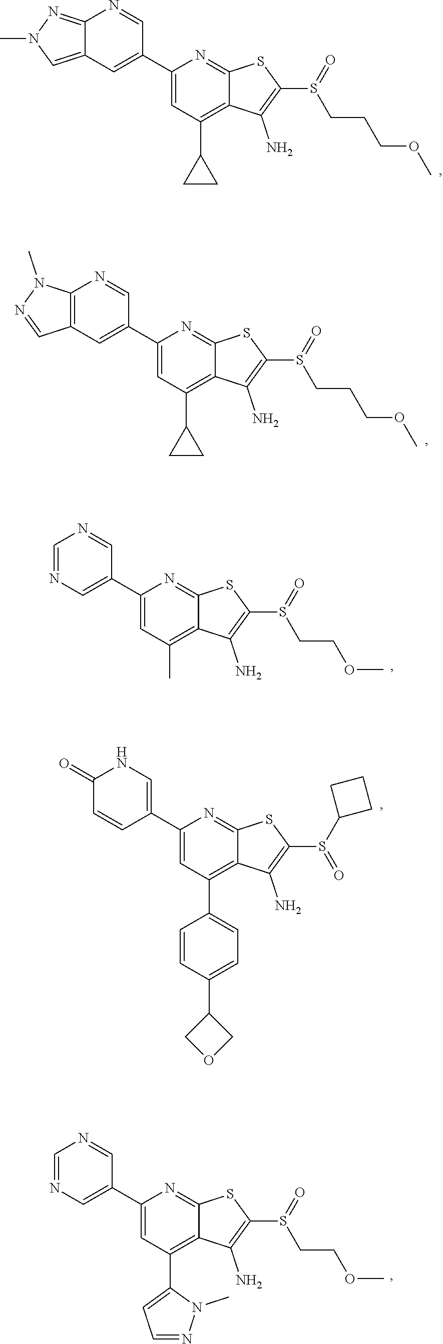

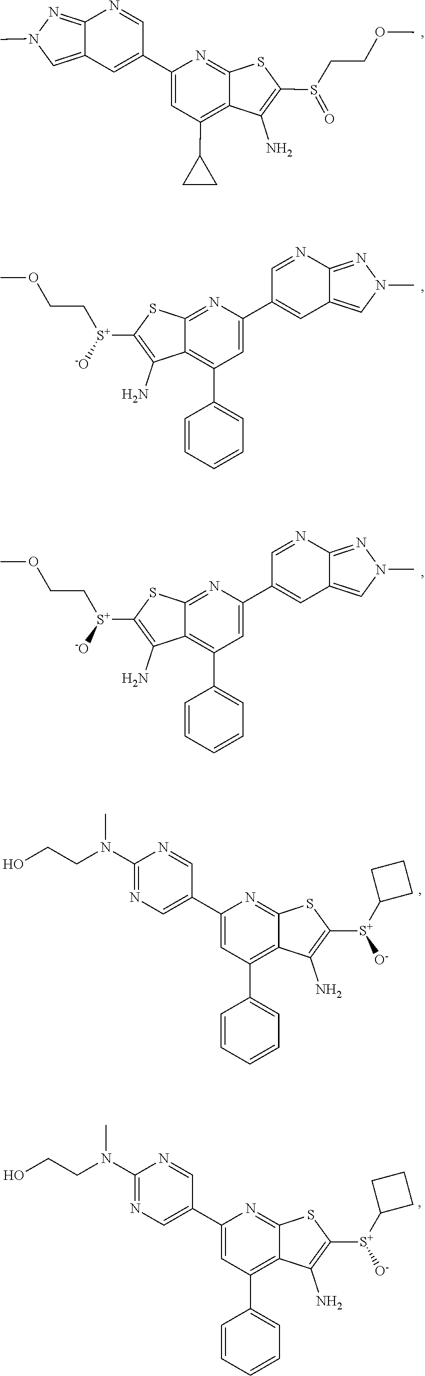

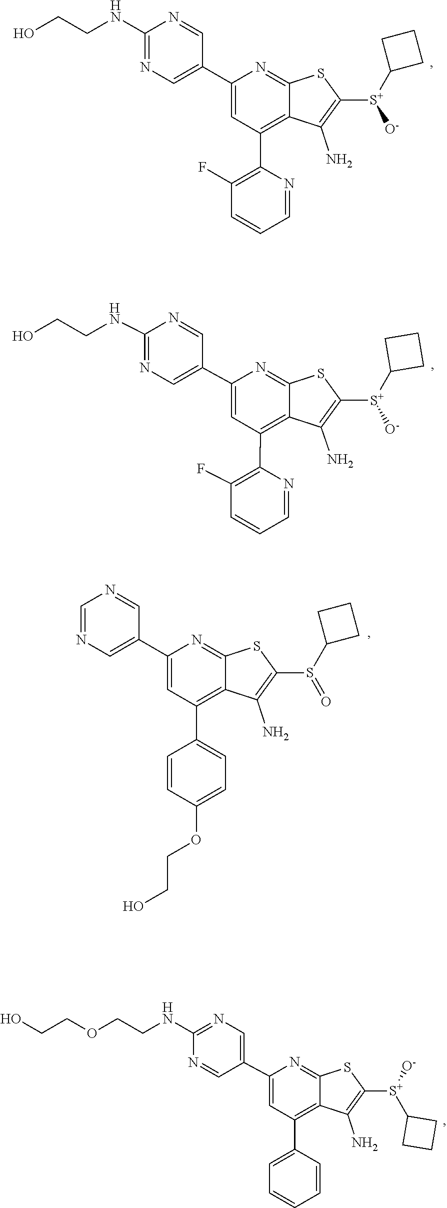

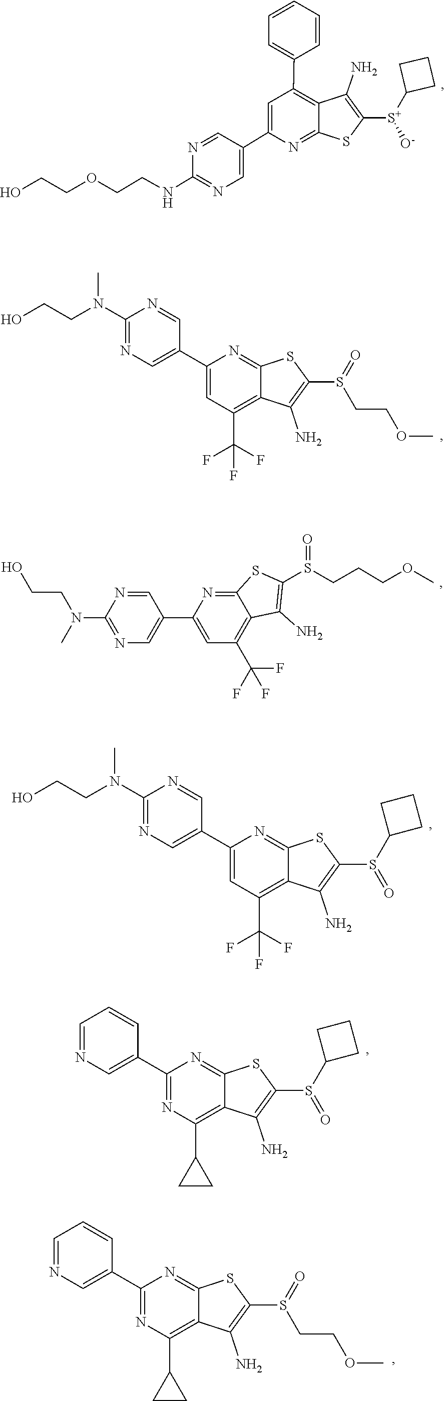

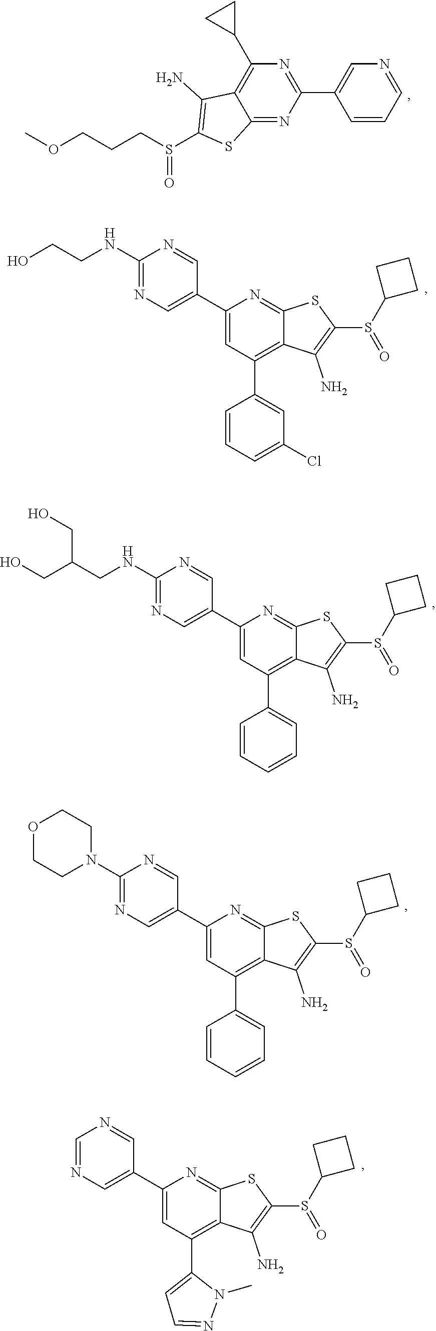 [SMILES: COCCCS(=O)c1sc2nc(-c3cccnc3)nc(C3CC3)c2c1N ; Nc1c(S(=O)C2CCC2)sc2nc(-c3cnc(NCCO)nc3)cc(-c3cccc(Cl)c3)c12 ; Nc1c(S(=O)C2CCC2)sc2nc(-c3cnc(NCC(CO)CO)nc3)cc(-c3ccccc3)c12 ; Nc1c(S(=O)C2CCC2)sc2nc(-c3cnc(N4CCOCC4)nc3)cc(-c3ccccc3)c12 ; Cn1nccc1-c1cc(-c2cncnc2)nc2sc(S(=O)C3CCC3)c(N)c12]

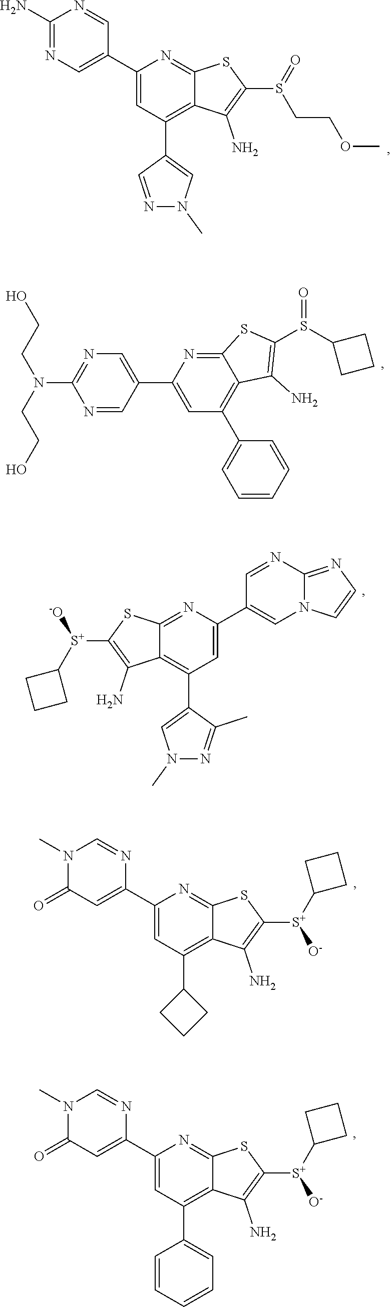

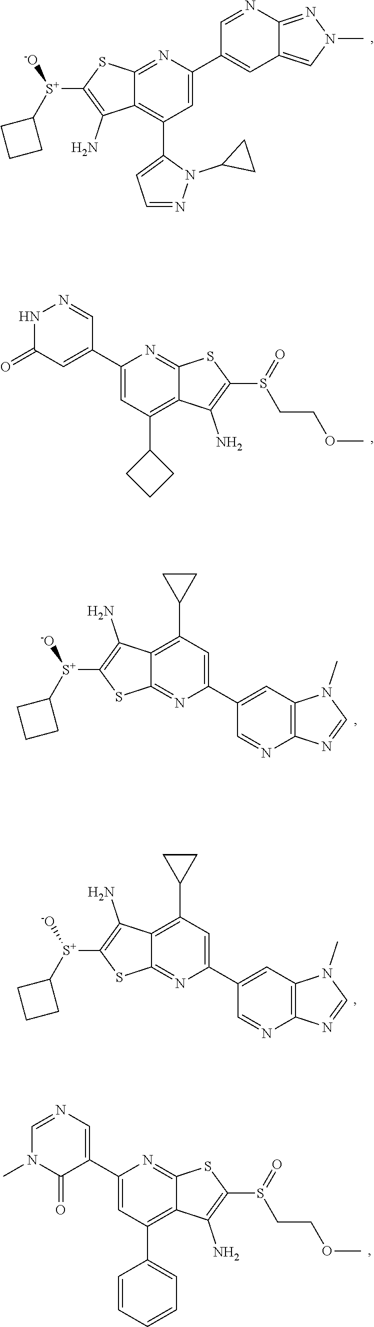

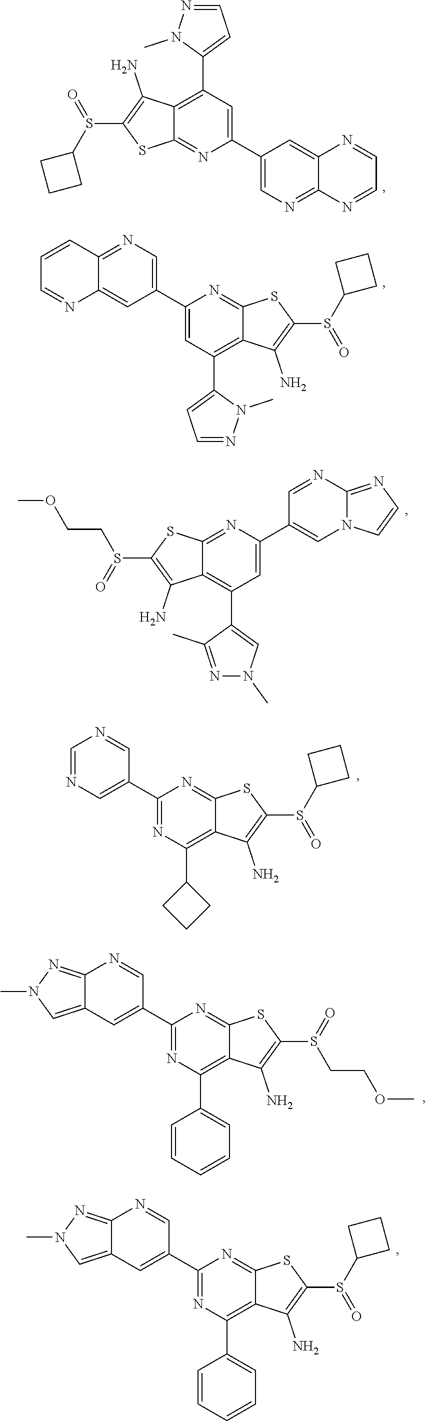

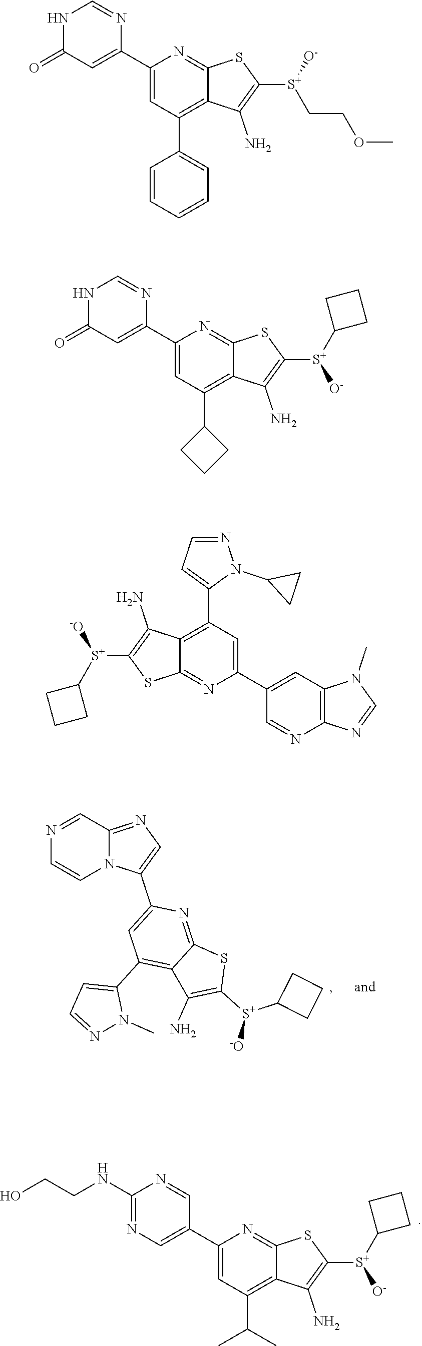

or pharmaceutically acceptable salts thereof, a tautomer thereof, and a solvates thereof.

In other embodiments, the compound can include at least one of the formulas (IB) or (IIB), or a pharmaceutically acceptable salt thereof:

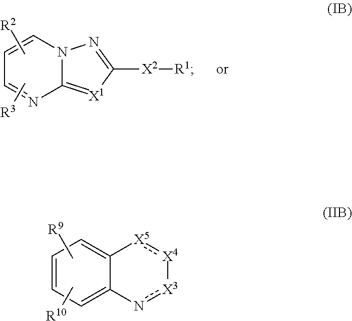

wherein X¹ is N or CR⁴;
X² is S, S═O, S(═O)₂, or C═O;
X³ is CR^{R}, the compound forming a polycyclic heteroaryl with 10 ring atoms, or absent, the compound forming a polycyclic heteroaryl with 9 ring atoms;
X⁴ is N, NH, or CR⁷;
X⁵ is N, C═O, or CR¹⁶, and X⁵ is N if X⁴ is CR⁷, or X³ is absent, X⁴ is NH if X⁵ is C═O, and X⁵ is CR¹⁶ if X⁴ is N and X³ is CR⁸;
R¹, R², R³, R⁴, R⁹, R¹⁰, and R¹⁶ are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-7 ring atoms, (wherein from 1-3 of the ring atoms is independently selected from N, NH, N(C₁-C₆ alkyl), NC(O) (C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano (—CN), isocyano (—N⁺C⁻), cyanato (—O—CN), isocyanato (—O—N⁺═C—), isothiocyanato (—S—CN), azido (—N═N⁺═N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, aryl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl], groups incorporating amino acids or other moieties expected to bear positive or negative charge at physiological pH, and combinations thereof;
R⁷ and R⁸ are same or different and are each independently selected from the group consisting of H, a substituted or unsubstituted aryl, a substituted or unsubstituted heteroaryl, a substituted or unsubstituted cycloalkyl, and a substituted or unsubstituted heterocyclyl, and at least one of R⁷ or R⁸ is not H; and
wherein the compound is not

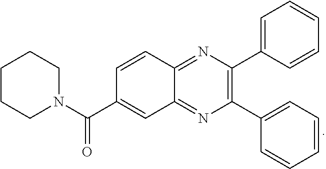 [SMILES: O=C(c1ccc2nc(-c3ccccc3)c(-c3ccccc3)nc2c1)N1CCCCC1]

In some embodiments, at least one of R² or R³ is not H, and at least one of R⁹ or R¹⁰ is not H.

In some embodiments, the 15-PGDH inhibitor can include a compound having at least one of the following formulas:

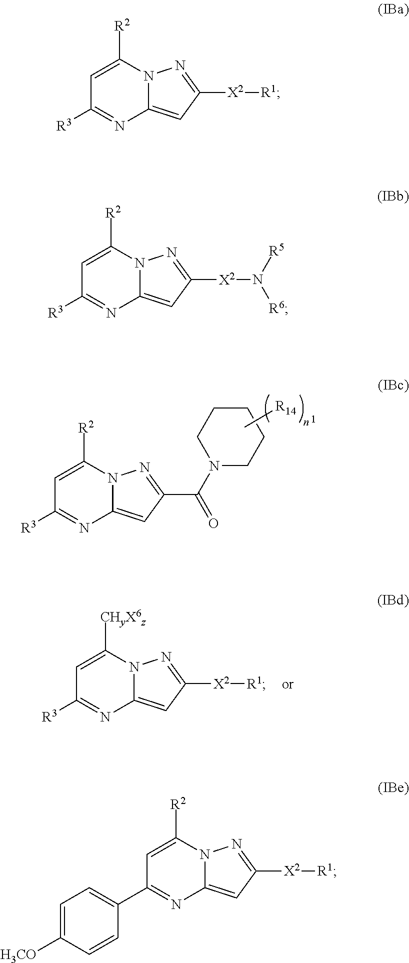

or a pharmaceutically acceptable salt thereof;
wherein X² is S, S═O, S(═O)₂, or C═O;
X⁶ is C₁, Br, or F, and y+z=3;
R¹, R², R³, R⁵, R⁶, and R¹⁴ are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-7 ring atoms, (wherein from 1-3 of the ring atoms is independently selected from N, NH, N(C₁-C₆ alkyl), NC(O) (C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano (—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, aiyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—|(CH₂)ₙO|ₘ), phosphates, phosphate esters [—OP(O)(OR)₂ where R═H, methyl or other alkyl], groups incorporating amino acids or other moieties expected to bear positive or negative charge at physiological pH, and combinations thereof; wherein R⁵ and R⁶ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl, n¹ is 0-4, and each R¹⁴ is the same or different.

In other embodiments, the 15-PGDH inhibitor can include a compound having at least one of the following formulas:

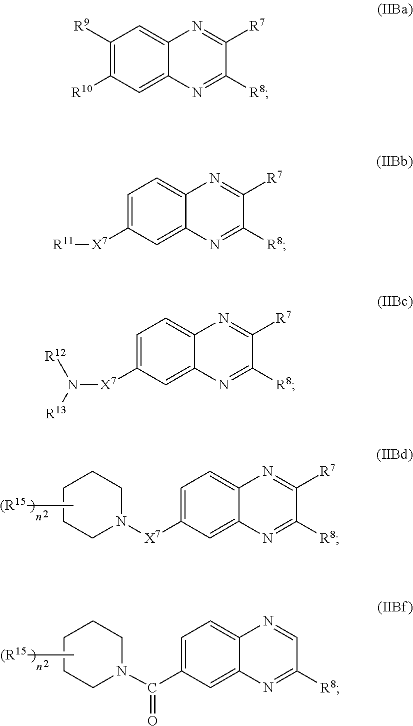

or a pharmaceutically acceptable salt thereof;
wherein X⁷ is S, S═O, S(═O)₂, or C═O;
R⁷ and R⁸ are same or different and are each independently selected from the group consisting of H, a substituted or unsubstituted aryl, a substituted or unsubstituted heteroaryl, a substituted or unsubstituted cycloalkyl, and a substituted or unsubstituted heterocyclyl, and at least one of R⁷ or R⁸ is not H;
R⁹, R¹⁰, R¹¹, R¹², R¹³, and R¹⁵ are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-7 ring atoms, (wherein from 1-3 of the ring atoms is independently selected from N, NH, N(C₁-C₆ alkyl), NC(O) (C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano (—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, aryl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R═H, methyl or other alkyl|, groups incorporating amino acids or other moieties expected to bear positive or negative charge at physiological pH, and combinations thereof; wherein R¹² and R¹³ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl, n² is 0-4, and each R¹⁵ is the same or different.

Examples of 15-PGDH inhibitors having formulas (IB), (IBa), (IBb), (IBc), (IBd), (IBe), (IIB), (IIBa), (IIBb), (IIBc), (IIBd), (IBe), or (IIBf) can include the following compounds:

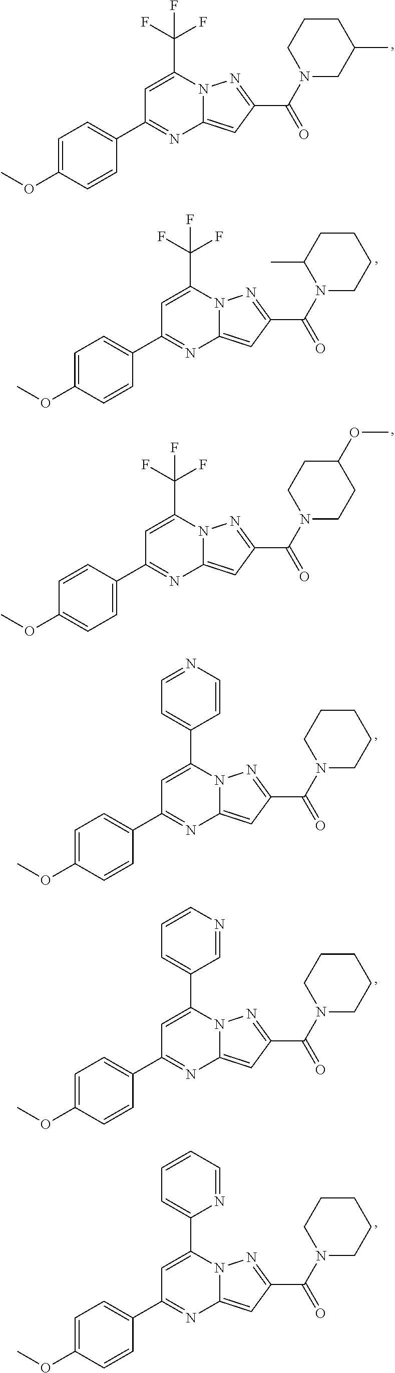

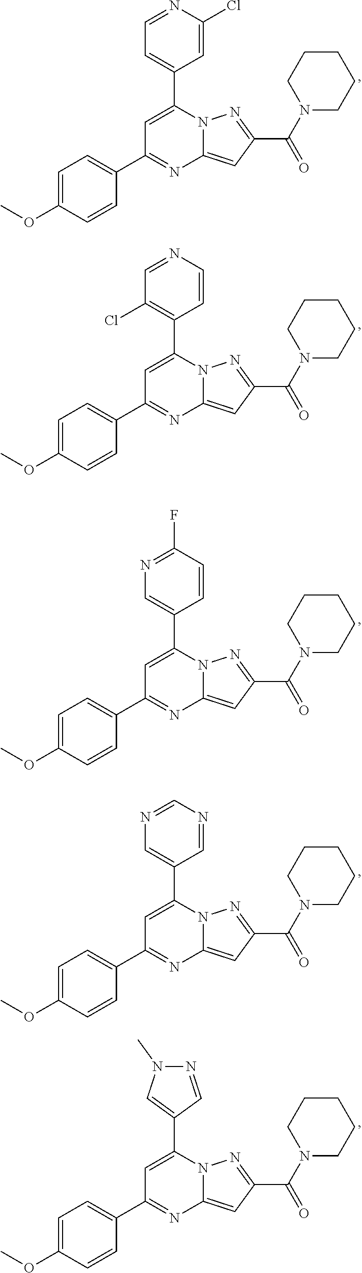

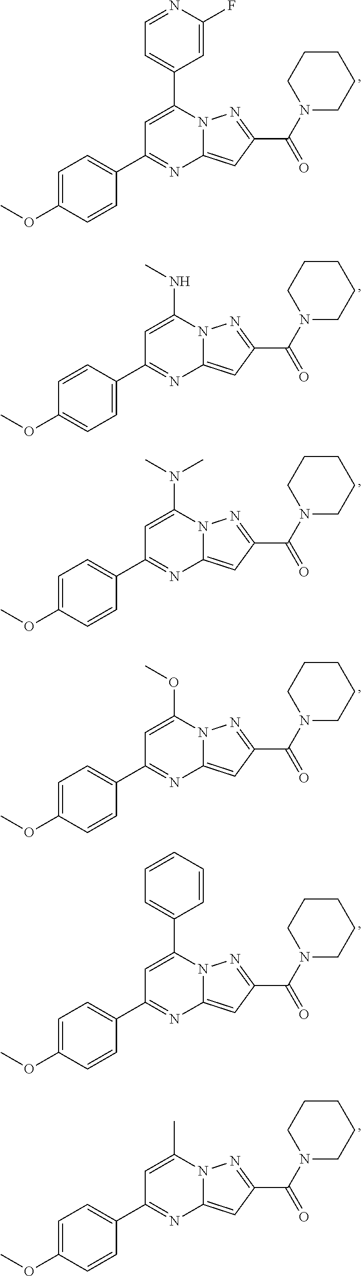 [SMILES: COc1ccc(-c2cc(-c3ccnc(F)c3)n3nc(C(=O)N4CCCCC4)cc3n2)cc1 ; CNc1cc(-c2ccc(OC)cc2)nc2cc(C(=O)N3CCCCC3)nn12 ; COc1ccc(-c2cc(N(C)C)n3nc(C(=O)N4CCCCC4)cc3n2)cc1 ; COc1ccc(-c2cc(OC)n3nc(C(=O)N4CCCCC4)cc3n2)cc1 ; COc1ccc(-c2cc(-c3ccccc3)n3nc(C(=O)N4CCCCC4)cc3n2)cc1 ; COc1ccc(-c2cc(C)n3nc(C(=O)N4CCCCC4)cc3n2)cc1]

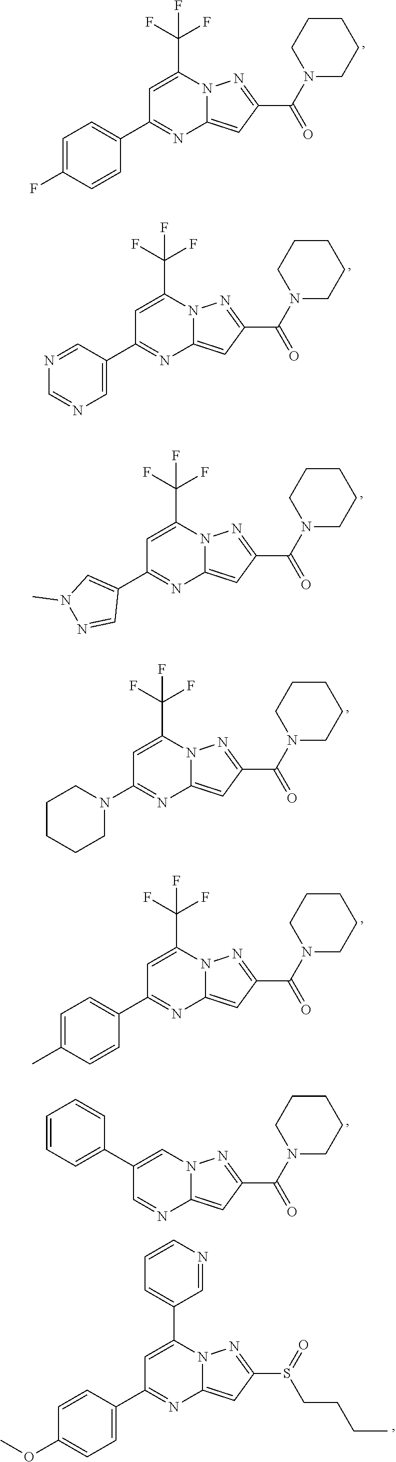

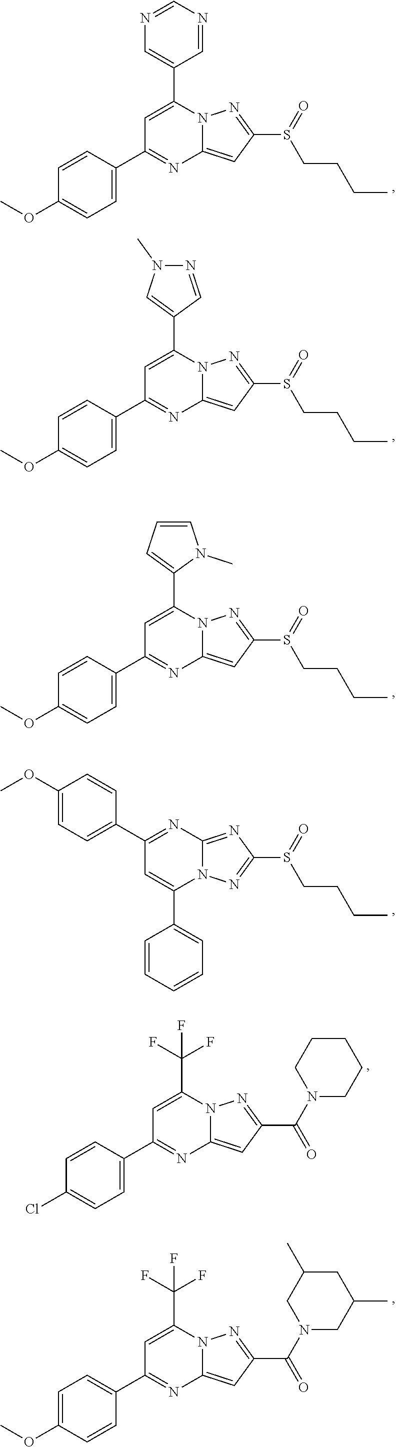

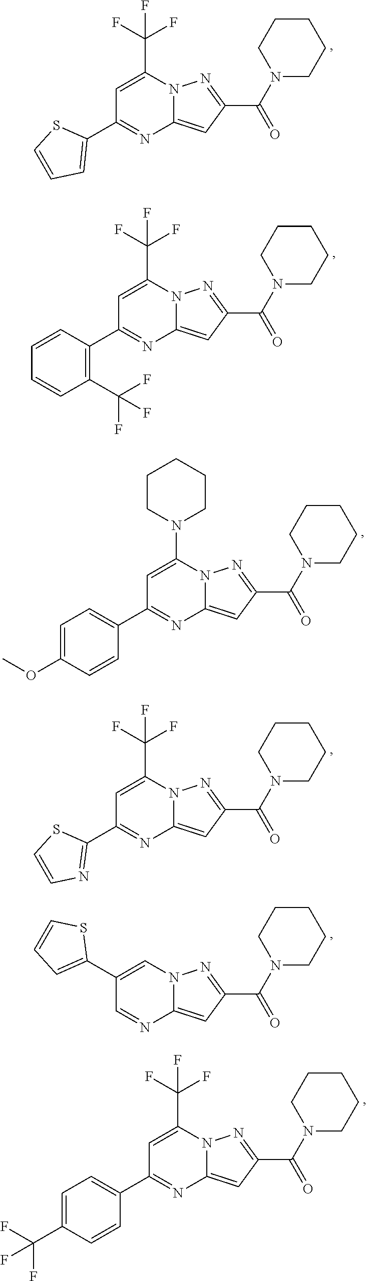

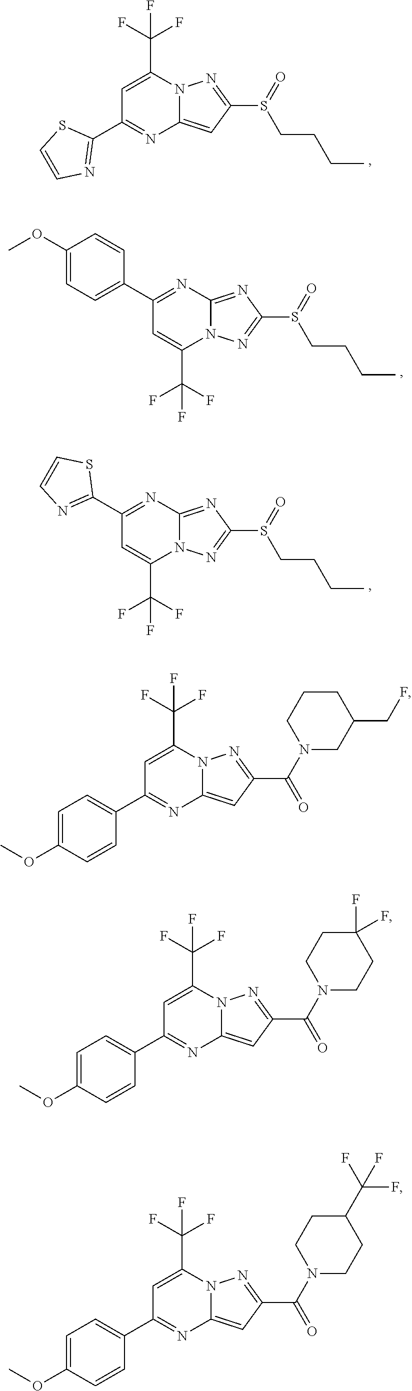

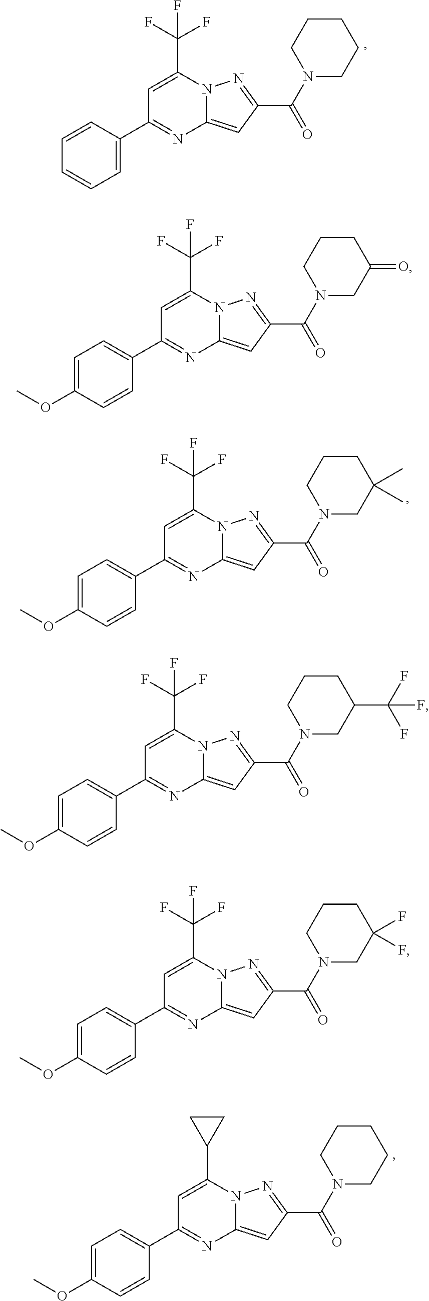

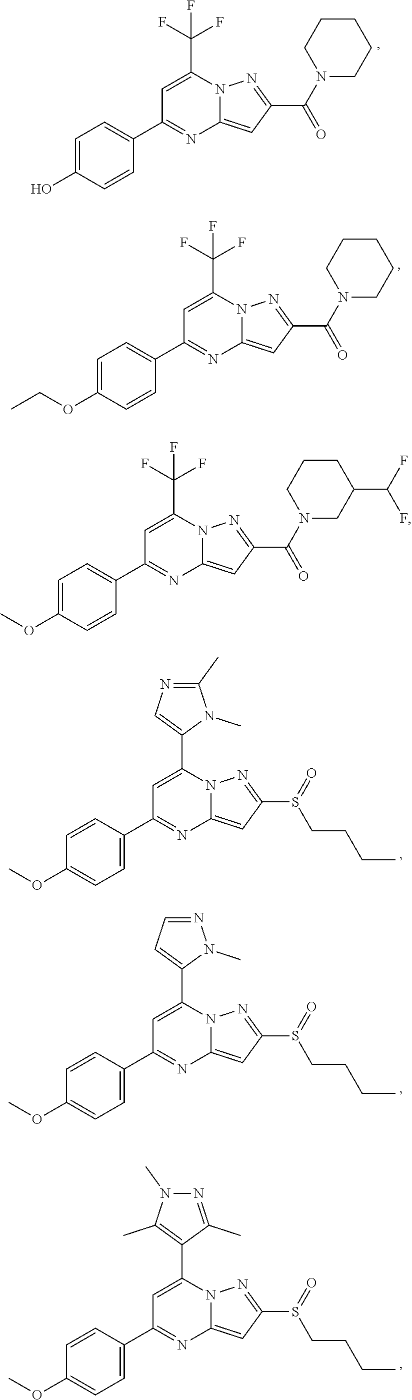

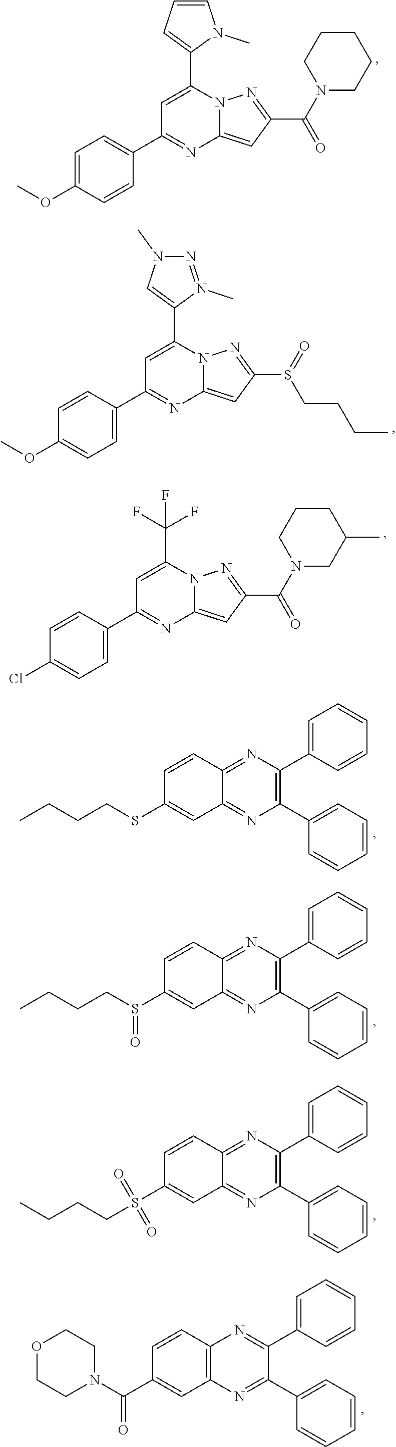

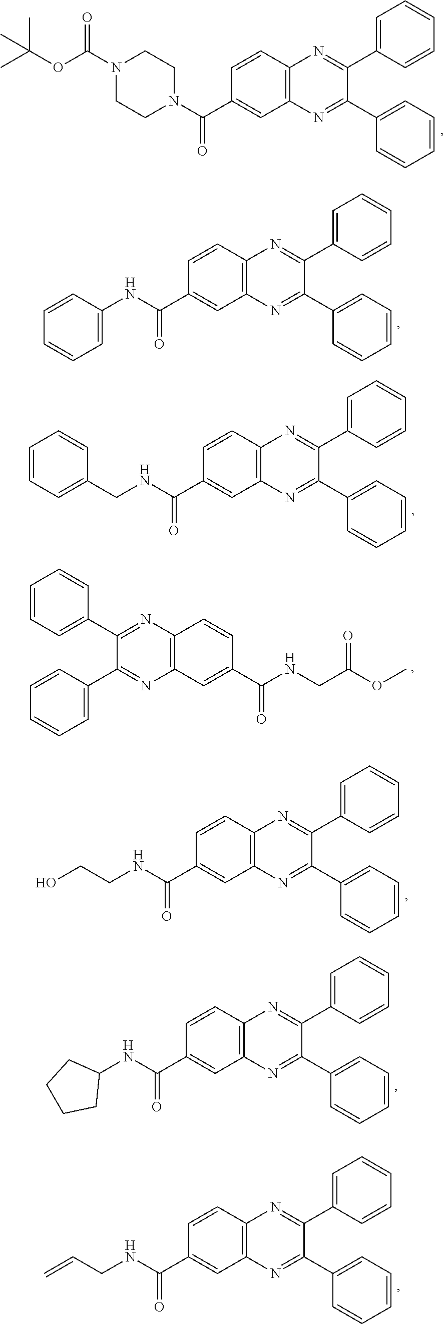

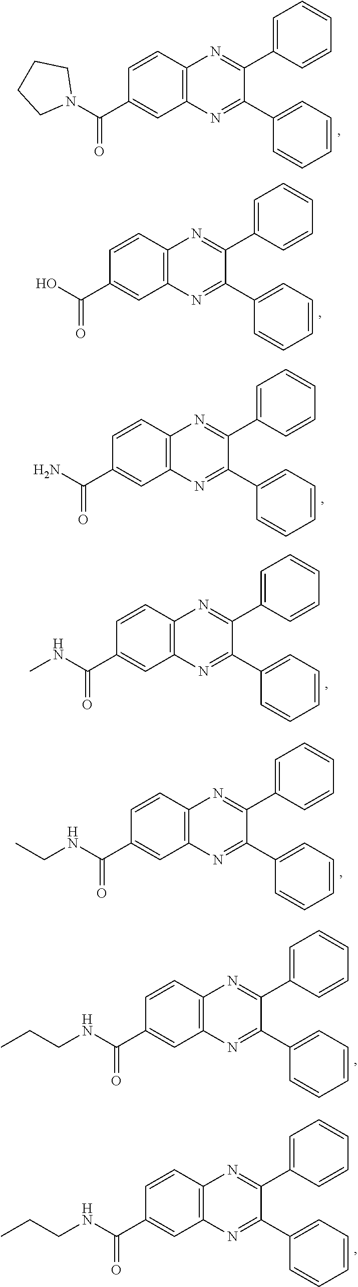

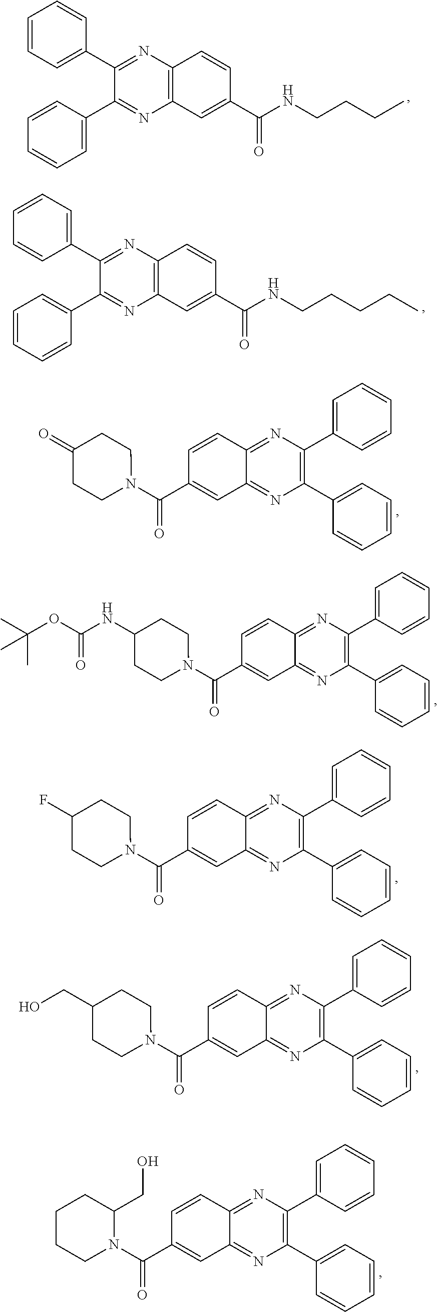

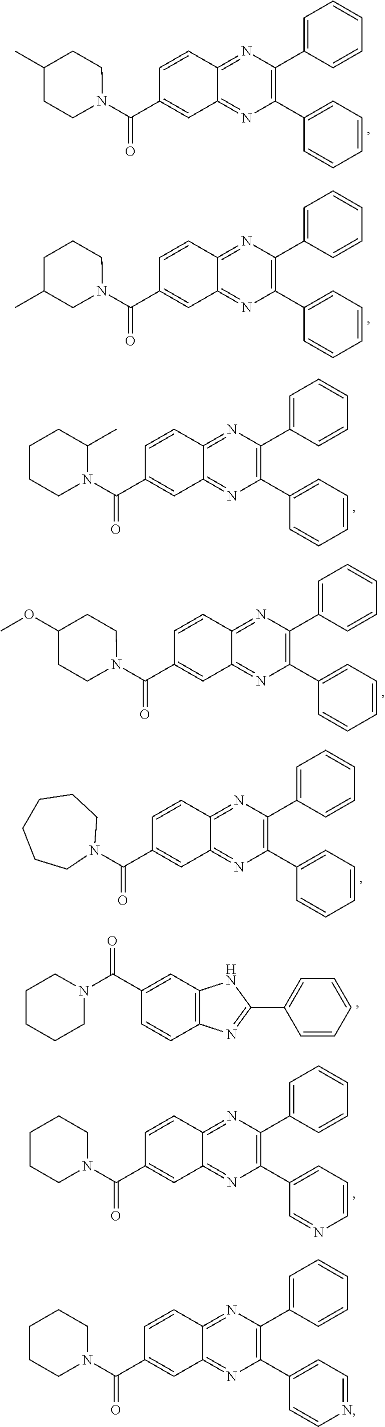

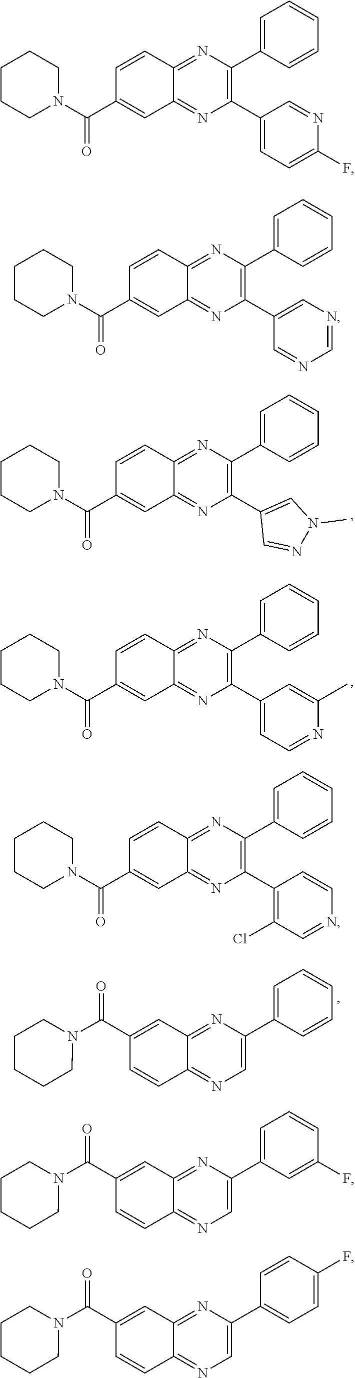

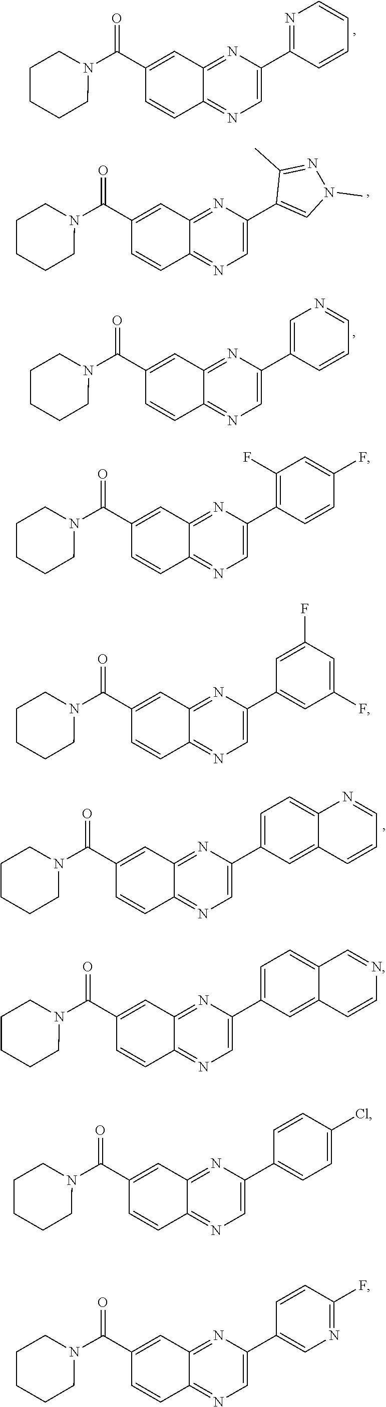

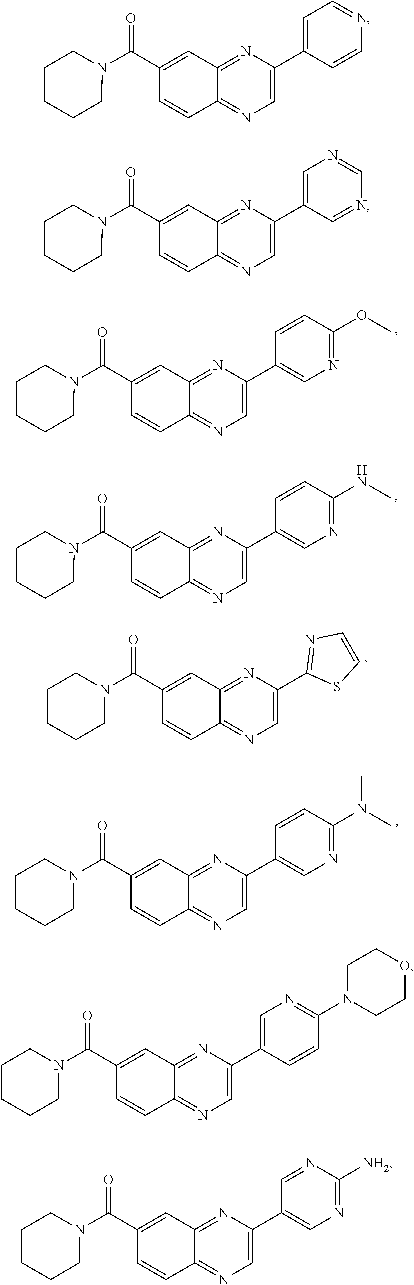

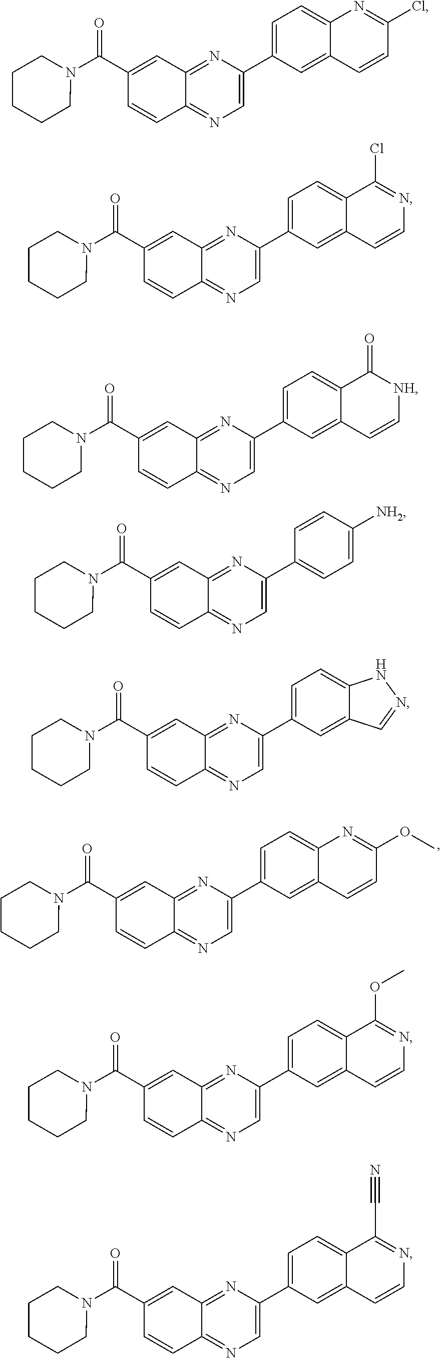

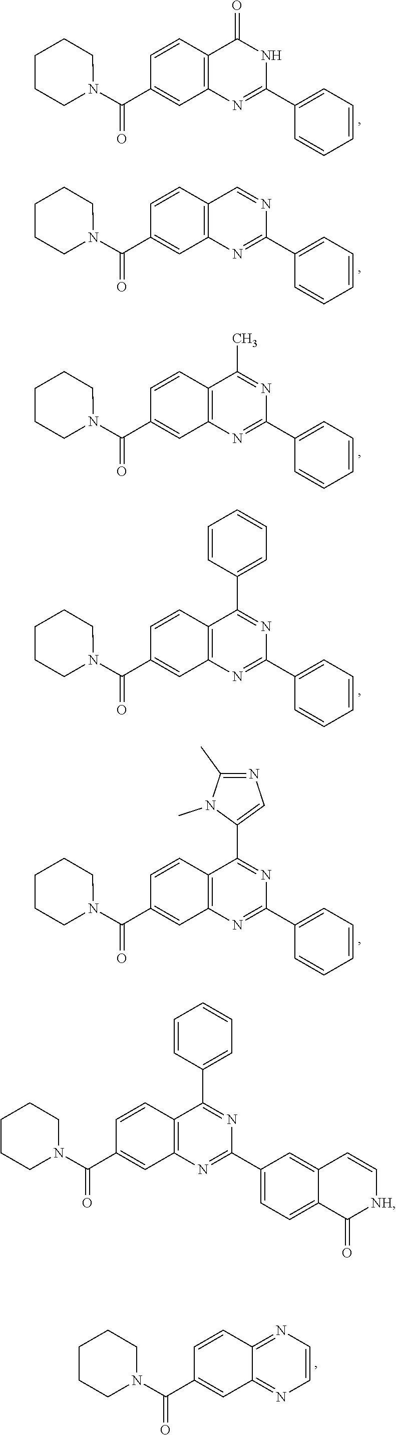

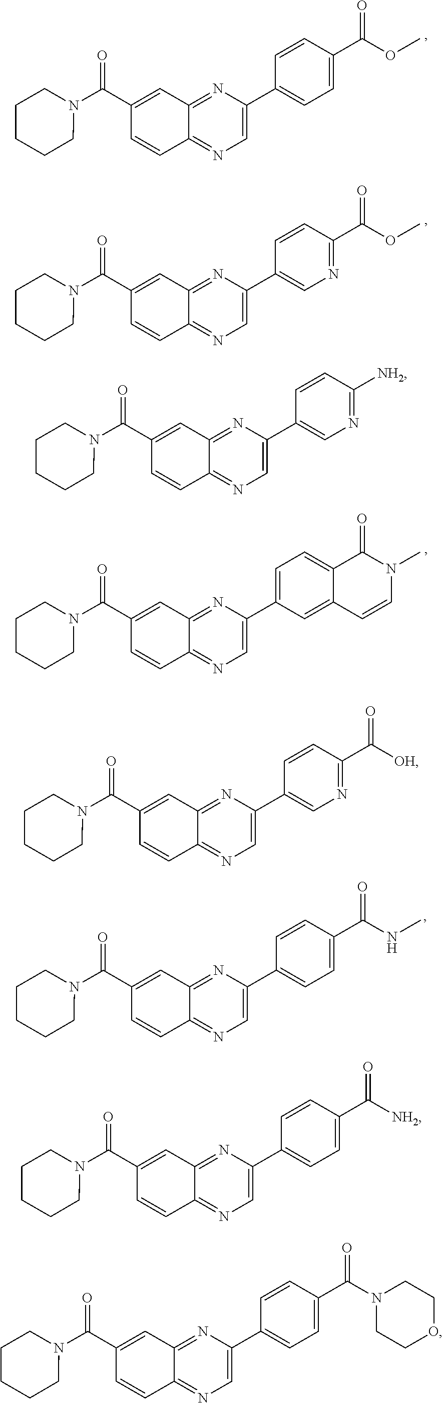

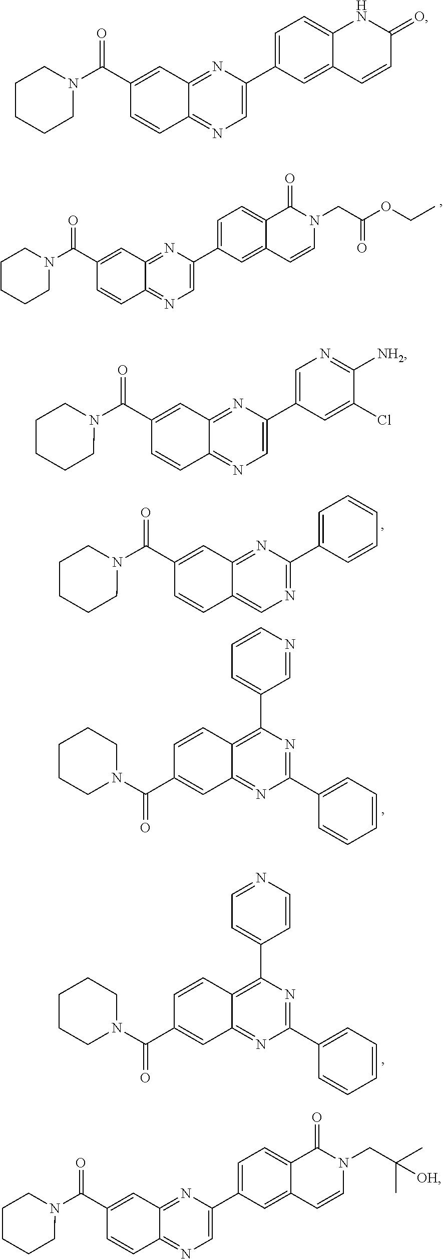

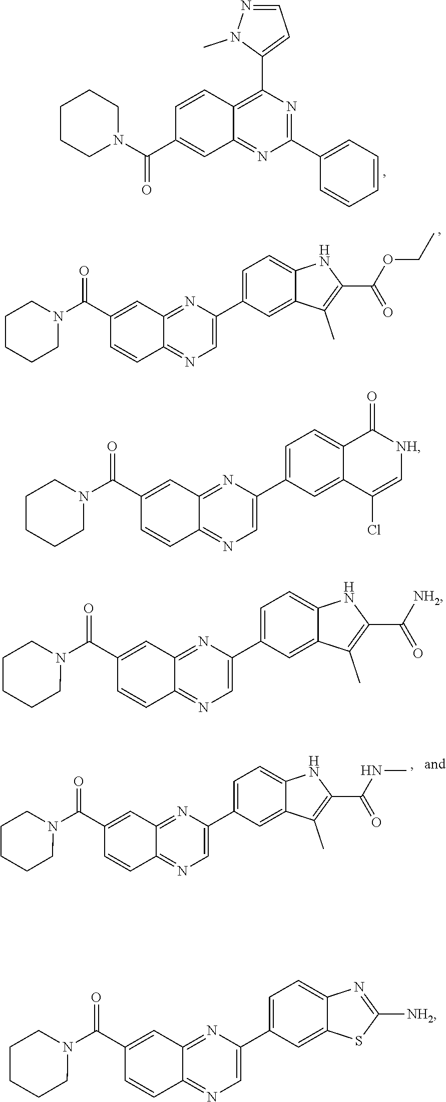 [SMILES: Cn1nccc1-c1nc(-c2ccccc2)nc2cc(C(=O)N3CCCCC3)ccc12 ; CCOC(=O)c1[nH]c2ccc(-c3cnc4ccc(C(=O)N5CCCCC5)cc4n3)cc2c1C ; O=C(c1ccc2ncc(-c3ccc4c(=O)[nH]cc(Cl)c4c3)nc2c1)N1CCCCC1 ; Cc1c(C(N)=O)[nH]c2ccc(-c3cnc4ccc(C(=O)N5CCCCC5)cc4n3)cc12 ; CNC(=O)c1[nH]c2ccc(-c3cnc4ccc(C(=O)N5CCCCC5)cc4n3)cc2c1C ; Nc1nc2ccc(-c3cnc4ccc(C(=O)N5CCCCC5)cc4n3)cc2s1]

or pharmaceutically acceptable salts thereof, a tautomer thereof, and a solvates thereof.

Still other examples of 15-PGDH inhibitors include compounds described in WO2022/032230A1, WO2021/236779A1, WO2020/106998A1, WO2018/218251A1, WO2018/145080A1, WO2016/168472A1, WO2015/065716A1, and WO2013/158649A1 all of which are herein incorporated by reference in its entirety.

In certain embodiments, the 15-PGDH inhibitor can be selected that can ia) at 2.5 μM concentration, stimulate a Vaco503 reporter cell line expressing a 15-PGDH luciferase fusion construct to a luciferase output level of greater than 70 (using a scale on which a value of 100 indicates a doubling of reporter output over baseline); iia) at 2.5 M concentration stimulate a V9m reporter cell line expressing a 15-PGDH luciferase fusion construct to a luciferase output level of greater than 75; iiia) at 7.5 μM concentration stimulate a LS174T reporter cell line expressing a 15-PGDH luciferase fusion construct to a luciferase output level of greater than 70; and iva) at 7.5 μM concentration, does not activate a negative control V9m cell line expressing TK-renilla luciferase reporter to a level greater than 20; and va) inhibits the enzymatic activity of recombinant 15-PGDH protein at an IC₅₀ of less than 1 μM.

In other embodiments, the 15-PGDH inhibitor can ib) at 2.5 μM concentration, stimulate a Vaco503 reporter cell line expressing a 15-PGDH luciferase fusion construct to increase luciferase output; iib) at 2.5 μM concentration stimulate a V9m reporter cell line expressing a 15-PGDH luciferase fusion construct to increase luciferase output; iiib) at 7.5 μM concentration stimulate a LS174T reporter cell line expressing a 15-PGDH luciferase fusion construct to increase luciferase output; ivb) at 7.5 μM concentration, does not activate a negative control V9m cell line expressing TK-renilla luciferase reporter to a luciferase level greater than 20% above background; and vb) inhibits the enzymatic activity of recombinant 15-PGDH protein at an IC₅₀ of less than 1 μM.

In other embodiments, the 15-PGDH inhibitor can inhibit the enzymatic activity of recombinant 15-PGDH at an IC₅₀ of less than 1 μM, or preferably at an IC₅₀ of less than 250 nM, or more preferably at an IC₅₀ of less than 50 nM, or more preferably at an IC₅₀ of less than 10 nM, or more preferably at an IC₅₀ of less than 5 nM at a recombinant 15-PGDH concentration of about 5 nM to about 10 nM.

It will be appreciated that other 15-PGDH inhibitors can be used in the methods described herein. These other 15-PGDH inhibitors can include known 15-PGDH inhibitors including, for example, tetrazole compounds of formulas (I) and (II), 2-alkylideneaminooxyacetamide compounds of formula (I), heterocyclic compounds of formulas (VI) and (VII), and pyrazole compounds of formula (III) described in U.S. Patent Application Publication No. 2006/0034786 and U.S. Pat. No. 7,705,041; benzylidene-1,3-thiazolidine compounds of formula (I) described in U.S. Patent Application Publication No. 2007/0071699; phenylfurylmethylthiazolidine-2,4-dione and phenylthienylmethylthiazolidine-2,4-dione compounds described in U.S. Patent Application Publication No. 2007/0078175; thiazolidenedione derivatives described in U.S. Patent Application Publication No. 2011/0269954; phenylfuran, phenylthiophene, or phenylpyrrazole compounds described in U.S. Pat. No. 7,294,641, 5-(3,5-disubstituted phenylazo)-2-hydroxybenzene-acetic acids and salts and lactones described in U.S. Pat. No. 4,725,676, and azo compounds described in U.S. Pat. No. 4,889,846.

Still other examples of 15-PGDH inhibitors are described in the following publications: Seo S Y et al. Effect of 15-hydroxyprostaglandin dehydrogenase inhibitor on wound healing. Prostaglandins Leukot Essent Fatty Acids. 2015; 97:35-41. doi: 10.1016/j.plefa.2015.03.005. PubMed PMID: 25899574; Piao Y L et al. Wound healing effects of new 15-hydroxyprostaglandin dehydrogenase inhibitors. Prostaglandins Leukot Essent Fatty Acids. 2014; 91(6):325-32. doi: 10.1016/j.plefa.2014.09.011. PubMed PMID: 25458900; Choi D et al. Control of the intracellular levels of prostaglandin E(2) through inhibition of the 15-hydroxyprostaglandin dehydrogenase for wound healing. Bioorg Med Chem. 2013; 21(15):4477-84. doi: 10.1016/j.bmc.2013.05.049. PubMed PMID: 23791868; Wu Y et al. Synthesis and biological evaluation of novel thiazolidinedione analogues as 15-hydroxyprostaglandin dehydrogenase inhibitors. J Med Chem. 2011; 54(14):5260-4. Epub 2011/06/10. doi: 10.1021/jm200390u. PubMed PMID: 21650226; Duveau D Y et al. Structure-activity relationship studies and biological characterization of human NAD(+)-dependent 15-hydroxyprostaglandin dehydrogenase inhibitors. Bioorg Med Chem Lett. 2014; 24(2):630-5. doi: 10.1016/j.bmcl.2013.11.081. PubMed PMID: 24360556; PMCID: PMC3970110; Duveau D Y et al. Discovery of two small molecule inhibitors, ML387 and ML388, of human NAD+-dependent 15-hydroxyprostaglandin dehydrogenase. Probe Reports from the NIH Molecular Libraries Program. Bethesda (MD)2010; Wu Y et al. Synthesis and SAR of thiazolidinedione derivatives as 15-PGDH inhibitors. Bioorg Med Chem. 2010; 18(4):1428-33. doi: 10.1016/j.bmc.2010.01.016. PubMed PMID: 20122835; Wu Y et al. Synthesis and biological evaluation of novel thiazolidinedione analogues as 15-hydroxyprostaglandin dehydrogenase inhibitors. J Med Chem. 2011; 54(14):5260-4. Epub 2011/06/10. doi: 10.1021/jm200390u. PubMed PMID: 21650226; Jadhav A et al. Potent and selective inhibitors of NAD+-dependent 15-hydroxyprostaglandin dehydrogenase (HPGD). Probe Reports from the NIH Molecular Libraries Program. Bethesda (MD)2010; Niesen F H et al. High-affinity inhibitors of human NAD-dependent 15-hydroxyprostaglandin dehydrogenase: mechanisms of inhibition and structure-activity relationships. PLoS One. 2010; 5(11):e13719. Epub 2010/11/13. doi: 10.1371/journal.pone.0013719. PubMed PMID: 21072165; PMCID: 2970562; Michelet, J. et al. Composition comprising at least one 15-PGDH inhibitor. US20080206320 A1, 2008; and Rozot, R et al. Care/makeup compositions comprising a 2-alkylideneaminooxyacetamide compound for stimulating the growth of the hair or eyelashes and/or slowing loss thereof. U.S. Pat. No. 7,396,525 B2, 2008.

The 15-PGDH inhibitors described herein can be used to treat, prevent, or reduce the symptoms or severity of any neural injury, neurodegeneration, neurodegenerative and/or neuropsychiatric conditions, diseases, and/or disorders associated with aberrant or enhanced GFAP levels and/or astrogliosis and aberrant 15-PGDH activity. In some embodiments, a subject having the neural injury, neurodegeneration, neurodegenerative and/or neuropsychiatric conditions, diseases, and/or disorders associated with aberrant or enhanced GFAP levels and/or astrogliosis and aberrant 15-PGDH activity can have or be at risk of memory loss, cognitive decline, axonal degeneration, neuronal cell death, glia cell damage, and/or blood brain barrier permeability and the 15-PGDH inhibitor can be administered to the subject at an amount effective to decrease GFAP levels and treat or prevent the memory loss, cognitive decline, axonal degeneration, neuronal cell death, glia cell damage, and/or blood brain barrier permeability.

Subjects amenable to treatment by 15-PGDH inhibitors as disclosed herein include subjects having aberrant or enhanced GFAP levels and/or astrogliosis and at risk of a neurodegenerative condition, disease, or disorder but not showing symptoms (for example asymptomatic subjects), as well as subjects presently showing symptoms. In the case of dementia diseases, virtually anyone is at risk of suffering from dementia if he or she lives long enough. Therefore, the present methods can be administered prophylactically to the general population exhibiting aberrant or enhanced GFAP levels and/or astrogliosis without any assessment of the risk of the subject patient. The methods as disclosed herein are especially useful for individuals who do have a known genetic risk of neurodegenative condition, disease, of disorder and exhibiting aberrant or enhanced GFAP levels and/or astrogliosis. Such individuals include those having relatives who have experienced this disease, and those whose risk is determined by analysis of GFAP levels, as disclosed herein.

Subjects can be further screened for their likelihood of having or developing a neurodegenerative condition, disease, or disorder based on a number of biochemical and genetic markers in addition to aberrant or enhanced GFAP levels and/or astrogliosis. For example, one can also diagnose a subject with increased risk of developing Alzheimer's Disease using genetic markers for Alzheimer's Disease. Genetic abnormality in a few families has been traced to chromosome 21 (St. George-Hyslop et al., Science 235:885-890, 1987). One genetic marker is, for example mutations in the APP gene, particularly mutations at position 717 and positions 670 and 671 referred to as the Hardy and Swedish mutations respectively (see Hardy, TINS, supra). Other markers of risk are mutations in the presenilin genes, PS1 and PS2, and ApoE4, family history of Alzheimer's Disease, hypercholesterolemia or atherosclerosis. Subjects with APP, PS1 or PS2 mutations are highly likely to develop Alzheimer's disease. ApoE is a susceptibility gene, and subjects with the e4 isoform of ApoE (ApoE4 isoform) have an increased risk of developing Alzheimer's disease. Test for subjects with ApoE4 isoform are disclosed in U.S. Pat. No. 6,027,896, which is incorporated in its entirety herein by reference. Other genetic links have been associated with increased risk of Alzheimer's disease, for example variances in the neuronal sortilin-related receptor SORLI may have increased likelihood of developing late-onset Alzheimer's disease (Rogaeva at al, Nat. Genet. 2007 February; 39(2):168-77). Other potential Alzheimer disease susceptibility genes, include, for example ACE, CHRNB2, CST3, ESR1, GAPDH, IDE, MTHFR, NCSTN, PRNP, PSEN1, TF, TFAM and TNF and be used to identify subjects with increased risk of developing Alzheimer's disease (Bertram et al, Nat. Genet. 2007 January; 39(1):17-23), as well as variences in the alpha-T catenin (VR22) gene (Bertram et al, J Med. Genet. 2007 January; 44(1):e63) and Insulin-degrading enzyme (IDE) and Kim et al, J Biol. Chem. 2007; 282:7825-32).

Neurodegenative conditions, disease, or disorders associated with cognitive decline or memory loss can be diagnosed using standard practice and the progression can be monitored over an extended period of time. One such method includes at least one of the following; (i) a memory assessment, (ii) an extensive neuropyschological exam, (iii) an examination by a geriatric neurologist and (iv) MRI imaging of the brain. Disease progression can be documented by changes in these parameters over time. In some embodiments, changes in the parameters of at least one of these assessments can be used to assess the efficacy of 15-PGDH inhibitor in the subject over time.

Other methods to diagnose a patient at risk of or having a neurodegenerative condition, disease or disorder, such as vascular dementia or Alzheimer's Disease, includes measurement of GFAP levels of blood of the subject and 15-PGDH activity and/or expression in the neurotissue, such as brain tissue, wherein increased GFAP levels in blood of the subject and 15-PGDH activity and/or expression compared to a control (e.g., normal or healthy neurotissue) is indicative of the subject having or at increased risk of the neurodegenerative condition, disease, or disorder.

In some embodiments, where the subject has or is at risk of blood brain barrier breakdown, direct detection of BBB breakdown can be assessed using MRI and injection of contrasting agent. Improvements in the resolution of MRIs and in the use of special contrasting agents can be used to detect BBB permeability. In one method, subjects are administered a contrasting agent immediately prior to brain imaging, such as MRI imaging. In cases of intact BBB, the contrasting agents are confined to brain blood vessels whereas, in subjects with a disrupted BBB, the contrasting agent is “sprayed out” into the brain tissue, which can be visualized. Thus, the brain locations, the size of BBB breakdown and extent of BBB compromise, such as extent of vascular leak in subjects can be directly and quantitatively assessed as measurable parameters of BBB permeability. Furthermore, such methods can be used to assess any improvements in these parameters resulting from treatment of the subject with an agent that inhibits 15-PGDH activity. Direct visualization of BBB breakdown and its associated vascular leak into the brain is useful in the methods as disclosed herein for monitoring the beneficial effects of treating a subject with BBB permeability with a 15-PGDH inhibitor. An improvement in at least one measurable parameter of BBB permeability, such as location, size of BBB breakdown and extent of vascular leak in subjects administered a 15-PGDH inhibitor indicates a positive outcome from administration of an inhibitor of 15-PGDH. Parameters of BBB permeability can be monitored by direct visualization and quantified by MRI-associated image analysis and are useful in the methods as disclosed herein.

In some embodiments, the 15-PGDH inhibitors described herein are useful in preventing and treating neurodegenerative conditions, diseases and disorders, such as those associated with aberrant or enhanced GFAP levels and/or astrogliosis, aberrant 15-PGDH activity, and risk of memory loss, cognitive decline, axonal degeneration, neuronal cell death, glia cell damage, and/or blood brain barrier permeability. As discussed previously, such neurodegenerative conditions, diseases, or disorders can include subarachnoid hemorrhage, schizophrenia, depression, bipolar disorder, normal aging, epilepsy, traumatic brain injury and/or a visual symptom associated therewith, post-traumatic stress disorder, Parkinson's disease, Parkinson Plus syndromes, Lewy Body Dementia, multiple system atrophy, corticobasal neurodegeneration, progressive supranuclear palsy, Alexander's disease, Alzheimer's disease, Alzheimer's disease related dementias, Down syndrome, spinocerebellar ataxia, amyotrophic lateral sclerosis, Huntington's disease, stroke, brain radiation therapy, chronic stress, abuse or cellular toxicity of a neuro-active drug, retinal degeneration, spinal cord injury, peripheral nerve injury, idiopathic peripheral neuropathy, cognitive decline and/or general frailty associated with normal aging and/or chemotherapy, chemotherapy induced neuropathy, concussive injury, peripheral nerve crush injury, peripheral neuropathy, diabetic neuropathy, post-traumatic headache, multiple sclerosis, retinal degeneration and dystrophy, Leber congenital amaurosis, retinitis pigmentosa, cone-rod dystrophy, mnicrophthalhnia, anophthalmia, myopia, and hyperopia, spinal cord injury, traumatic spinal cord injury, peripheral nerve injury, retinal neuronal death related diseases, retinal trauma, Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy, Hereditary spastic paraparesis, Friedreich ataxia, Non-Friedreich ataxia, Spinocerebellar atrophies, Amyloidoses, Metabolic-related neurodegenerative disorders, Toxin-related neurodegenerative disorders, Multiple sclerosis, Charcot Marie Tooth, Diabetic neuropathy, Metabolic neuropathies, Endocrine neuropathies, Creutzfeldt-Jacob Disease, Primary progressive aphasia, Frontotemporal Lobar Degeneration, Cortical blindness, Shy-Drager Syndrome, Diffuse cerebral cortical atrophy of non-Alzheimer type, Lewy-body dementia, Pick disease, Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, Tourette syndrome, or viral infection.

Additional examples of neurodegenerative diseases or disorders associated with aberrant or enhanced GFAP levels and/or astrogliosis include, for example, polyglutamine repeat disorders such as Spinocerebellar ataxias (e.g., types 1, 2, 3, 6, 7 and 17), Machado-Joseph disease, Spinal and Bulbar muscular atrophy (SBMA or Kennedy's disease), Dentatorubral Pallidoluysian Atrophy (DRPLA) and other neurological conditions arising from polyglutamine expansions, or disease arising from non-coding DNA repeat expansions such as Fragile X syndrome, Fragile XE mental retardation, Friedreich ataxia, myotonic dystrophy, Spinocerebellar ataxias (types 8, 10 and 12) or other neurodegenerative diseases such as spinal muscular atrophy (Werdnig-Hoffman disease, Kugelberg-Welander disease), and spongiform encephalopathies.

Additional neurodegenerative diseases associated with aberrant or enhanced GFAP levels and/or astrogliosis for which agents inhibiting 15-PGDH can be useful include, for example, age-related memory impairment, agyrophilic grain dementia, Parkinsonism-dementia complex of Guam, auto-immune conditions (eg Guillain-Barre syndrome, Lupus), Biswanger's disease, brain and spinal tumors (including neurofibromatosis), cerebral amyloid angiopathies (Journal of Alzheimer's Disease vol 3, 65-73 (2001)), cerebral palsy, chronic fatigue syndrome, corticobasal degeneration, conditions due to developmental dysfunction of the CNS parenchyma, conditions due to developmental dysfunction of the cerebrovasculature, dementia—multi infarct, dementia—subcortical, dementia with Lewy bodies, dementia of human immunodeficiency virus (HIV), dementia lacking distinct histology, Dementia Pugilistica, diffies neurofibrillary tangles with calcification, diseases of the eye, ear and vestibular systems involving neurodegeneration (including macular degeneration and glaucoma), dyskinesias (Paroxysmal), dystonias, essential tremor, Fahr's syndrome, fronto-temporal dementia and Parkinsonism linked to chromosome 17 (FTDP-17), frontotemporal lobar degeneration, frontal lobe dementia, hepatic encephalopathy, hereditary spastic paraplegia, hydrocephalus, pseudotumor cerebri and other conditions involving CSF dysffunction, Gaucher's disease, Hallervorden-Spatz disease, Korsakoffs syndrome, mild cognitive impairment, monomeric amyotrophy, motor neuron diseases, multiple system atrophy, multiple sclerosis and other demyelinating conditions (e.g., leukodystrophies), myalgic encephalomyelitis, myoclonus, neurodegeneration induced by chemicals, drugs and toxins, neurological manifestations of AIDS including AIDS dementia, neurological/cognitive manifestations and consequences of bacterial and/or virus infections, including but not restricted to enteroviruses, Niemann-Pick disease, non-Guamanian motor neuron disease with neurofibrillary tangles, non-ketotic hyperglycinemia, olivo-ponto cerebellar atrophy, oculopharyugeal muscular dystrophy, neurological manifestations of Polio myelitis including non-paralytic polio and post-polio-syndrome, primary lateral sclerosis, prion diseases including Creutzfeldt-Jakob disease (including variant form), kuru, fatal familial insomnia, Gerstmann-Straussler-Scheinker disease and other transmissible spongiform encephalopathies, prion protein cerebral amyloid angiopathy, postencephalitic Parkinsonism, progressive muscular atrophy, progressive bulbar palsy, progressive subcortical gliosis, progressive supranuclear palsy, restless leg syndrome, Rett syndrome, Sandhoff disease, spasticity, sporadic fronto-temporal dementias, striatonigral degeneration, subacute sclerosing panencephalitis, sulphite oxidase deficiency, Sydenham's chorea, tangle only dementia, Tay-Sach's disease, Tourette's syndrome, vascular dementia, and Wilson disease.

Additional neurodegenerative diseases associated with aberrant or enhanced GFAP levels and/or astrogliosis for which 15-PGDH inhibitors are useful include other dementias not listed above, such as but without limitation, other mixed dementia, frontotemporal dementia, progressive supranuclear palsy (PSP), Parkinson's Disease with associated dementia, corticobasal degeneration, multiple system atrophy, HIV-induced dementia, white matter disease-associated dementias, mild cognitive impairment (MCI).

In some embodiments, the 15-PGDH inhibitors described herein are useful in preventing and treating blood brain barrier (BBB) permeability in a subject. Additional examples of diseases and disorders where BBB permeability occurs include, for example, multiple sclerosis, cerebral amyloid angiopathy, diabetic retinopathy, prion disorders, amyotrophic lateral sclerosis (ALS), Stiff-person Syndrome, Spinocerebellar Ataxias, Friedreich Ataxia, Ataxia Telangiectasia, Bulbospinal Atrophy (Kennedy Syndrome), Spinal Muscular Atrophy, Neuronal storage diseases (lipofuscinoses), Mitochondrial encephalomyopathies, Leukodystrophies, Neural sequelae of spinal shock/blunt trauma, Hypertensive Cerebrovascular disease, such as Lacunar Infarcts, Slit hemorrhages, Hypertensive encephalopathy. BBB permeability also occurs in brain tumors such as those with ‘sinusoidal’ (aka high nutrient) vascular supply.

In other embodiments, the 15-PGDH inhibitor can be administered to a subject with aberrant or enhanced GFAP levels and/or astrogliosis associated with or caused by administration of a drug or therapeutic agent to the subject. The drug or therapeutic agent can be one one that interacts with brain-located cellular receptors (membranous and cytosolic) or one that interacts with physiological pathways in the brain (action on a transporter), or a drug targeted to an organ other than the brain that interacts with brain-located cellular receptors or impacts/interferes physiological pathways of the brain. Alternatively, the drug is for use in treating a brain-related pathology; the brain-related pathology may include depression, anxiety, stress, panic disorder, pain, epilepsy, dementia, suicidal ideation, Alzheimer's disease, Parkinson's disease. The drug may include a selective-serotonin reuptake inhibitor (SSRI), a selective norepinephrine reuptake inhibitor (SNRI), a selective dopamine reuptake inhibitor (SDRI), serotonin receptor agonist/antagonists (interacting with the ‘5-HT’ receptor family), dopamine receptor agonist/antagonists (interacting with the ‘DT’ receptor family), adrenergic receptor agonist/antagonists (interacting with the ‘alpha’ and ‘beta’ receptor families, including beta-2-adrenergic receptor agonists), GABA receptor agonist/antagonists (interacting with the ‘GABA_{A}’ and ‘GABA_{B}’ receptor families), acetylcholine receptor agonist/antagonists (interacting with the ‘muscarinic’ and ‘nicotinic’ receptor families), glutamate receptor agonists/antagonists (e.g., NMDA receptor agonist/antagonists), opioid receptor agonist/antagonists (interacting with the ‘delta’, ‘kappa’, ‘mu’, ‘nociceptin’ receptor families), histamine receptor agonist/antagonists (the ‘H₃’ receptor); or a drug targeting an organ other than the brain that interacts with brain-located cellular receptors or indirectly disrupts brain physiological pathways. The drug can result in a change in homeostasis to neurotransmitter-related physiological pathways. The drug can be intended for use in or is used in treating a neuropsychiatric condition. The condition can be, for example, depression, anxiety, panic disorder, suicidal ideation or stress. The drug can be any drug tested for use in or used to treat a neuropsychiatric condition but is preferably a neurotransmitter reuptake inhibitor such as an SSRI, SNRI, or SDRI or a neurotransmitter receptor agonist or antagonist (includes partial agonists/anatagonists), especially a serotonin or dopamine receptor.

In some embodiments, the 15-PGDH inhibitors described herein can be provided in a pharmaceutical composition. A pharmaceutical composition containing the 15-PGDH inhibitors described herein as an active ingredient may be manufactured by mixing the derivative with a pharmaceutically acceptable carrier(s) or an excipient(s) or diluting the 15-PGDH inhibitors with a diluent in accordance with conventional methods. The pharmaceutical composition may further contain fillers, anti-cohesives, lubricants, wetting agents, flavoring agents, emulsifying agents, preservatives and the like. The pharmaceutical composition may be formulated into a suitable formulation in accordance with the methods known to those skilled in the art so that it can provide an immediate, controlled or sustained release of the 15-PGDH inhibitors after being administered into a mammal.

In some embodiments, the pharmaceutical composition may be formulated into a parenteral or oral dosage form. The solid dosage form for oral administration may be manufactured by adding excipient, if necessary, together with binder, disintegrants, lubricants, coloring agents, and/or flavoring agents, to the 15-PGDH inhibitors and shaping the resulting mixture into the form of tablets, sugar-coated pills, granules, powder or capsules. The additives that can be added in the composition may be ordinary ones in the art. For example, examples of the excipient include lactose, sucrose, sodium chloride, glucose, starch, calcium carbonate, kaolin, microcrystalline cellulose, silicate and the like. Exemplary binders include water, ethanol, propanol, sweet syrup, sucrose solution, starch solution, gelatin solution, carboxymethylcellulose, hydroxypropyl cellulose, hydroxypropyl starch, methylcellulose, ethylcellulose, shellac, calcium phosphonate and polypyrrolidone. Examples of the disintegrant include dry starch, sodium arginate, agar powder, sodium bicarbonate, calcium carbonate, sodium lauryl sulfate, stearic monoglyceride and lactose. Further, purified talc, stearates, sodium borate, and polyethylene glycol may be used as a lubricant; and sucrose, bitter orange peel, citric acid, tartaric acid, may be used as a flavoring agent. In some embodiments, the pharmaceutical composition can be made into aerosol formulations (e.g., they can be nebulized) to be administered via inhalation.

The 15-PGDH inhibitors described herein may be combined with flavoring agents, buffers, stabilizing agents, and the like and incorporated into oral liquid dosage forms such as solutions, syrups or elixirs in accordance with conventional methods. One example of the buffers may be sodium citrate. Examples of the stabilizing agents include tragacanth, acacia and gelatin.

In some embodiments, the 15-PGDH inhibitors described herein may be incorporated into an injection dosage form, for example, for a subcutaneous, intramuscular or intravenous route by adding thereto pH adjusters, buffers, stabilizing agents, relaxants, topical anesthetics. Examples of the pH adjusters and the buffers include sodium citrate, sodium acetate and sodium phosphate. Examples of the stabilizing agents include sodium pyrosulfite, EDTA, thioglycolic acid and thiolactic acid. The topical anesthetics may be procaine HCl, lidocaine HCl and the like. The relaxants may be sodium chloride, glucose and the like.

In other embodiments, the 15-PGDH inhibitors described herein may be incorporated into suppositories in accordance with conventional methods by adding thereto pharmaceutically acceptable carriers that are known in the art, for example, polyethylene glycol, lanolin, cacao butter or fatty acid triglycerides, if necessary, together with surfactants such as Tween.

The pharmaceutical composition may be formulated into various dosage forms as discussed above and then administered through various routes including an oral, inhalational, transdermal, subcutaneous, intravenous or intramuscular route. The dosage can be a pharmaceutically or therapeutically effective amount.

Therapeutically effective dosages of 15-PDGH inhibitor is one that reduces activity and/or expression of 15-PGDH and GFAP levels and generates the maximum protective effect in preventing a neurodegenerative disease or disorder, or reduces a symptom of a neurodegenerative disease or disorder.

In some embodiments, an optimum dosage of the 15-PGDH inhibitor is one generating the maximum beneficial effect on damaged tissue. An effective dosage causes at least a statistically or clinically significant attenuation of at least one marker, symptom, or histological evidence characteristic of neurodegenerative condition, disease, or disorder. Markers, symptoms and histological evidence characteristic of neurodegenerative condition, disease, or disorder include memory loss, confusion, disturbances in axonal transport, demyelination, induction of metalloproteinases (MMPs), activation of glial cells, infiltration of lymphocytes, edema and immunological reactions that lead to tissue damage and further vascular injury. Stabilization of symptoms or diminution of tissue damage, under conditions wherein control patients or animals experience a worsening of symptoms or tissue damage, is one indicator of efficacy of a suppressive treatment.

Therapeutically effective dosage amounts of the 15-PGDH inhibitor may be present in varying amounts in various embodiments. For example, in some embodiments, a therapeutically effective amount of the 15-PGDH inhibitor may be an amount ranging from about 10-1000 mg (e.g., about 20 mg-1,000 mg, 30 mg-1,000 mg, 40 mg-1,000 mg, 50 mg-1,000 mg, 60 mg-1,000 mg, 70 mg-1,000 mg, 80 mg-1,000 mg, 90 mg-1,000 mg, about 10-900 mg, 10-800 mg, 10-700 mg, 10-600 mg, 10-500 mg, 100-1000 mg, 100-900 mg, 100-800 mg, 100-700 mg, 100-600 mg, 100-500 mg, 100-400 mg, 100-300 mg, 200-1000 mg, 200-900 mg, 200-800 mg, 200-700 mg, 200-600 mg, 200-500 mg, 200-400 mg, 300-1000 mg, 300-900 mg, 300-800 mg, 300-700 mg, 300-600 mg, 300-500 mg, 400 mg-1,000 mg, 500 mg-1,000 mg, 100 mg-900 mg, 200 mg-800 mg, 300 mg-700 mg, 400 mg-700 mg, and 500 mg-600 mg). In some embodiments, the 15-PGDH inhibitor is present in an amount of or greater than about 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg. In some embodiments, the 15-PGDH inhibitor is present in an amount of or less than about 1000 mg, 950 mg, 900 mg, 850 mg, 800 mg, 750 mg, 700 mg, 650 mg, 600 mg, 550 mg, 500 mg, 450 mg, 400 mg, 350 mg, 300 mg, 250 mg, 200 mg, 150 mg, or 100 mg.

In other embodiments, a therapeutically effective dosage amount may be, for example, about 0.001 mg/kg weight to 500 mg/kg weight, e.g., from about 0.001 mg/kg weight to 400 mg/kg weight, from about 0.001 mg/kg weight to 300 mg/kg weight, from about 0.001 mg/kg weight to 200 mg/kg weight, from about 0.001 mg/kg weight to 100 mg/kg weight, from about 0.001 mg/kg weight to 90 mg/kg weight, from about 0.001 mg/kg weight to 80 mg/kg weight, from about 0.001 mg/kg weight to 70 mg/kg weight, from about 0.001 mg/kg weight to 60 mg/kg weight, from about 0.001 mg/kg weight to 50 mg/kg weight, from about 0.001 mg/kg weight to 40 mg/kg weight, from about 0.001 mg/kg weight to 30 mg/kg weight, from about 0.001 mg/kg weight to 25 mg/kg weight, from about 0.001 mg/kg weight to 20 mg/kg weight, from about 0.001 mg/kg weight to 15 mg/kg weight, from about 0.001 mg/kg weight to 10 mg/kg weight.

In still other embodiments, a therapeutically effective dosage amount may be, for example, about 0.0001 mg/kg weight to 0.1 mg/kg weight, e.g. from about 0.0001 mg/kg weight to 0.09 mg/kg weight, from about 0.0001 mg/kg weight to 0.08 mg/kg weight, from about 0.0001 mg/kg weight to 0.07 mg/kg weight, from about 0.0001 mg/kg weight to 0.06 mg/kg weight, from about 0.0001 mg/kg weight to 0.05 mg/kg weight, from about 0.0001 mg/kg weight to about 0.04 mg/kg weight, from about 0.0001 mg/kg weight to 0.03 mg/kg weight, from about 0.0001 mg/kg weight to 0.02 mg/kg weight, from about 0.0001 mg/kg weight to 0.019 mg/kg weight, from about 0.0001 mg/kg weight to 0.018 mg/kg weight, from about 0.0001 mg/kg weight to 0.017 mg/kg weight, from about 0.0001 mg/kg weight to 0.016 mg/kg weight, from about 0.0001 mg/kg weight to 0.015 mg/kg weight, from about 0.0001 mg/kg weight to 0.014 mg/kg weight, from about 0.0001 mg/kg weight to 0.013 mg/kg weight, from about 0.0001 mg/kg weight to 0.012 mg/kg weight, from about 0.0001 mg/kg weight to 0.011 mg/kg weight, from about 0.0001 mg/kg weight to 0.01 mg/kg weight, from about 0.0001 mg/kg weight to 0.009 mg/kg weight, from about 0.0001 mg/kg weight to 0.008 mg/kg weight, from about 0.0001 mg/kg weight to 0.007 mg/kg weight, from about 0.0001 mg/kg weight to 0.006 mg/kg weight, from about 0.0001 mg/kg weight to 0.005 mg/kg weight, from about 0.0001 mg/kg weight to 0.004 mg/kg weight, from about 0.0001 mg/kg weight to 0.003 mg/kg weight, from about 0.0001 mg/kg weight to 0.002 mg/kg weight. In some embodiments, the therapeutically effective dose may be 0.0001 mg/kg weight, 0.0002 mg/kg weight, 0.0003 mg/kg weight, 0.0004 mg/kg weight, 0.0005 mg/kg weight, 0.0006 mg/kg weight, 0.0007 mg/kg weight, 0.0008 mg/kg weight, 0.0009 mg/kg weight, 0.001 mg/kg weight, 0.002 mg/kg weight, 0.003 mg/kg weight, 0.004 mg/kg weight, 0.005 mg/kg weight, 0.006 mg/kg weight, 0.007 mg/kg weight, 0.008 mg/kg weight, 0.009 mg/kg weight, 0.01 mg/kg weight, 0.02 mg/kg weight, 0.03 mg/kg weight, 0.04 mg/kg weight, 0.05 mg/kg weight, 0.06 mg/kg weight, 0.07 mg/kg weight, 0.08 mg/kg weight, 0.09 mg/kg weight, or 0.1 mg/kg weight. The effective dose for a particular individual can be varied (e.g., increased or decreased) over time, depending on the needs of the individual.

In some embodiments, a therapeutically effective dosage may be a dosage of g/kg/day, 50 g/kg/day, 100 μg/kg/day, 250 g/kg/day, 500 g/kg/day, 1000 μg/kg/day or more. In various embodiments, the amount of the 15-PGDH inhibitor or pharmaceutical salt thereof is sufficient to provide a dosage to a patient of between 0.01 μg/kg and 10 g/kg; 0.1 μg/kg and 5 μg/kg; 0.1 μg/kg and 1000 μg/kg; 0.1 μg/kg and 900 μg/kg; 0.1 μg/kg and 900 μg/kg; 0.1 μg/kg and 800 μg/kg; 0.1 μg/kg and 700 μg/kg; 0.1 μg/kg and 600 μg/kg; 0.1 μg/kg and 500 μg/kg; or 0.1 μg/kg and 400 μg/kg.

Particular doses or amounts to be administered in accordance with the present invention may vary, for example, depending on the nature and/or extent of the desired outcome, on particulars of route and/or timing of administration, and/or on one or more characteristics (e.g., weight, age, personal history, genetic characteristic, lifestyle parameter, severity of cardiac defect and/or level of risk of cardiac defect, etc., or combinations thereof). Such doses or amounts can be determined by those of ordinary skill. In some embodiments, an appropriate dose or amount is determined in accordance with standard clinical techniques. For example, in some embodiments, an appropriate dose or amount is a dose or amount sufficient to reduce a disease severity index score by 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100% or more. For example, in some embodiments, an appropriate dose or amount is a dose or amount sufficient to reduce a disease severity index score by 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100%. Alternatively, or additionally, in some embodiments, an appropriate dose or amount is determined through use of one or more in vitro or in vivo assays to help identify desirable or optimal dosage ranges or amounts to be administered.

Various embodiments may include differing dosing regimen. In some embodiments, the 15-PGDH inhibitor can be administered via continuous infusion. In some embodiments, the continuous infusion is intravenous. In other embodiments, the continuous infusion is subcutaneous. Alternatively, or additionally, in some embodiments, the 15-PGDH inhibitor can be administered bimonthly, monthly, twice monthly, triweekly, biweekly, weekly, twice weekly, thrice weekly, daily, twice daily, or on another clinically desirable dosing schedule. The dosing regimen for a single subject need not be at a fixed interval, but can be varied over time, depending on the needs of the subject.

For topical application, the composition can be administered in the form of aqueous, alcoholic, aqueous-alcoholic or oily solutions or suspensions, or of a dispersion of the lotion or serum type, of emulsions that have a liquid or semi-liquid consistency or are pasty, obtained by dispersion of a fatty phase in an aqueous phase (O/W) or vice versa (W/O) or multiple emulsions, of a free or compacted powder to be used as it is or to be incorporated into a physiologically acceptable medium, or else of microcapsules or microparticles, or of vesicular dispersions of ionic and/or nonionic type. It may thus be in the form of a salve, a tincture, milks, a cream, an ointment, a powder, a patch, an impregnated pad, a solution, an emulsion or a vesicular dispersion, a lotion, aqueous or anhydrous gels, a spray, a suspension, a shampoo, an aerosol or a foam. It may be anhydrous or aqueous. It may also comprise solid preparations constituting soaps or cleansing cakes.

Pharmaceutical compositions including the 15-PGDH inhibitor described herein can additionally contain, for example, at least one compound chosen from prostaglandins, in particular prostaglandin PGE₁, PGE₂, their salts, their esters, their analogues and their derivatives, in particular those described in WO 98/33497, WO 95/11003, JP 97-100091, JP 96-134242, in particular agonists of the prostaglandin receptors. It may in particular contain at least one compound such as the agonists (in acid form or in the form of a precursor, in particular in ester form) of the prostaglandin F₂α receptor, such as for example latanoprost, fluprostenol, cloprostenol, bimatoprost, unoprostone, the agonists (and their precursors, in particular the esters such as travoprost) of the prostaglandin E₂ receptors such as 17-phenyl PGE₂, viprostol, butaprost, misoprostol, sulprostone, 16,16-dimethyl PGE₂, 11-deoxy PGE₁, 1-deoxy PGE₁, the agonists and their precursors, in particular esters, of the prostacycline (IP) receptor such as cicaprost, iloprost, isocarbacycline, beraprost, eprostenol, treprostinil, the agonists and their precursors, in particular the esters, of the prostaglandin D2 receptor such as BW245C ((4S)-(3-[(3R,S)-3-cyclohexyl-3-isopropyl]-2,5-dioxo)-4-imidazolidinehept-anoic acid), BW246C ((4R)-(3-[(3R,S)-3-cyclohexyl-3-isopropyl]-2,5-dioxo)-4-imidazolidinehept-anoic acid), the agonists and their precursors, in particular the esters, of the receptor for the thromboxanes A2 (TP) such as I—BOP ([1S-[1a,2a(Z), 3b(1E,3S),4a]]-7-[3-[3-hydroxy-4-[4-(iodophenoxy)-1-butenyl]-7-oxabicyclo-[2.2.1]hept-2-yl]-5-heptenoic acid).

Advantageously, the composition can include at least one 15-PGDH inhibitor as defined above and at least one prostaglandin or one prostaglandin derivative such as for example the prostaglandins of series 2 including in particular PGF2, and PGE₂ in saline form or in the form of precursors, in particular of the esters (example isopropyl esters), their derivatives such as 16,16-dimethyl PGE₂, 17-phenyl PGE₂ and 16,16-dimethyl PGF₂a 17-phenyl PGF2α, prostaglandins of series 1 such as 11-deoxyprostaglandin E1, 1-deoxyprostaglandin E1 in saline or ester form, is their analogues, in particular latanoprost, travoprost, fluprostenol, unoprostone, bimatoprost, cloprostenol, viprostol, butaprost, misoprostol, their salts or their esters.

The invention is further illustrated by the following examples, which is not intended to limit the scope of the claims.

### EXAMPLES

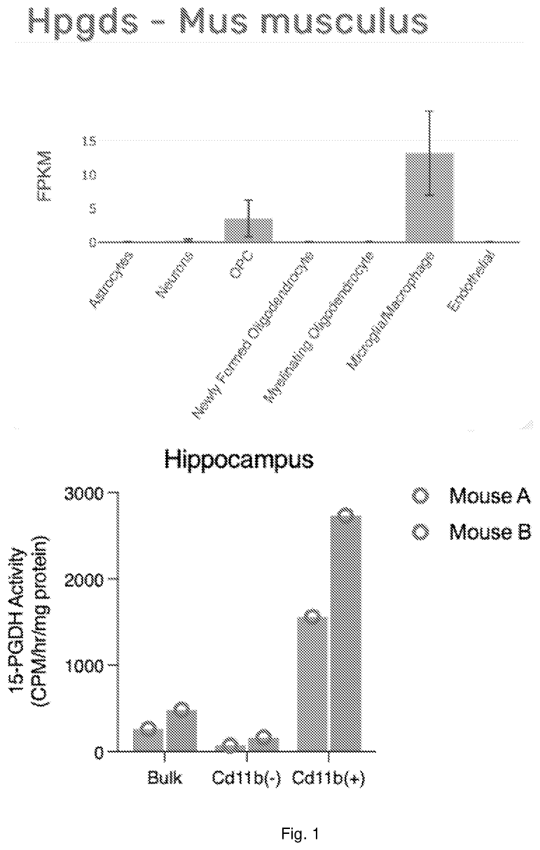

FIG. 1 shows that 15-PGDH activity is highly enriched in CD11b+ cells (microglial/macrophages) in the brain.

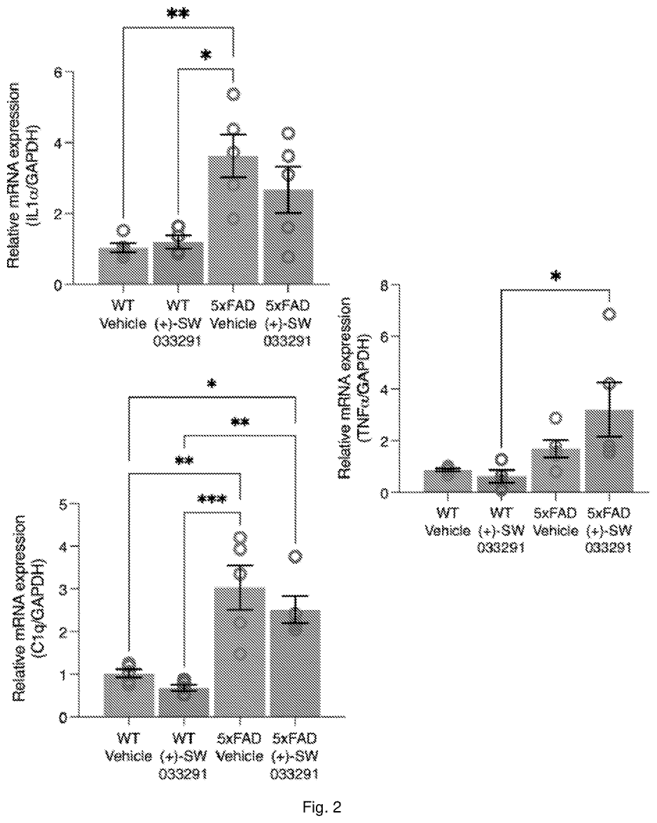

FIG. 2 shows inhibition of 15-PGDH does not suppress pro-inflammatory cytokine production in 5×FAD mice.

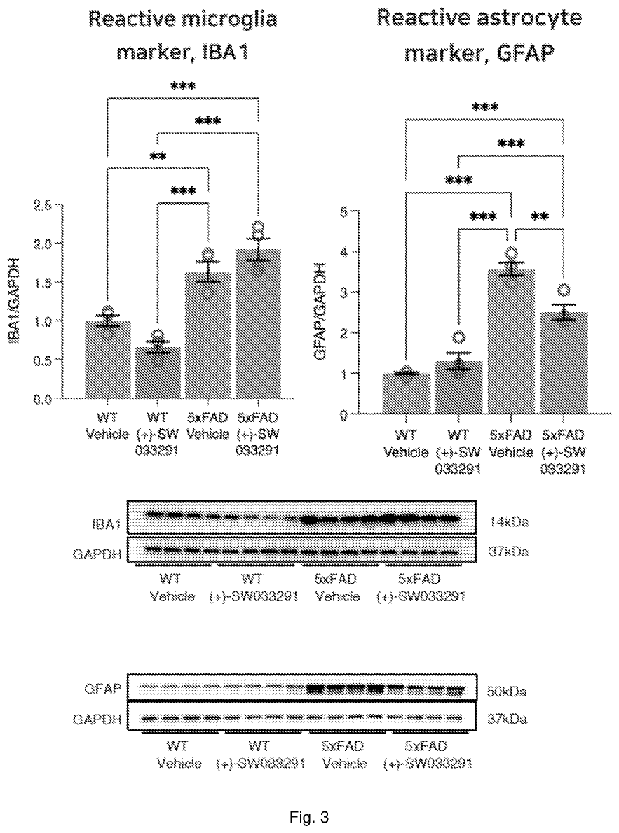

FIG. 3 shows that inhibition of 15-PGDH suppresses reactive astrocyte marker, GFAP expression in 5×FAD mice and that such suppression of GFAP expression can be monitored by measuring GFAP levels in blood of 5×FAD mice.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

1: A method of decreasing glial fibrillary acidic protein (GFAP) levels in a subject in need thereof, the method comprising:
administering to the subject a therapeutically effective amount of a 15-PGDH inhibitor.

2: The method of claim 1, wherein the subject has or is at risk of cognitive decline, neural injury, neurodegeneration, and/or a neurodegenerative and/or neuropsychiatric condition, disease, and/or disorder that is associated with aberrant and/or elevated GFAP levels.

3: The method of claim 1, wherein the therapeutically effective amount is an amount effective to decrease blood GFAP levels by at least about 5% or more.

4: The method of claim 3, wherein the cognitive decline, neural injury, neurodegeneration, and/or a neurodegenerative and/or neuropsychiatric condition, disease, and/or disorder is further caused by and/or associated with elevated or aberrant 15-PGDH activity in the central or peripheral nervous system of the subject and the amount of 15-PGDH inhibitor administered to the subject is an amount effective to inhibit 15-PGDH activity in the central or peripheral nervous system.

5: The method of claim 3, wherein the neural injury, neurodegeneration, and/or neurodegenerative and/or neuropsychiatric condition, disease, and/or disorder comprises at least one of subarachnoid hemorrhage, schizophrenia, depression, bipolar disorder, normal aging, epilepsy, traumatic brain injury and/or a visual symptom associated therewith, post-traumatic stress disorder, Parkinson's disease, Parkinson Plus syndromes, Lewy Body Dementia, multiple system atrophy, corticobasal neurodegeneration, progressive supranuclear palsy, Alexander's disease, Alzheimer's disease, Alzheimer's disease related dementias, Down syndrome, spinocerebellar ataxia, amyotrophic lateral sclerosis, Huntington's disease, stroke, brain radiation therapy, chronic stress, abuse or cellular toxicity of a neuro-active drug, retinal degeneration, spinal cord injury, peripheral nerve injury, idiopathic peripheral neuropathy, cognitive decline and/or general frailty associated with normal aging and/or chemotherapy, chemotherapy induced neuropathy, concussive injury, peripheral nerve crush injury, peripheral neuropathy, diabetic neuropathy, post-traumatic headache, multiple sclerosis, retinal degeneration and dystrophy, Leber congenital amaurosis, retinitis pigmentosa, cone-rod dystrophy, microphthalmia, anophthalmia, myopia, and hyperopia, spinal cord injury, traumatic spinal cord injury, peripheral nerve injury, retinal neuronal death related diseases, retinal trauma, Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy, Hereditary spastic paraparesis, Friedreich ataxia, Non-Friedreich ataxia, Spinocerebellar atrophies, Amyloidoses, Metabolic-related neurodegenerative disorders, Toxin-related neurodegenerative disorders, Multiple sclerosis, Charcot Marie Tooth, Diabetic neuropathy, Metabolic neuropathies, Endocrine neuropathies, Creutzfeldt-Jacob Disease, Primary progressive aphasia, Frontotemporal Lobar Degeneration, Cortical blindness, Shy-Drager Syndrome, Diffuse cerebral cortical atrophy of non-Alzheimer type, Lewy-body dementia, Pick disease, Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, Tourette syndrome, or viral infection.

6: The method of claim 1, wherein the subject has astrogliosis or an astrocytopathy.

7. (canceled)

8: The method of claim 1, wherein the subject has memory loss and/or cognitive decline and the 15-PGDH inhibitor is administered at amount effective to improve memory and/or cognition.

9: The method of claim 1, wherein the 15-PGDH inhibitor can inhibit enzymatic activity of recombinant 15-PGDH at an IC₅₀ of less than 1 μM at a recombinant 15-PGDH concentration of about 5 nM to about 10 nM.

10: The method of claim 1, wherein the 15-PGDH inhibitor has the following formula (V): 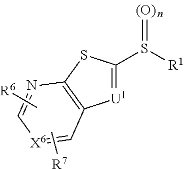
or a pharmaceutically acceptable salt, tautomer, or solvate thereof;
wherein n is 0-2
X⁶ is independently is N or CR^{C}
R¹, R⁶, R⁷, and R^{c} are the same or different each independently hydrogen or a substituted or unsubstituted group selected from C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms, C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₅-C₂₀ aryl amino, C₂-C₂₄ alkylamido, C₂-C₂₄ alkylamido substituted with a hydroxyl, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkylethers, phosphates, and phosphate esters, groups incorporating amino acids or other moieties expected to bear positive or negative charge at physiological pH, and combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl; and
U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X═H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X═H, F, Cl, Br, or I), CN, (C═O)—R′, (C═O)N(R′)₂, COOR′ (wherein R′ is H or a lower alkyl group), and R³ or R⁴ may be absent.

11: The method of claim 2, further comprising measuring GFAP levels in blood of the subject following administration of the 15-PGDH inhibitor and comparing the measured GFAP levels to a control, wherein a decrease in blood GFAP levels is indicative of efficacy of the 15-PGDH inhibitor in treating the cognitive decline, neural injury, neurodegeneration, and/or the neurodegenerative and/or neuropsychiatric condition, disease, and/or disorder in the subject.

12: The method of claim 11, wherein the control is a level of GFAP in blood of a subject prior to administration of the 15-PGDH inhibitor.

13: A method of treating cognitive decline, neural injury, neurodegeneration, and/or a neurodegenerative and/or neuropsychiatric condition, disease, and/or disorder associated with aberrant and/or elevated GFAP levels in a subject in need thereof, the method comprising:
measuring GFAP levels in blood of a subject; and
administering to the subject an amount of a 15-PGDH inhibitor effective to decrease GFAP levels in blood of the subject.

14: The method of claim 13, wherein the amount of 15-PGDH inhibitor is effective to decrease blood GFAP levels by at least about 5% or more.

15: The method of claim 13, wherein the cognitive decline, neural injury, neurodegeneration, and/or a neurodegenerative and/or neuropsychiatric condition, disease, and/or disorder is further caused by and/or associated with elevated or aberrant 15-PGDH activity in the central or peripheral nervous system of the subject and the amount of 15-PGDH inhibitor administered to the subject is an amount effective to inhibit 15-PGDH activity in the central or peripheral nervous system.

16: The method of claim 13, wherein the neural injury, neurodegeneration, and/or neurodegenerative and/or neuropsychiatric condition, disease, and/or disorder comprises at least one of subarachnoid hemorrhage, schizophrenia, depression, bipolar disorder, normal aging, epilepsy, traumatic brain injury and/or a visual symptom associated therewith, post-traumatic stress disorder, Parkinson's disease, Parkinson Plus syndromes, Lewy Body Dementia, multiple system atrophy, corticobasal neurodegeneration, progressive supranuclear palsy, Alexander's disease, Alzheimer's disease, Alzheimer's disease related dementias, Down syndrome, spinocerebellar ataxia, amyotrophic lateral sclerosis, Huntington's disease, stroke, brain radiation therapy, chronic stress, abuse or cellular toxicity of a neuro-active drug, retinal degeneration, spinal cord injury, peripheral nerve injury, idiopathic peripheral neuropathy, cognitive decline and/or general frailty associated with normal aging and/or chemotherapy, chemotherapy induced neuropathy, concussive injury, peripheral nerve crush injury, peripheral neuropathy, diabetic neuropathy, post-traumatic headache, multiple sclerosis, retinal degeneration and dystrophy, Leber congenital amaurosis, retinitis pigmentosa, cone-rod dystrophy, microphthalmia, anophthalmia, myopia, and hyperopia, spinal cord injury, traumatic spinal cord injury, peripheral nerve injury, retinal neuronal death related diseases, retinal trauma, Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy, Hereditary spastic paraparesis, Friedreich ataxia, Non-Friedreich ataxia, Spinocerebellar atrophies, Amyloidoses, Metabolic-related neurodegenerative disorders, Toxin-related neurodegenerative disorders, Multiple sclerosis, Charcot Marie Tooth, Diabetic neuropathy, Metabolic neuropathies, Endocrine neuropathies, Creutzfeldt-Jacob Disease, Primary progressive aphasia, Frontotemporal Lobar Degeneration, Cortical blindness, Shy-Drager Syndrome, Diffuse cerebral cortical atrophy of non-Alzheimer type, Lewy-body dementia, Pick disease, Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, Tourette syndrome, or viral infection.

17: The method of claim 13, wherein the subject has astrogliosis or an astrocytopathy.

18. (canceled)

19: The method of claim 13, wherein the subject has memory loss and/or cognitive decline and the 15-PGDH inhibitor is administered at amount effective to improve memory and/or cognition.

20: The method of claim 13, wherein the 15-PGDH inhibitor can inhibit enzymatic activity of recombinant 15-PGDH at an IC₅₀ of less than 1 μM at a recombinant 15-PGDH concentration of about 5 nM to about 10 nM.

21: The method of claim 13, wherein the 15-PGDH inhibitor has the following formula (V): 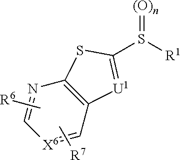
or a pharmaceutically acceptable salt, tautomer, or solvate thereof;
wherein n is 0-2
X⁶ is independently is N or CR^{C}
R¹, R⁶, R⁷, and R^{C} are the same or different each independently hydrogen or a substituted or unsubstituted group selected from C₁-C₂₄ alkyl, C₂-C₂₄ alkenyl, 02-C₂₄ alkynyl, C₃-C₂₀ aryl, heteroaryl, heterocycloalkenyl containing from 5-6 ring atoms, C₆-C₂₄ alkaryl, C₆-C₂₄ aralkyl, halo, —Si(C₁-C₃ alkyl)₃, hydroxyl, sulfhydryl, 01-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₅-C₂₀ aryl amino, C₂-C₂₄ alkylamido, C₂-C₂₄ alkylamido substituted with a hydroxyl, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkylethers, phosphates, and phosphate esters, groups incorporating amino acids or other moieties expected to bear positive or negative charge at physiological pH, and combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl; and
U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X═H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X═H, F, Cl, Br, or I), CN, (C═O)—R′, (C═O)N(R′)₂, COOR′ (wherein R′ is H or a lower alkyl group), and R³ or R⁴ may be absent.

22: The method of claim 13, further comprising measuring GFAP levels in blood of subject following administration of the 15-PGDH inhibitor and comparing the measured GFAP levels to a control, wherein a decrease in blood GFAP levels is indicative of efficacy of the 15-PGDH inhibitor in treating the cognitive decline, neural injury, neurodegeneration, and/or the neurodegenerative and/or neuropsychiatric condition, disease, and/or disorder in the subject.

23: The method of claim 22, wherein the control is a level of GFAP in blood of a subject prior to administration of the 15-PGDH inhibitor.
